(19) United States
(12) Patent Application Publication
(10) Pub. No.: US 2025/0115582 A1
(43) Pub. Date: Apr. 10, 2025

(54) MOLECULAR GLUE DEGRADERS AND METHODS OF USING THE SAME

(71) Applicant: Monte Rosa Therapeutics, Inc., Boston, MA (US)

(72) Inventors: Laura Ann McAllister, Basel (CH); Lars Wiedmer, Basel (CH); Ètienne Donckele, Basel (CH); Bernhard Fasching, Basel (CH); Nina Ilic-Widlund, Basel (CH)

(21) Appl. No.: 18/982,872

(22) Filed: Dec. 16, 2024

Related U.S. Application Data
(63) Continuation of application No. PCT/US2023/025593, filed on Jun. 16, 2023.
(60) Provisional application No. 63/352,893, filed on Jun. 16, 2022.

(51) Int. Cl. C07D 401/14; A61K 31/451; A61K 31/454; A61K 31/4545; A61K 31/497; A61K 31/506; A61K 45/06; C07D 211/42; C07D 401/04; C07D 401/12; C07D 413/14; C07D 417/14

(52) U.S. Cl. CPC C07D 401/14; A61K 31/451; A61K 31/454; A61K 31/4545; A61K 31/497; A61K 31/506; A61K 45/06; C07D 211/42; C07D 401/04; C07D 401/12; C07D 413/14; C07D 417/14

(57) ABSTRACT

Described herein, in part, are compounds that mediate the degradation of casein kinase 1α (CK1α), and are therefore useful in the treatment of various disorders, such as cancer.

### RELATED APPLICATIONS

This application is a continuation of International Patent Application No. PCT/US2023/025593, filed Jun. 16, 2023, which claims priority to U.S. Provisional Patent Application No. 63/352,893, filed on Jun. 16, 2022, the contents of which are incorporated by reference in their entireties.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as modulators of cereblon to mediate the degradation of a protein, and are therefore are useful in the treatment of disorders, such as cancer. For example, it has been found that compounds of the present disclosure mediate the targeted degradation of the protein casein kinase 1α (CK1α).

In one aspect, described herein is a compound of Formula (I):

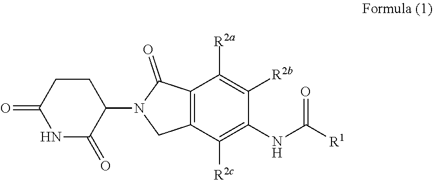

or a pharmaceutically acceptable salt thereof, wherein: R¹ is selected from the group consisting of phenyl, heteroaryl, cycloalkyl, and heterocyclyl, wherein each of phenyl, heteroaryl, cycloalkyl, and heterocyclyl is optionally substituted with one or more occurrences of R^{A}; each of R^{2a}, R^{2b}, and R^{2c} is independently selected from the group consisting of H, C₁₋₃alkyl, and C₁₋₃alkoxy; each occurrence of R^{A} is independently selected from the group consisting of halogen, C₁₋₆alkyl, cyano, cycloalkyl heteroaryl, and phenyl, wherein each of C₁₋₆alkyl, cycloalkyl, heteroaryl, and phenyl is optionally substituted with one or more occurrences of R^{B}, or one or more groups of two occurrences of R^{A}, together with the carbon to which they are attached, form oxo; and each occurrence of R^{B} is independently selected from the group consisting of halogen and C₁₋₆alkyl.

In an aspect, also provided herein is a pharmaceutical composition comprising a compound described herein, or pharmaceutically acceptable salt thereof, and a pharmaceutically acceptable excipient.

In another aspect, provided herein is a method of degrading CK1a in a subject suffering from cancer, comprising administering to the subject an effective amount of a compound described herein, or pharmaceutically acceptable salt thereof, or a pharmaceutical composition described herein.

In another aspect, provided herein is a method of treating cancer in a subject in need thereof, comprising administering to the subject a therapeutically effective amount of a compound of described herein, or pharmaceutically acceptable salt thereof, or a pharmaceutical composition described herein.

In another aspect, provided herein is a method of treating a solid tumor in a subject in need thereof, comprising administering to the subject a therapeutically effective amount of a compound of described herein, or pharmaceutically acceptable salt thereof, or a pharmaceutical composition described herein.

In another aspect, provided herein is a method of treating a liquid tumor in a subject in need thereof, comprising administering to the subject a therapeutically effective amount of a compound of described herein, or pharmaceutically acceptable salt thereof, or a pharmaceutical composition described herein.

### BRIEF DESCRIPTION OF THE DRAWINGS

FIG. 1 depicts effects of Compound 147 on CK1a levels in an HEK293 cell line in comparison to effects of Compound 147 on CK1a levels in a cereblon (CRBN) knock-out HEK293 cell line. The data show that Compound 147 decreases CK1a levels in the HEK293 cell line while in the presence of cereblon, whereas Compound 147 demonstrates no effect on CK1a levels in the absence of cereblon in the cereblon knock-out HEK293 cell line.

FIG. 2 depicts time-dependent plasma concentrations for Mouse oral PK at 10 mg/kg for compound 154 and compound 147.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (I):

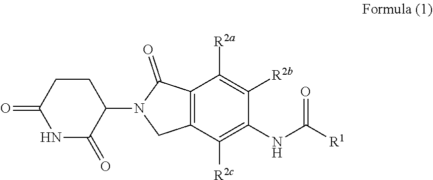

or a pharmaceutically acceptable salt thereof, wherein: R¹ is selected from the group consisting of phenyl, heteroaryl, cycloalkyl, and heterocyclyl, wherein each of phenyl, heteroaryl, cycloalkyl, and heterocyclyl is optionally substituted with one or more occurrences of R^{A}; each of R^{2a}, R^{2b}, and R^{2c} is independently selected from the group consisting of H, C₁₋₃alkyl, and C₁₋₃alkoxy; each occurrence of R^{A} is independently selected from the group consisting of halogen, C₁₋₆alkyl, cyano, cycloalkyl heteroaryl, and phenyl, wherein each of C₁₋₆alkyl, cycloalkyl, heteroaryl, and phenyl is optionally substituted with one or more occurrences of R^{B}, or one or more groups of two occurrences of R^{A}, together with the carbon to which they are attached, form oxo; and each occurrence of R^{B} is independently selected from the group consisting of halogen and C₁₋₆alkyl.

In one aspect, described herein is a compound of Formula (I):

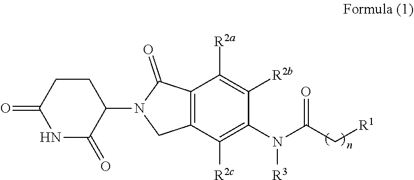

or a pharmaceutically acceptable salt thereof, wherein: R¹ is selected from the group consisting of phenyl, heteroaryl, cycloalkyl, and heterocyclyl, wherein each of phenyl, heteroaryl, cycloalkyl, and heterocyclyl is optionally substituted with one or more occurrences of R^{A}; each of R^{2a}, R^{2b}, and R^{2c} is independently selected from the group consisting of H, C₁₋₃alkyl, and C₁₋₃alkoxy; each occurrence of R^{A} is independently selected from the group consisting of oxo, halogen, C₁₋₆alkyl, C₁₋₃alkoxy, cyano, cycloalkyl, heteroaryl, and phenyl, wherein each of C₁₋₆alkyl, cycloalkyl, heteroaryl, and phenyl is optionally substituted with one or more occurrences of R^{B}, or one or more groups of two occurrences of R^{A}, together with the carbon to which they are attached, form oxo; each occurrence of R^{B} is independently selected from the group consisting of halogen, C₁₋₆alkyl, and C₁₋₆alkoxy; R³ is hydrogen, C₁₋₃alkyl, or C₁₋₃haloalkyl, or R¹ and R³ can, together with the atoms to which they are attached, form a 9-10 membered heterocyclyl optionally substituted by one or more oxo; and n is 0 or 1.

In some embodiments, R¹ is substituted or unsubstituted 5 or 6-membered heteroaryl. In some embodiments, R¹ is 5 or 6-membered heteroaryl optionally substituted with one or more occurrences of R^{A}. In some embodiments, R¹ is phenyl optionally substituted with one or more occurrences of R^{A}. In some embodiments, R¹ is pyridinyl optionally substituted with one or more occurrences of R^{A}. In some embodiments, R¹ is thiazolyl, imidazolyl or oxazolyl optionally substituted with one or more occurrences of R^{A}. In some embodiments, R¹ is thiazolyl or oxazolyl optionally substituted with one or more occurrences of R^{A}. In some embodiments, R¹ is 5 or 6-membered heteroaryl substituted with one or more occurrences of R^{A}. In some embodiments, R¹ is phenyl substituted with one or more occurrences of R^{A}. In some embodiments, R¹ is 5 or 6-membered heteroaryl substituted with one or two occurrences of R^{A}. In some embodiments, R¹ is pyridinyl substituted with one or two occurrences of R^{A}. In some embodiments, R¹ is phenyl substituted with one or two occurrences of R^{A}. In some embodiments, R¹ is 5 or 6-membered heteroaryl substituted with one or two occurrences of halogen. In some embodiments, R¹ is pyridinyl substituted with one or two occurrences of halogen. In some embodiments, R¹ is phenyl substituted with one or two occurrences of halogen. In some embodiments, R¹ is 5 or 6-membered heteroaryl substituted with methyl, wherein methyl is optionally substituted with one more occurrences of halogen. In some embodiments, R¹ is pyridinyl substituted with methyl, wherein methyl is optionally substituted with one more occurrences of halogen.

In some embodiments, R¹ is substituted or unsubstituted aryl. In some embodiments, R¹ is phenyl substituted with methyl, wherein methyl is optionally substituted with one more occurrences of halogen. In some embodiments, R¹ is 8-10 membered bicyclic heterocyclyl optionally substituted by one or more oxo. In some embodiments, R¹ is indolinyl optionally substituted by one or more oxo. In some embodiments, R¹ is 9-10 membered bicyclic cycloalkyl. In some embodiments, 5-6 membered monocyclic heterocyclyl optionally substituted by one or more C₁₋₆alkyl, wherein the alkyl is optionally substituted by one or more halogen.

In some embodiments, R^{2a}, R^{2b}, and R^{2c} are H. In some embodiments, each of R^{2a}, R^{2b}, and R^{2c} is independently selected from the group consisting of C₁₋₃alkyl and C₁₋₃ alkoxy. In some embodiments, each of R^{2a}, R^{2b}, and R^{2c} is independently selected from the group consisting of methyl, ethyl, and methoxy. In some embodiments, each occurrence of R^{A} is independently selected from the group consisting of halogen, C₁₋₃alkyl, and cyano, wherein C₁₋₃alkyl is optionally substituted with one or more occurrences of halogen. In some embodiments, each occurrence of R^{A} is independently selected from the group consisting of F, Cl, methyl, ethyl, isopropyl, and cyano, wherein each of methyl, ethyl, and isopropyl is optionally substituted with one or more occurrences of F. In some embodiments, each occurrence of R^{A} is independently selected from the group consisting of Cl and methyl. In some embodiments, each occurrence of R^{A} is independently selected from the group consisting of phenyl and heteroaryl. In some embodiments, each occurrence of R^{B} is independently selected from the group consisting of F, Cl, methyl, ethyl, and cyano, wherein each of methyl and ethyl is optionally substituted with one or more occurrences of F.

In some embodiments, R¹ is selected from the group consisting of:

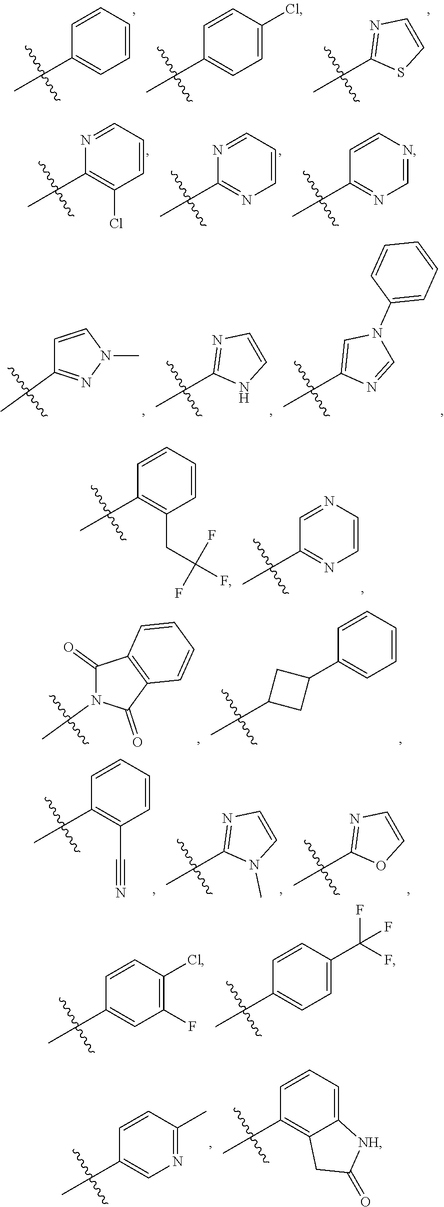

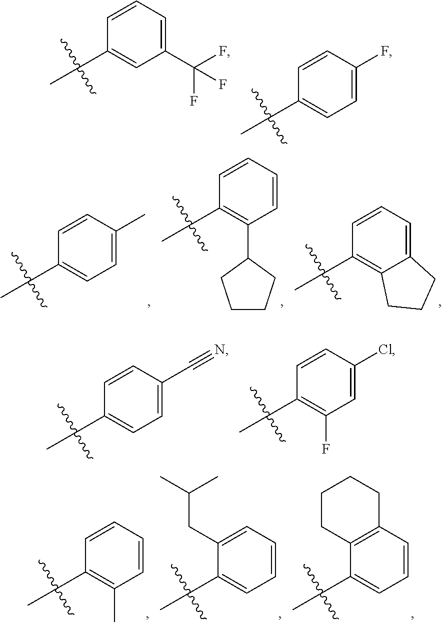

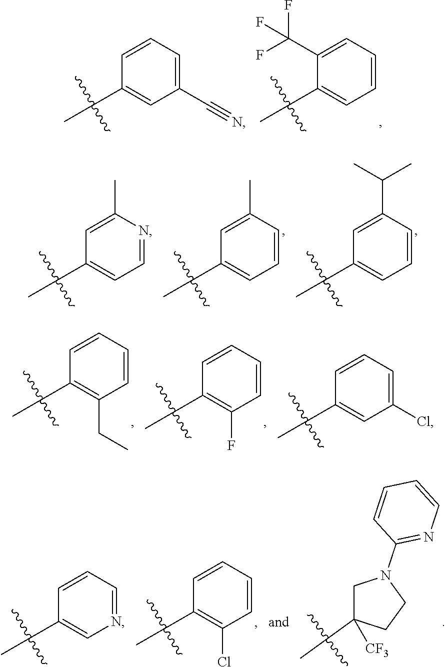

In some embodiments, R¹ is selected from the group consisting of:

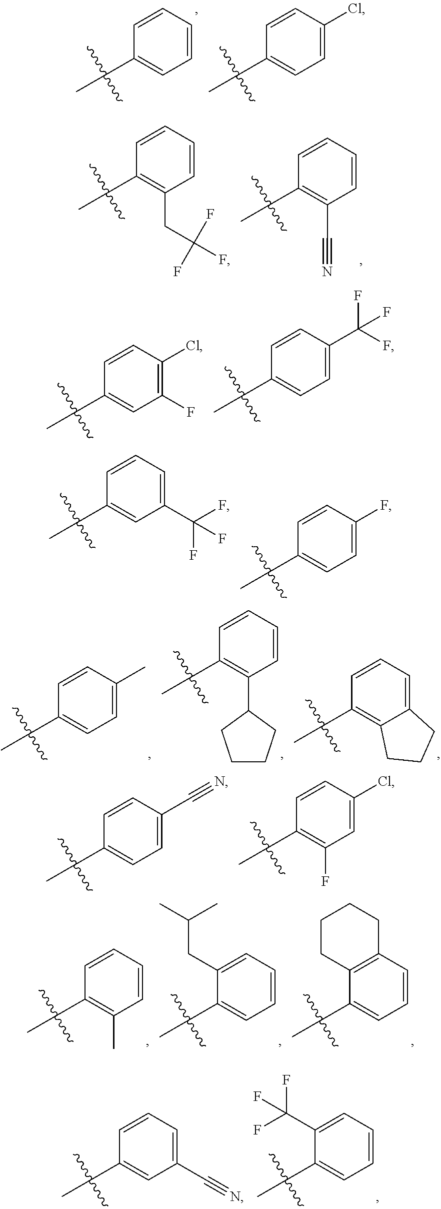

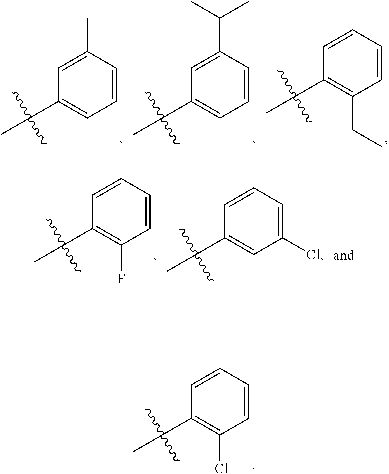

In some embodiments, R¹ is selected from the group consisting of:

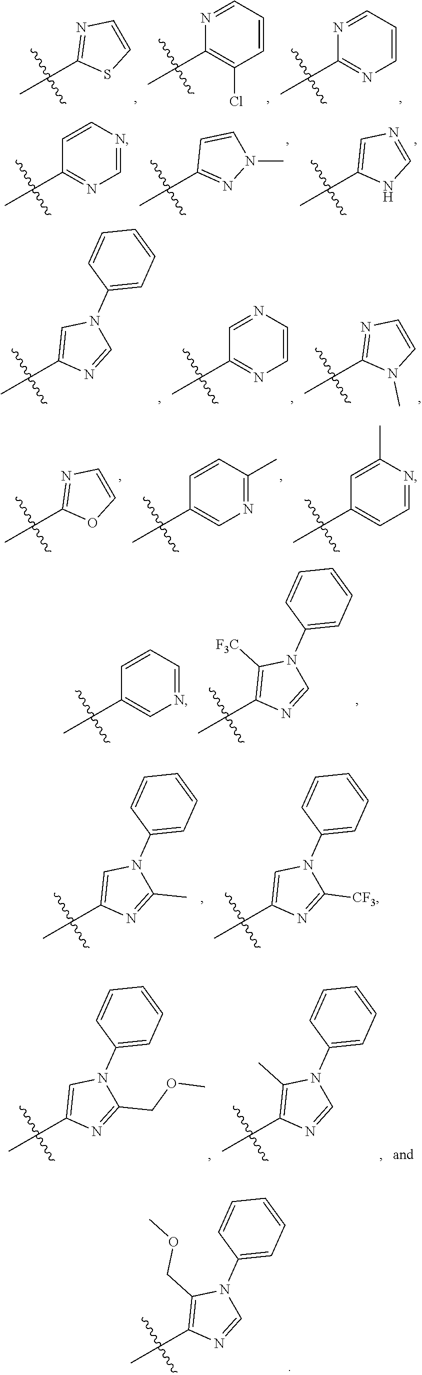

In some embodiments, n is 1. In some embodiments, n is 0.

Provided herein, in an aspect, is a compound described in Table 1 below. Table 1 also includes the compound number of each compound in accordance with the contents of the present specification.

In an embodiment, a compound of the disclosure is a compound identified in Table 1 below, or a pharmaceutically acceptable salt thereof.

TABLE 1
Exemplary compounds.

| Compound | |
|---|---|
| Number | Structure |
| 101 | 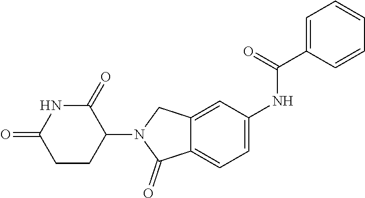 [SMILES: O=C1CCC(N2Cc3cc(NC(=O)c4ccccc4)ccc3C2=O)C(=O)N1] |
| 102 | 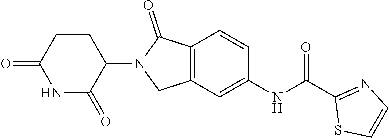 [SMILES: O=C1CCC(N2Cc3cc(NC(=O)c4nccs4)ccc3C2=O)C(=O)N1] |
| 103 | 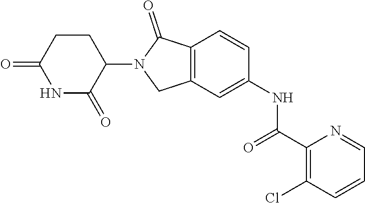 [SMILES: O=C1CCC(N2Cc3cc(NC(=O)c4ncccc4Cl)ccc3C2=O)C(=O)N1] |
| 104 | 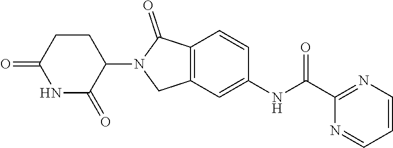 [SMILES: O=C1CCC(N2Cc3cc(NC(=O)c4ncccn4)ccc3C2=O)C(=O)N1] |
| 105 | 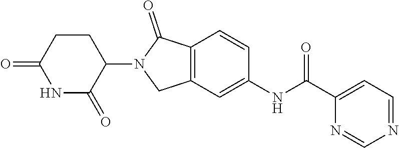 [SMILES: O=C1CCC(N2Cc3cc(NC(=O)c4ccncn4)ccc3C2=O)C(=O)N1] |
| 106 | 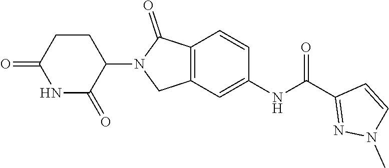 [SMILES: Cn1ccc(C(=O)Nc2ccc3c(c2)CN(C2CCC(=O)NC2=O)C3=O)n1] |
| 107 | 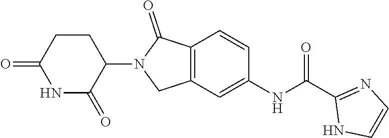 [SMILES: O=C1CCC(N2Cc3cc(NC(=O)c4ncc[nH]4)ccc3C2=O)C(=O)N1] |
| 108 | 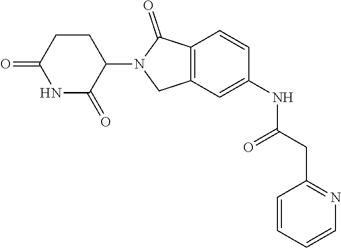 [SMILES: O=C1CCC(N2Cc3cc(NC(=O)Cc4ccccn4)ccc3C2=O)C(=O)N1] |
| 109 | 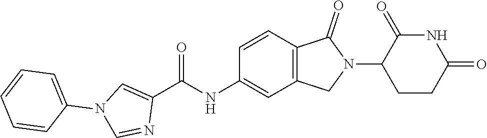 [SMILES: O=C1CCC(N2Cc3cc(NC(=O)c4cn(-c5ccccc5)cn4)ccc3C2=O)C(=O)N1] |
| 110 | 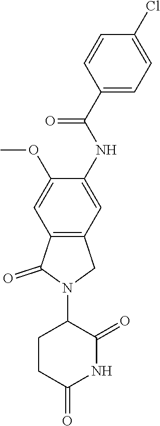 [SMILES: COc1cc2c(cc1NC(=O)c1ccc(Cl)cc1)CN(C1CCC(=O)NC1=O)C2=O] |
| 111 | 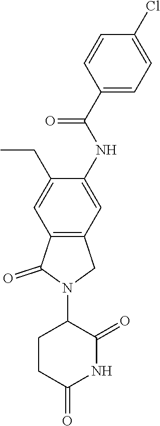 [SMILES: CCc1cc2c(cc1NC(=O)c1ccc(Cl)cc1)CN(C1CCC(=O)NC1=O)C2=O] |
| 112 | 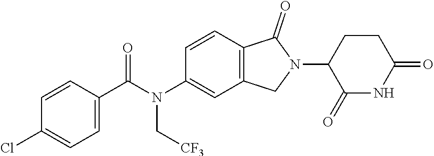 [SMILES: O=C1CCC(N2Cc3cc(N(CC(F)(F)F)C(=O)c4ccc(Cl)cc4)ccc3C2=O)C(=O)N1] |
| 113 | 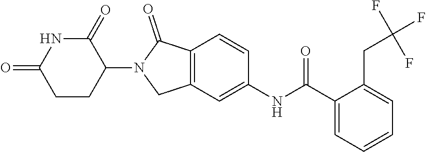 [SMILES: O=C1CCC(N2Cc3cc(NC(=O)c4ccccc4CC(F)(F)F)ccc3C2=O)C(=O)N1] |
| 114 | 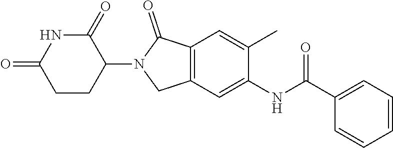 [SMILES: Cc1cc2c(cc1NC(=O)c1ccccc1)CN(C1CCC(=O)NC1=O)C2=O] |
| 115 | 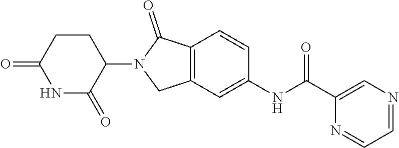 [SMILES: O=C1CCC(N2Cc3cc(NC(=O)c4cnccn4)ccc3C2=O)C(=O)N1] |
| 116 | 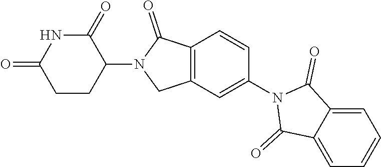 [SMILES: O=C1CCC(N2Cc3cc(N4C(=O)c5ccccc5C4=O)ccc3C2=O)C(=O)N1] |
| 117 | 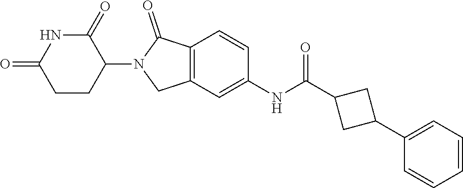 [SMILES: O=C1CCC(N2Cc3cc(NC(=O)C4CC(c5ccccc5)C4)ccc3C2=O)C(=O)N1] |
| 118 | 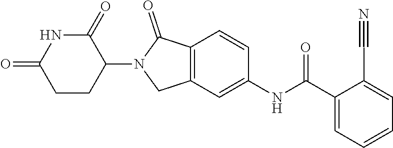 [SMILES: N#Cc1ccccc1C(=O)Nc1ccc2c(c1)CN(C1CCC(=O)NC1=O)C2=O] |
| 119 | 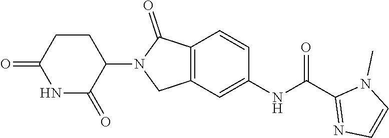 [SMILES: Cn1ccnc1C(=O)Nc1ccc2c(c1)CN(C1CCC(=O)NC1=O)C2=O] |
| 120 | 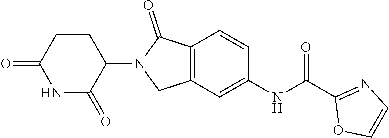 [SMILES: O=C1CCC(N2Cc3cc(NC(=O)c4ncco4)ccc3C2=O)C(=O)N1] |
| 121 | 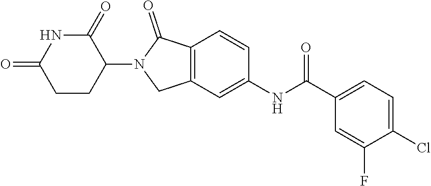 [SMILES: O=C1CCC(N2Cc3cc(NC(=O)c4ccc(Cl)c(F)c4)ccc3C2=O)C(=O)N1] |
| 122 | 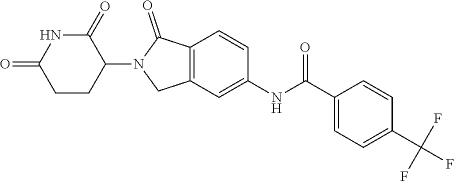 [SMILES: O=C1CCC(N2Cc3cc(NC(=O)c4ccc(C(F)(F)F)cc4)ccc3C2=O)C(=O)N1] |
| 123 | 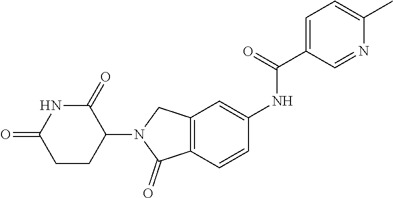 [SMILES: Cc1ccc(C(=O)Nc2ccc3c(c2)CN(C2CCC(=O)NC2=O)C3=O)cn1] |
| 124 | 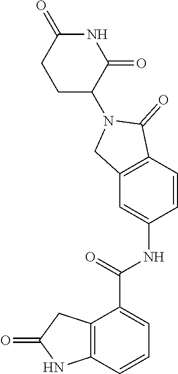 [SMILES: O=C1CCC(N2Cc3cc(NC(=O)c4cccc5c4CC(=O)N5)ccc3C2=O)C(=O)N1] |
| 125 | 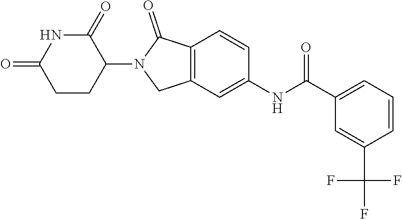 [SMILES: O=C1CCC(N2Cc3cc(NC(=O)c4cccc(C(F)(F)F)c4)ccc3C2=O)C(=O)N1] |
| 126 | 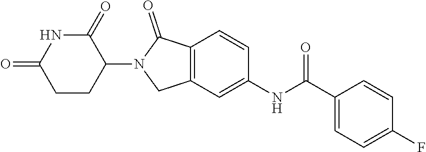 [SMILES: O=C1CCC(N2Cc3cc(NC(=O)c4ccc(F)cc4)ccc3C2=O)C(=O)N1] |
| 127 | 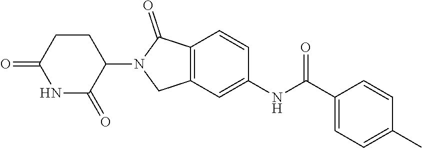 [SMILES: Cc1ccc(C(=O)Nc2ccc3c(c2)CN(C2CCC(=O)NC2=O)C3=O)cc1] |
| 128 | 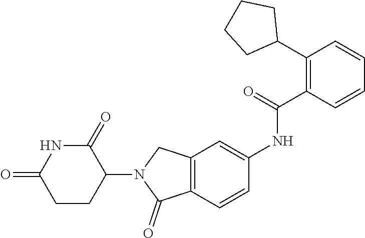 [SMILES: O=C1CCC(N2Cc3cc(NC(=O)c4ccccc4C4CCCC4)ccc3C2=O)C(=O)N1] |
| 129 | 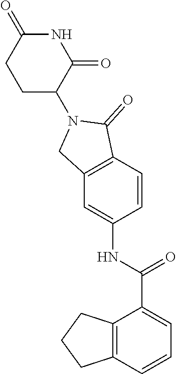 [SMILES: O=C1CCC(N2Cc3cc(NC(=O)c4cccc5c4CCC5)ccc3C2=O)C(=O)N1] |
| 130 | 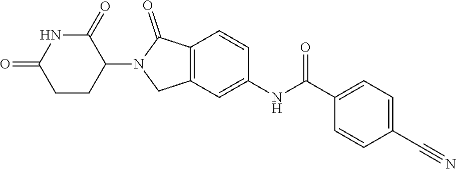 [SMILES: N#Cc1ccc(C(=O)Nc2ccc3c(c2)CN(C2CCC(=O)NC2=O)C3=O)cc1] |
| 131 | 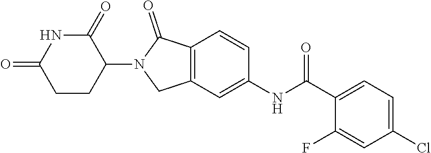 [SMILES: O=C1CCC(N2Cc3cc(NC(=O)c4ccc(Cl)cc4F)ccc3C2=O)C(=O)N1] |
| 132 | 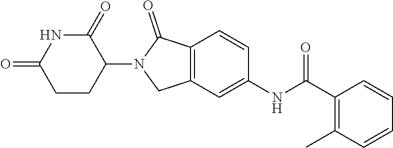 [SMILES: Cc1ccccc1C(=O)Nc1ccc2c(c1)CN(C1CCC(=O)NC1=O)C2=O] |
| 133 | 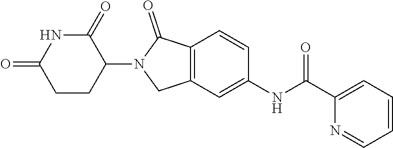 [SMILES: O=C1CCC(N2Cc3cc(NC(=O)c4ccccn4)ccc3C2=O)C(=O)N1] |
| 134 | 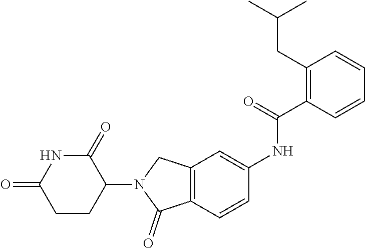 [SMILES: CC(C)Cc1ccccc1C(=O)Nc1ccc2c(c1)CN(C1CCC(=O)NC1=O)C2=O] |
| 135 | 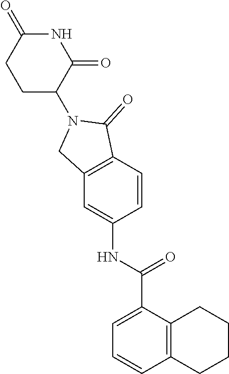 [SMILES: O=C1CCC(N2Cc3cc(NC(=O)c4cccc5c4CCCC5)ccc3C2=O)C(=O)N1] |
| 136 | 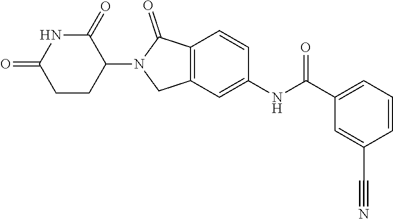 [SMILES: N#Cc1cccc(C(=O)Nc2ccc3c(c2)CN(C2CCC(=O)NC2=O)C3=O)c1] |
| 137 | 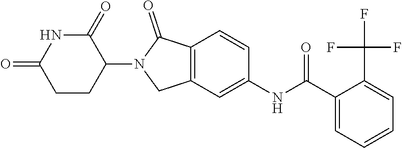 [SMILES: O=C1CCC(N2Cc3cc(NC(=O)c4ccccc4C(F)(F)F)ccc3C2=O)C(=O)N1] |
| 138 | 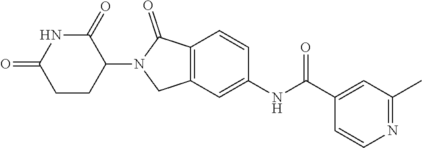 [SMILES: Cc1cc(C(=O)Nc2ccc3c(c2)CN(C2CCC(=O)NC2=O)C3=O)ccn1] |
| 139 | 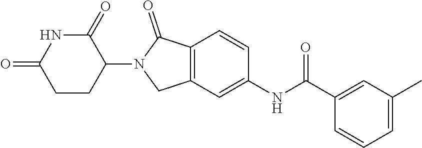 [SMILES: Cc1cccc(C(=O)Nc2ccc3c(c2)CN(C2CCC(=O)NC2=O)C3=O)c1] |
| 140 | 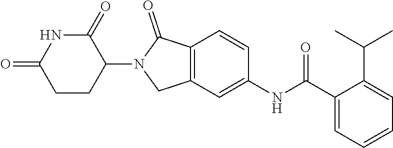 [SMILES: CC(C)c1ccccc1C(=O)Nc1ccc2c(c1)CN(C1CCC(=O)NC1=O)C2=O] |
| 141 | 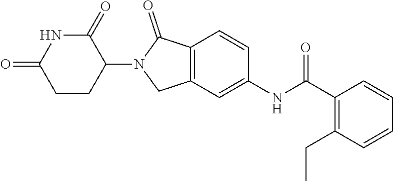 [SMILES: CCc1ccccc1C(=O)Nc1ccc2c(c1)CN(C1CCC(=O)NC1=O)C2=O] |
| 142 | 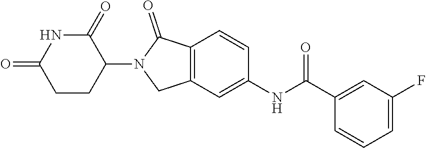 [SMILES: O=C1CCC(N2Cc3cc(NC(=O)c4cccc(F)c4)ccc3C2=O)C(=O)N1] |
| 143 | 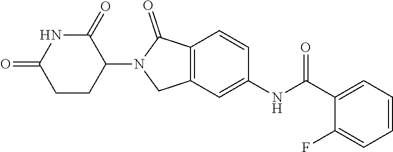 [SMILES: O=C1CCC(N2Cc3cc(NC(=O)c4ccccc4F)ccc3C2=O)C(=O)N1] |
| 144 | 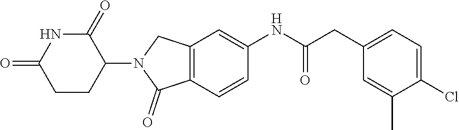 [SMILES: Cc1cc(CC(=O)Nc2ccc3c(c2)CN(C2CCC(=O)NC2=O)C3=O)ccc1Cl] |
| 145 | 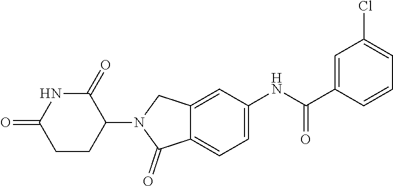 [SMILES: O=C1CCC(N2Cc3cc(NC(=O)c4cccc(Cl)c4)ccc3C2=O)C(=O)N1] |
| 146 | 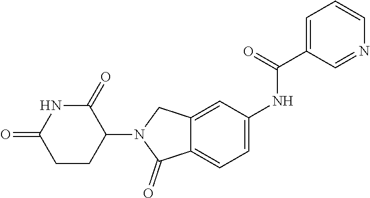 [SMILES: O=C1CCC(N2Cc3cc(NC(=O)c4cccnc4)ccc3C2=O)C(=O)N1] |
| 147 | 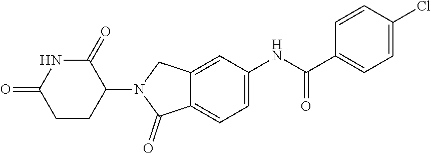 [SMILES: O=C1CCC(N2Cc3cc(NC(=O)c4ccc(Cl)cc4)ccc3C2=O)C(=O)N1] |
| 148 | 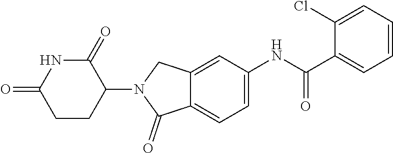 [SMILES: O=C1CCC(N2Cc3cc(NC(=O)c4ccccc4Cl)ccc3C2=O)C(=O)N1] |
| 149 | 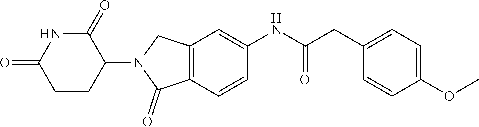 [SMILES: COc1ccc(CC(=O)Nc2ccc3c(c2)CN(C2CCC(=O)NC2=O)C3=O)cc1] |
| 150 | 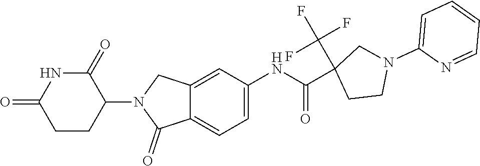 [SMILES: O=C1CCC(N2Cc3cc(NC(=O)C4(C(F)(F)F)CCN(c5ccccn5)C4)ccc3C2=O)C(=O)N1] |
| 151 | 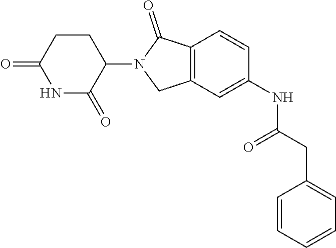 [SMILES: O=C1CCC(N2Cc3cc(NC(=O)Cc4ccccc4)ccc3C2=O)C(=O)N1] |
| 152 | 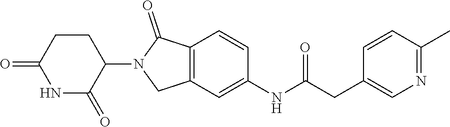 [SMILES: Cc1ccc(CC(=O)Nc2ccc3c(c2)CN(C2CCC(=O)NC2=O)C3=O)cn1] |
| 153 | 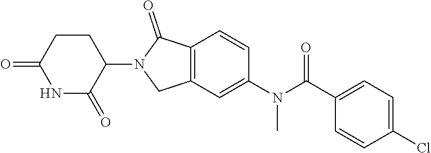 [SMILES: CN(C(=O)c1ccc(Cl)cc1)c1ccc2c(c1)CN(C1CCC(=O)NC1=O)C2=O] |
| 154 | 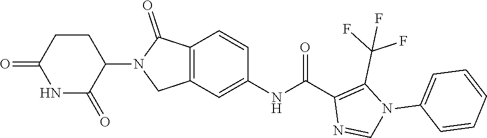 [SMILES: O=C1CCC(N2Cc3cc(NC(=O)c4ncn(-c5ccccc5)c4C(F)(F)F)ccc3C2=O)C(=O)N1] |
| 155 | 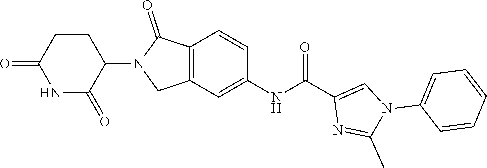 [SMILES: Cc1nc(C(=O)Nc2ccc3c(c2)CN(C2CCC(=O)NC2=O)C3=O)cn1-c1ccccc1] |
| 156 | 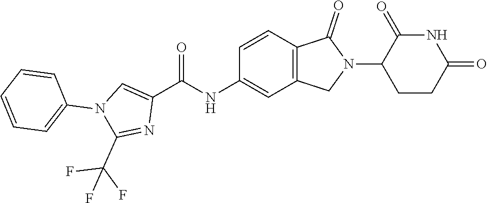 [SMILES: O=C1CCC(N2Cc3cc(NC(=O)c4cn(-c5ccccc5)c(C(F)(F)F)n4)ccc3C2=O)C(=O)N1] |
| 157 | 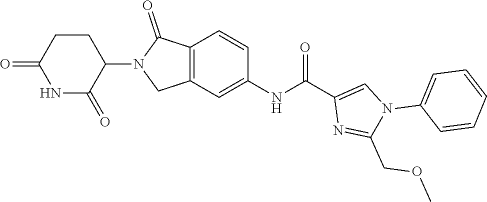 [SMILES: COCc1nc(C(=O)Nc2ccc3c(c2)CN(C2CCC(=O)NC2=O)C3=O)cn1-c1ccccc1] |
| 158 | 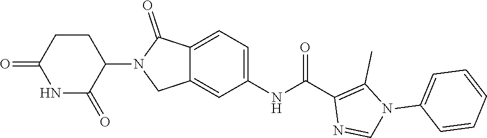 [SMILES: Cc1c(C(=O)Nc2ccc3c(c2)CN(C2CCC(=O)NC2=O)C3=O)ncn1-c1ccccc1] |
| 159 | 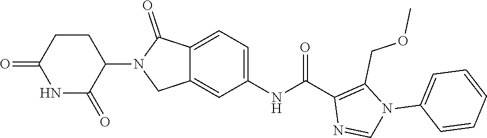 [SMILES: COCc1c(C(=O)Nc2ccc3c(c2)CN(C2CCC(=O)NC2=O)C3=O)ncn1-c1ccccc1] |

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to degrade casein kinase 1α (CK1α) for the treatment of prevention of a disorder.

Casein kinase I (CK1) is a monomeric serine-threonine protein kinase with 7 isoforms: alpha, beta, gamma1, gamma2, gamma3, delta and epsilon. CK1 is involved in many cellular processes including DNA repair, cell division, nuclear localization and membrane transport. Isoforms are also integral to development. CK1a (casein kinase 1 alpha 1) is a protein coding gene that enables protein serine/threonine kinase activity involving in several processes, including negative regulation of canonical Wnt signaling pathway; peptidyl-serine phosphorylation; and positive regulation of proteasomal ubiquitin-dependent protein catabolic process. Through phosphorylation of different substrate proteins, CK1a is able to activate, stabilize, inactivate, or destabilize the functions of these substrate proteins, thus regulating their functions.

In one embodiment of the disclosure, a compound, or pharmaceutically acceptable salt thereof, or pharmaceutical composition described herein is administered to a subject to degrade CK1a in the subject.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading CK1a in a subject suffering from a disorder, comprising administering to the subject a therapeutically effective amount of a compound described herein, or pharmaceutically acceptable salt thereof, or pharmaceutical composition described herein. In some embodiments, the compound binds to cereblon and a CK1a protein to induce ubiquitination and subsequent proteasomal degradation of the CK1α.

In certain embodiments, the compounds provided herein are degraders of a casein kinase 1. In certain embodiments, the compounds provided herein are degraders of casein kinase 1α (CK1α). In certain embodiments, the compounds provided herein are selective degraders of casein kinase 1α (CK1α). In certain embodiments, the compounds provided herein are degraders of human casein kinase 1α (CK1α). In certain embodiments, the compounds provided herein are selective degraders of human casein kinase 1α (CK1α).

Exemplary disorders that can be treated or prevented by the methods of the present disclosure include but are not limited to, cancer of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, non-Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AmoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading CK1a in a subject suffering from cancer (e.g., a cancer described herein), comprising administering to the subject a therapeutically effective amount of a compound described herein, or pharmaceutically acceptable salt thereof, or pharmaceutical composition described herein.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

In another aspect, described herein is a method of treating a liquid tumor (e.g., a liquid tumor described herein) in a subject in need thereof, comprising administering to the subject a therapeutically effective amount of a compound described herein, or pharmaceutically acceptable salt thereof, or pharmaceutical composition described herein.

In some embodiments, a method of the present disclosure further comprise administering to the subject an additional therapeutic agent.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃ 5, C₃₋₄, C₄₋₆, C₄ 5, and C₅₋₆ alkyl.

The term “alkyl” as used herein refers to a radical of a straight-chain or branched saturated hydrocarbon group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₉ alkyl”). In some embodiments, an alkyl group has 1 to 8 carbon atoms (“C₁₋₈ alkyl”). In some embodiments, an alkyl group has 1 to 7 carbon atoms (“C₁₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋ₛ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₆), 3-methyl-2-butanyl (C₅), tertiary amyl (C₅), and n-hexyl (C₆). Additional examples of alkyl groups include n-heptyl (C₇), n-octyl (C₈) and the like. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkenyl” as used herein refers to a radical of a straight-chain or branched hydrocarbon group having, one or more carbon-carbon double bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋ₛ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like.

The term “cycloalkyl” as used herein refers to a radical of a saturated or partially unsaturated cyclic hydrocarbon group having from 3 to 12 ring carbon atoms (“C₃₋₁₂ cycloalkyl”) and zero heteroatoms in the ring system. In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 3 to 6 ring carbon atoms (“C₃₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C), cyclohexenyl (C), cyclohexadienyl (C), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” as used herein refers to a radical of a saturated or partially unsaturated 3 to 10-membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “Spiro heterocyclyl,” or “spiro heterocycle” refers to a polycyclic heterocyclyl with rings connected through one common atom (called a spiro atom), wherein the rings have one or more heteroatoms selected from the group consisting of N, O, and S(O)m (wherein m is an integer of 0 to 2) as ring atoms.

The term “bridged-heterocycle” as used herein refers to a 4, 5, 6, 7, 8, 9, 10, 11, or 12-membered heterocycle as defined herein connected at two non-adjacent atoms of the 4, 5, 6, 7 or 8-membered heterocycle with one or more (e.g., 1 or 2) 3, 4, 5 or 6-membered heterocycles or (C₃-C₇)carbocycles as defined herein. Such bridged-heterocycles include bicyclic and tricyclic ring systems (e.g., 6-azabicyclo[3.1.1]heptane).

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR¹⁰⁰ where R¹⁰⁰ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical —CN.

The term “halogen” as used herein refers to F, Cl, Br, or I.

The term “oxo” as used herein refers to ═O.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²p, ³⁵S, ¹⁸F, and ³⁶Cl, respectively. For example, a compound of the disclosure may have one or more H atoms replaced with deuterium.

### Examples

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: ACN: acetonitrile; AIBN: azobisisobutyronitrile; BH₃·DMS: boron trifluoride methyl sulfide complex; BocNH₂: N-t-Butoxycarbonyl-amide; Boc₂O: di-tert-butyl dicarbonate; CDI: 1,1′-carbonyldiimidazole; CRBN: cereblon; DCM: dichloromethane; DIEA: N,N-diisopropylethylamine; DMF: N,N-dimethylformamide; eq: equivalents; DMSO: dimethyl sulfoxide; EDCl: 1-(3-dimethylaminopropyl)-3-ethylcarbodiimide hydrochloride; EI: electron ionization; ESI: electrospray ionization; h: hours; HATU: 0-(7-Azabenzotriazol-1-yl)-N,N,N,N-tetramethyluronium hexafluorophosphate; HOBt: 1-hydroxybenzotriazole; HPLC: high-performance liquid chromatography; LCMS: liquid chromatography mass spectrometry; MeCN: acetonitrile; MS: mass spectrometry; MTBE: tert-butyl methyl ether; NMR: nuclear magnetic resonance; Pd(dppf)Cl: [1,1-bis(diphenylphosphino) ferrocene]dichloropalladium (II); Py: pyridine; TEA: triethylamine; and t-BuONa: sodium tert-butoxide.

### Example 1. Synthesis of Compound 101

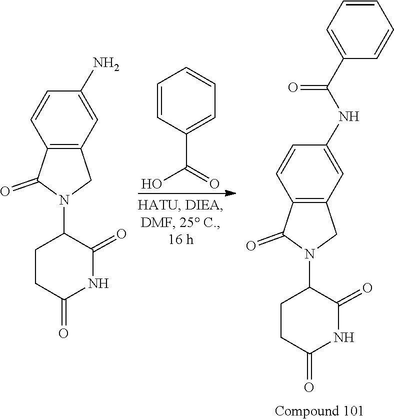 [SMILES: Nc1ccc2c(c1)CN(C1CCC(=O)NC1=O)C2=O ; O=C(O)c1ccccc1 ; O=C1CCC(N2Cc3cc(NC(=O)c4ccccc4)ccc3C2=O)C(=O)N1]

To a solution of benzoic acid (57.0 mg, 467 umol, 71.3 uL, 1.21 eq) in N,N-dimethyl formamide (1.50 mL) was added O-(7-Azabenzotriazol-1-yl)-N,N,N,N-tetramethyluronium hexafluorophosphate (220 mg, 579 umol, 1.50 eq) and N,N-diisopropylethylamine (100 mg, 775 umol, 135 μL, 2.01 eq). The mixture was stirred at 25° C. for 0.5 h. 3-(5-amino-1-oxoisoindolin-2-yl)piperidine-2,6-dione (100 mg, 386 umol, 1.00 eq) was added into the mixture and the mixture was stirred at 25° C. for 15.5 h. The mixture was adjusted pH=5 with formic acid and filtered which was purified by Prep-HPLC (column: Phenomenex luna C18 150*25 mm*10 um; mobile phase: [water(FA)-ACN]; B %: 15%-45%, 9 min) and further purified by Prep-HPLC (column: Phenomenex luna C18 150*25 mm*10 um; mobile phase: [water(FA)-ACN]; B %: 15%-45%, 9 min) and lyophilized to give N-(2-(2,6-dioxopiperidin-3-yl)-1-oxoisoindolin-5-yl)benzamide (Compound 101, 22.0 mg, 58.1 umol, 15% yield, 96% purity) as a white solid.

¹H NMR (400 MHz, DMSO-d₆) δ=11.08-10.86 (m, 1H), 10.56 (s, 1H), 8.15 (s, 1H), 7.98 (br d, J=7.8 Hz, 2H), 7.84 (br d, J=7.9 Hz, 1H), 7.71 (br d, J=8.0 Hz, 1H), 7.65-7.50 (m, 3H), 5.10 (br dd, J=5.1, 12.8 Hz, 1H), 4.52-4.41 (m, 1H), 4.38-4.29 (m, 1H), 2.98-2.87 (m, 1H), 2.64 (br d, J=16.5 Hz, 1H), 2.33 (br s, 1H), 2.05-1.97 (m, 1H). MS (ESI) m/z 364.0 [M+H]⁺.

### Example 2. Synthesis of Compound 102

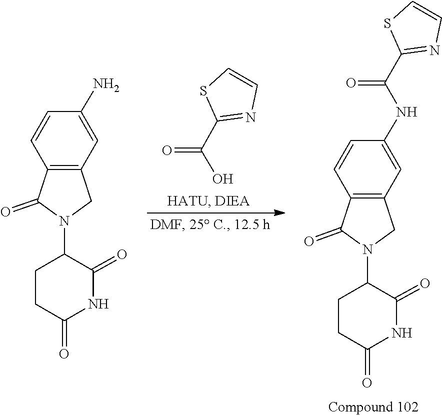 [SMILES: Nc1ccc2c(c1)CN(C1CCC(=O)NC1=O)C2=O ; O=C(O)c1nccs1 ; O=C1CCC(N2Cc3cc(NC(=O)c4nccs4)ccc3C2=O)C(=O)N1]

To a solution of thiazole-2-carboxylic acid (59.7 mg, 462 umol, 1.20 eq) in dimethyl formamide (1.00 mL) was added O-(7-Azabenzotriazol-1-yl)-N,N,N′,N′-tetramethyluronium Hexafluorophosphate (219 mg, 578 umol, 1.50 eq). The mixture was stirred at 25° C. for 0.5 h. The mixture was added 3-(5-amino-1-oxoisoindolin-2-yl)piperidine-2,6-dione (100 mg, 385 umol, 1.00 eq) and N,N-diisopropylethylamine (74.7 mg, 578 umol, 100 μL, 1.50 eq). The mixture was stirred was 25° C. for 12 h. The reaction mixture was concentrated under reduced pressure to give a residue. The crude product was dissolved in dimethyl formamide (2.00 mL) and purified by prep-HPLC (column: Phenomenex luna C18 150*25 mm*10 um; mobile phase: [water(FA)-ACN]; B %: 12%-42%, 10 min) and lyophilized to give N-(2-(2,6-dioxopiperidin-3-yl)-1-oxoisoindolin-5-yl)thiazole-2-carboxamide (Compound 102, 31.3 mg, 83.9 umol, 21% yield, 99% purity) as a yellow solid.

¹H NMR (400 MHz, DMSO-d₆) δ=11.11 (s, 1H), 10.98 (s, 1H), 8.20 (s, 1H), 8.18 (d, J=3.0 Hz, 1H), 8.14 (d, J=3.1 Hz, 1H), 7.96 (dd, J=1.4, 8.3 Hz, 1H), 7.72 (d, J=8.4 Hz, 1H), 5.10 (dd, J=5.1, 13.4 Hz, 1H), 4.51-4.45 (m, 1H), 4.38-4.27 (m, 1H), 2.97-2.87 (m, 1H), 2.62 (br d, J=1.8 Hz, 1H), 2.40 (br dd, J=4.6, 13.3 Hz, 1H), 2.04-1.97 (m, 1H). MS (ESI) m/z 371.0 [M+H]⁺.

### Example 3. Synthesis of Compound 103

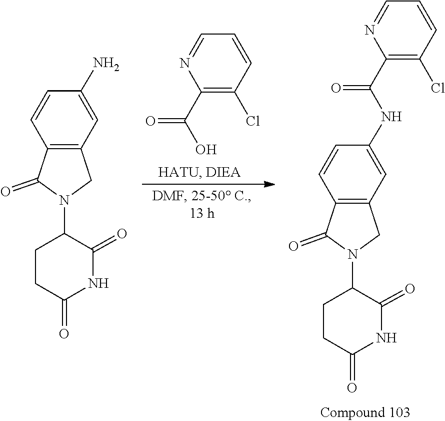 [SMILES: Nc1ccc2c(c1)CN(C1CCC(=O)NC1=O)C2=O ; O=C(O)c1ncccc1Cl ; O=C1CCC(N2Cc3cc(NC(=O)c4ncccc4Cl)ccc3C2=O)C(=O)N1]

A mixture of 3-chloropicolinic acid (72.9 mg, 463 umol, 1.20 eq) in N,N-dimethyl formamide (1.00 mL) was added diisopropylethylamine (150 mg, 1.16 mmol, 202 μL, 3.00 eq) and O-(7-azabenzotriazol-1-yl)-N,N,N,N-tetramethyluronium hexafluorophosphate (220 mg, 579 umol, 1.50 eq). Then 3-(5-amino-1-oxoisoindolin-2-yl)piperidine-2,6-dione (100 mg, 3856 umol, 1.00 eq) was added into the mixture. The mixture was stirred at 25° C. for 12 h. Then the mixture was stirred at 50° C. for another 1 h. The mixture was filtered and the filtrate was purified by Prep-HPLC (column: Phenomenex luna C18 150*25 mm*10 um; mobile phase:[water(FA)-ACN]; B %:13%-43%, 9 min). The desired fraction was collected and lyophilized to afford 3-chloro-N-(2-(2,6-dioxopiperidin-3-yl)-1-oxoisoindolin-5-yl) picolinamide (Compound 103, 45.0 mg, 110 umol, 28% yield, 97% purity) as a white solid.

¹H NMR (400 MHz, DMSO-d₆) δ=11.02 (s, 2H), 8.71-8.56 (m, 1H), 8.21-8.09 (m, 2H), 7.75 (q, J=8.3 Hz, 2H), 7.64 (dd, J=4.6, 8.3 Hz, 1H), 5.11 (dd, J=5.1, 13.3 Hz, 1H), 4.54-4.44 (m, 1H), 4.39-4.28 (m, 1H), 2.98-2.85 (m, 1H), 2.64-2.58 (m, 1H), 2.39 (br dd, J=4.2, 13.1 Hz, 1H), 2.06-1.96 (m, 1H). MS (ESI) m/z 399.0 [M+H]⁺.

### Example 4. Synthesis of Compound 104

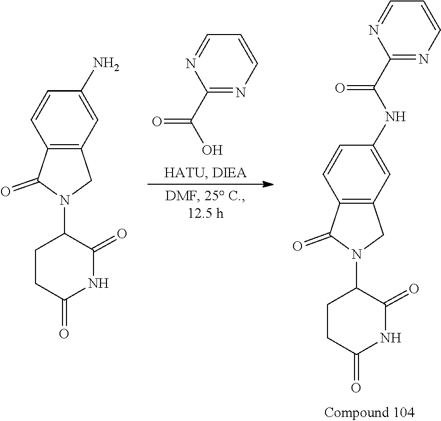 [SMILES: Nc1ccc2c(c1)CN(C1CCC(=O)NC1=O)C2=O ; O=C(O)c1ncccn1 ; O=C1CCC(N2Cc3cc(NC(=O)c4ncccn4)ccc3C2=O)C(=O)N1]

To a solution of pyrimidine-2-carboxylic acid (57.4 mg, 462 umol, 1.20 eq) in dimethyl formamide (1.00 mL) was added O-(7-Azabenzotriazol-1-yl)-N,N,N′,N′-tetramethyluronium Hexafluorophosphate (219 mg, 578 umol, 1.50 eq). The mixture was stirred at 25° C. for 0.5 h. The mixture was added N,N-diisopropylethylamine (74.7 mg, 578 umol, 100 μL, 1.50 eq) and 3-(5-amino-1-oxoisoindolin-2-yl)piperidine-2,6-dione (100 mg, 385 umol, 1.00 eq). The mixture was stirred at 25° C. for 12 h. The mixture was filtered to give a filter cake, which was triturated with water (10.0 mL), washed with water (1.00 mL) and dried to give N-(2-(2,6-dioxopiperidin-3-yl)-1-oxoisoindolin-5-yl)pyrimidine-2-carboxamide (Compound 104, 45.7 mg, 124 umol, 32% yield, 99% purity) as a yellow solid.

¹H NMR (400 MHz, DMSO-d₆) δ=11.06 (s, 1H), 10.98 (s, 1H), 9.06 (d, J=4.9 Hz, 2H), 8.24 (s, 1H), 8.01-7.90 (m, 1H), 7.77 (t, J=4.9 Hz, 1H), 7.73 (d, J=8.4 Hz, 1H), 5.11 (dd, J=5.1, 13.2 Hz, 1H), 4.53-4.45 (m, 1H), 4.39-4.31 (m, 1H), 2.98-2.86 (m, 1H), 2.61 (br d, J=17.0 Hz, 1H), 2.43-2.34 (m, 1H), 2.05-1.96 (m, 1H). MS (ESI) m/z 366.0 [M+H]⁺.

### Example 5. Synthesis of Compound 105

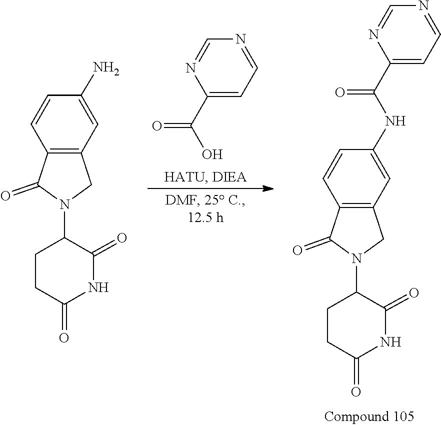 [SMILES: Nc1ccc2c(c1)CN(C1CCC(=O)NC1=O)C2=O ; O=C(O)c1ccncn1 ; O=C1CCC(N2Cc3cc(NC(=O)c4ccncn4)ccc3C2=O)C(=O)N1]

To a solution of pyrimidine-4-carboxylic acid (57.4 mg, 462 umol, 1.20 eq) in dimethyl formamide (2.00 mL) was added O-(7-azabenzotriazol-1-yl)-N,N,N′,N′-tetramethyluronium hexafluorophosphate (219 mg, 578 umol, 1.50 eq). The mixture was stirred at 25° C. for 0.5 h. 3-(5-amino-1-oxoisoindolin-2-yl)piperidine-2,6-dione (100 mg, 385 umol, 1.00 eq) and N,N-diisopropylethylamine (74.7 mg, 578 umol, 100 μL, 1.50 eq) were added to the reaction mixture. The mixture was stirred at 25° C. for 12 h. The mixture was filtered to give a filter cake, which was triturated with dimethyl formamide (2.00 mL) and filtered. The filter cake was washed with water (1.00 mL) and dried to give N-(2-(2,6-dioxopiperidin-3-yl)-1-oxoisoindolin-5-yl)pyrimidine-4-carboxamide (Compound 105, 10.0 mg, 24.4 umol, 6% yield) as a brown solid.

¹H NMR (400 MHz, DMSO-d₆) δ=11.15 (s, 1H), 10.99 (br s, 1H), 9.45 (d, J=1.3 Hz, 1H), 9.16 (d, J=5.0 Hz, 1H), 8.26 (s, 1H), 8.16 (dd, J=1.3, 5.1 Hz, 1H), 8.01 (dd, J=1.4, 8.4 Hz, 1H), 7.74 (d, J=8.3 Hz, 1H), 5.16-5.06 (m, 1H), 4.53-4.44 (m, 1H), 4.39-4.29 (m, 1H), 2.97-2.87 (m, 1H), 2.65-2.62 (m, 1H), 2.40 (br dd, J=4.4, 13.3 Hz, 1H), 2.06-1.97 (m, 1H). MS (ESI) m/z 366.0 [M+H]⁺.

### Example 6. Synthesis of Compound 106

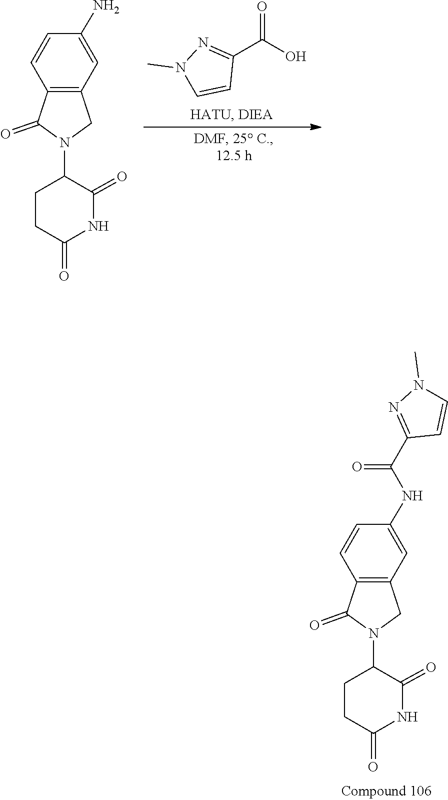

To a solution of 1-methyl-1H-pyrazole-3-carboxylic acid (116 mg, 925 umol, 1.20 eq) in dimethyl formamide (2.00 mL) was added O-(7-Azabenzotriazol-1-yl)-N,N,N′,N′-tetramethyluronium Hexafluorophosphate (439 mg, 1.16 mmol, 1.50 eq), N,N-diisopropylethylamine (199 mg, 1.54 mmol, 268 μL, 2.00 eq). The mixture was stirred at 25° C. for 0.5 h. The mixture was added 3-(5-amino-1-oxoisoindolin-2-yl)piperidine-2,6-dione (200 mg, 771 umol, 1.00 eq). The mixture was stirred at 25° C. for 12 h. The mixture was filtered to give a residue. The crude product was triturated with ethyl acetate (10.0 mL) and filtered. The filtered cake was concentrated under reduced pressure to give N-(2-(2,6-dioxopiperidin-3-yl)-1-oxoisoindolin-5-yl)-1-methyl-1H-pyrazole-3-carboxamide (Compound 106, 70.2 mg, 191 umol, 24% yield) as a white solid.

¹H NMR (400 MHz, DMSO-d₆) δ=10.97 (s, 1H), 10.37 (s, 1H), 8.20-8.12 (m, 1H), 7.98-7.84 (m, 2H), 7.67 (d, J=8.4 Hz, 1H), 6.79 (d, J=2.1 Hz, 1H), 5.09 (dd, J=5.0, 13.4 Hz, 1H), 4.49-4.41 (m, 1H), 4.35-4.27 (m, 1H), 3.98 (s, 3H), 2.97-2.85 (m, 1H), 2.60 (br d, J=17.8 Hz, 1H), 2.39 (br dd, J=4.3, 13.2 Hz, 1H), 2.04-1.96 (m, 1H). MS (ESI) m/z 368.1 [M+H].

### Example 7. Synthesis of Compound 107

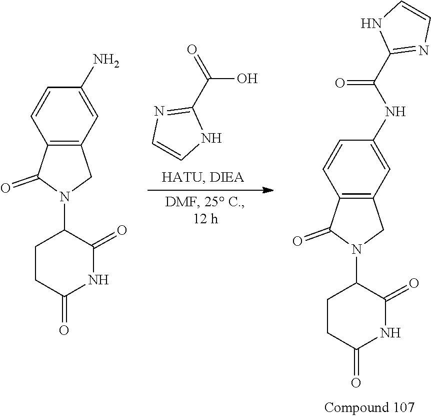 [SMILES: Nc1ccc2c(c1)CN(C1CCC(=O)NC1=O)C2=O ; O=C(O)c1ncc[nH]1 ; O=C1CCC(N2Cc3cc(NC(=O)c4ncc[nH]4)ccc3C2=O)C(=O)N1]

To a solution of 1H-imidazole-2-carboxylic acid (130 mg, 1.16 mmol, 1.51 eq) in N,N-dimethyl formamide (3.00 mL) was added O-(7-Azabenzotriazol-1-yl)-N,N,N,N-tetramethyl uronium hexafluorophosphate (440 mg, 1.16 mmol, 1.50 eq) and diisopropylethylamine (299 mg, 2.31 mmol, 403 μL, 3.00 eq), then 3-(5-amino-1-oxoisoindolin-2-yl)piperidine-2,6-dione (200 mg, 771 umol, 1.00 eq) was added into the mixture. The mixture was stirred at 25° C. for 12 h. The reaction mixture was concentrated under reduced pressure to give the crude product, which was purified by Prep-HPLC (column: Phenomenex luna C18 150*25 mm*10 um; mobile phase: [water(FA)-ACN]; B %: 1%-31%, 9 min). The desired fraction was collected and the aqueous solution was lyophilized to give the crude product, which was purified by Prep-HPLC (column: Phenomenex luna C18 150*25 mm*10 um; mobile phase: [water(FA)-ACN]; B %: 1%-31%, 9 min) and Prep-HPLC (column: Phenomenex luna C18 150*25 mm*10 um; mobile phase: [water(FA)-ACN]; B %: 1%-31%, 9 min). The desired fraction was collected and the aqueous solution was lyophilized to afford N-(2-(2,6-dioxopiperidin-3-yl)-1-oxoisoindolin-5-yl)-1H-imidazole-2-carboxamide (Compound 107, 14.86 mg, 41 umol, 5% yield, 98% purity) as a yellow solid.

¹H NMR (400 MHz, DMSO-d₆) δ=13.52-13.08 (m, 1H), 10.97 (br s, 1H), 10.71 (s, 1H), 8.18 (s, 1H), 7.96 (dd, J=1.6, 8.4 Hz, 1H), 7.69 (d, J=8.3 Hz, 1H), 7.41 (s, 1H), 7.18 (s, 1H), 5.10 (dd, J=5.1, 13.3 Hz, 1H), 4.50-4.42 (m, 1H), 4.35-4.28 (m, 1H), 2.97-2.87 (m, 1H), 2.64-2.57 (m, 1H), 2.39 (br dd, J=4.4, 12.9 Hz, 1H), 2.04-1.96 (m, 1H). MS (ESI) m/z 353.8 [M+H]⁺.

### Example 8. Synthesis of Compound 108

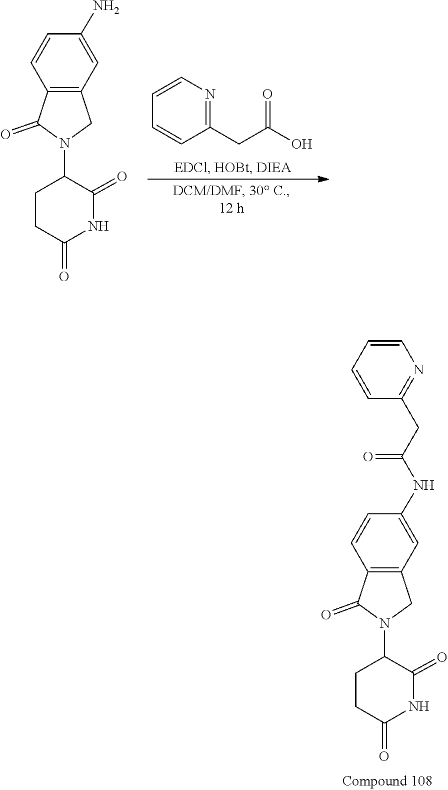 [SMILES: Nc1ccc2c(c1)CN(C1CCC(=O)NC1=O)C2=O ; O=C(O)Cc1ccccn1 ; O=C1CCC(N2Cc3cc(NC(=O)Cc4ccccn4)ccc3C2=O)C(=O)N1]

To a solution of 3-(5-amino-1-oxoisoindolin-2-yl)piperidine-2,6-dione (100 mg, 386 umol, 1.00 eq) and 2-(pyridin-2-yl)acetic acid (63.5 mg, 463 umol, 1.20 eq) in N,N-dimethyl formamide (0.900 mL) and dichloromethane (0.100 mL) was added diisopropylethylamine (150 mg, 1.16 mmol, 202 μL, 3.00 eq), 1-hydroxybenzotriazole (78.2 mg, 579 umol, 1.50 eq) and 1-(3-dimethylaminopropyl)-3-ethylcarbodiimide hydrochloride (111 mg, 579 umol, 1.50 eq). The mixture was stirred at 30° C. for 12 h. After being cooled to room temperature, the mixture was diluted with water (200 mL) and extracted with ethyl acetate (3×100 mL). The organic layers were collected and was washed with brine (50.0 mL), dried over anhydrous sodium sulfate and evaporated to dryness. The crude product was purified by Prep-HPLC (column: Phenomenex luna C18 150*25 mm*10 um; mobile phase: [water(FA)-ACN]; B %: 0%-22%, 9 min) and Prep-HPLC (column: Phenomenex luna C18 150*25 mm*10 um; mobile phase: [water(HCl)-ACN]; B %: 0%-19%, 2 min). The desired fraction was collected and lyophilized to afford N-(2-(2,6-dioxopiperidin-3-yl)-1-oxoisoindolin-5-yl)-2-(pyridine-2-yl)acetamide (Compound 108, 10.13 mg, 27.0 umol, 6% yield, 99% purity) as a white solid.

¹H NMR (400 MHz, DMSO-d₆) δ=11.02-10.87 (m, 2H), 8.82 (br d, J=5.4 Hz, 1H), 8.37 (br s, 1H), 7.99-7.88 (m, 2H), 7.86-7.77 (m, 1H), 7.73-7.62 (m, 2H), 5.08 (dd, J=5.1, 13.4 Hz, 1H), 4.46-4.40 (m, 1H), 4.32-4.24 (m, 3H), 2.94-2.87 (m, 1H), 2.61 (br d, J=2.0 Hz, 1H), 2.37 (br dd, J=4.5, 13.1 Hz, 1H), 2.04-1.95 (m, 1H). MS (ESI) m/z 379.1 [M+H]⁺.

### Example 9. Synthesis of Compound 109

### Step 1.

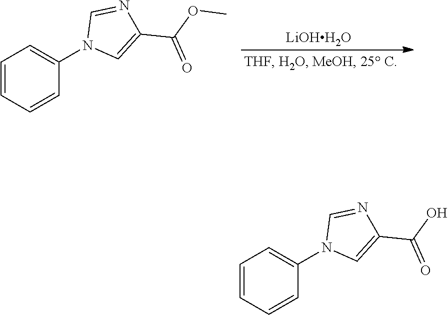

To a solution of methyl 1-phenyl-1H-imidazole-4-carboxylate (500 mg, 2.47 mmol, 1.10 eq) in tetrahydrofuran (3.00 mL), methanol (1.00 mL), water (1.00 mL) was added lithium hydroxide monohydrate (321 mg, 7.64 mmol, 3.40 eq). The mixture was stirred at 25° C. for 3 h. The mixture was concentrated under reduced pressure, then the cold water was added the mixture and acidified with 10% aqueous hydrochloric acid. Then the mixture was extracted with ethyl acetate (3×50 mL). The organic layers were collected and washed with brine (50.0 mL), dried over anhydrous sodium sulfate and evaporated to afford 1-phenyl-1H-imidazole-4-carboxylic acid (270 mg, crude) as a white solid.

¹H NMR (400 MHz, DMSO-d₆) δ=8.38 (s, 1H), 8.35 (s, 1H), 7.74 (d, J=7.9 Hz, 2H), 7.54 (t, J=7.8 Hz, 2H), 7.44-7.39 (m, 1H). MS (ESI) m/z. 189.0 [M+H]⁺.

### Step 2.

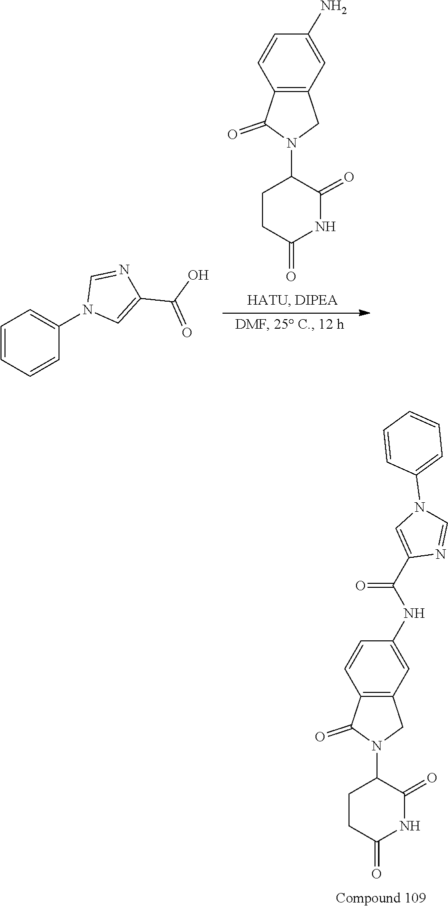 [SMILES: O=C(O)c1cn(-c2ccccc2)cn1 ; Nc1ccc2c(c1)CN(C1CCC(=O)NC1=O)C2=O ; O=C1CCC(N2Cc3cc(NC(=O)c4cn(-c5ccccc5)cn4)ccc3C2=O)C(=O)N1]

To a solution of 1-phenyl-1H-imidazole-4-carboxylic acid (200 mg, 1.06 mmol, 1.00 eq) in N,N-dimethyl formamide (2.00 mL) was added O-(7-Azabenzotriazol-1-yl)-N,N,N,N-tetramethyluronium hexafluorophosphate (606 mg, 1.59 mmol, 1.50 eq) and N,N-diisopropylethylamine (412 mg, 3.19 mmol, 555 μL, 3.00 eq). The mixture was stirred at 25° C. for 0.5 h. Then 3-(5-amino-1-oxoisoindolin-2-yl)piperidine-2,6-dione (275 mg, 1.06 mmol, 1.00 eq) was added into the mixture. The mixture was stirred at 25° C. for 12 h. The mixture was filtered. The filtrate was purified by prep-HPLC (column: Phenomenex luna C18 150*25 mm*10 um; mobile phase: [water(FA)-ACN]; B %: 17%-47%, 10 min). The desired fraction was collected and lyophilized to afford N-(2-(2,6-dioxopiperidin-3-yl)-1-oxoisoindolin-5-yl)-1-phenyl-1H-imidazole-4-carboxamide (Compound 109, 20.44 mg, 50.7 umol, 5% yield, 99% purity) as a yellow solid.

¹H NMR (400 MHz, DMSO-d₆) δ=10.98 (s, 1H), 10.33 (s, 1H), 8.50 (dd, J=1.3, 5.6 Hz, 2H), 8.21 (s, 1H), 7.96 (dd, J=1.4, 8.3 Hz, 1H), 7.80 (d, J=7.9 Hz, 2H), 7.69 (d, J=8.3 Hz, 1H), 7.57 (t, J=7.9 Hz, 2H), 7.47-7.41 (m, 1H), 5.10 (dd, J=5.0, 13.3 Hz, 1H), 4.51-4.43 (m, 1H), 4.38-4.27 (m, 1H), 2.96-2.88 (m, 1H), 2.58 (br s, 1H), 2.40 (br dd, J=4.4, 13.2 Hz, 1H), 2.04-1.98 (m, 1H). MS (ESI) m/z. 430.1 [M+H]⁺.

### Example 10. Synthesis of Compound 110

### Step 1.

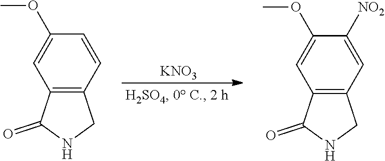

To a solution of 6-methoxyisoindolin-1-one (3.00 g, 18.4 mmol, 1.00 eq) in sulfuric acid (30.0 mL, 98% purity) was added potassium nitrate (1.86 g, 18.4 mmol, 1.00 eq) at 0° C., and the reaction mixture was stirred at 0° C. for 2 h. The mixture was treated with water (200 mL) and filtered. The filter cake was purified by Prep-HPLC (column: Welch Ultimate XB—CN 250*50*10 um; mobile phase: [Hexane-EtOH]; B %: 20%-55%, 20 min). The desired fraction was collected and concentrated under pressure to afford 6-methoxy-5-nitro-isoindolin-1-one (1.50 g, 7.21 mmol, 39% yield) as a gray solid.

¹H NMR (400 MHz, DMSO-d₆) δ=8.89 (br s, 1H), 8.07 (s, 1H), 7.52 (s, 1H), 4.37 (s, 2H), 3.98 (s, 3H).

### Step 2.

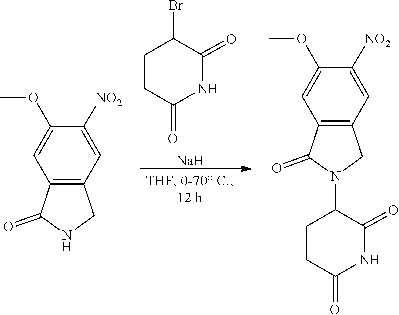

To a solution of 6-methoxy-5-nitroisoindolin-1-one (1.00 g, 4.80 mmol, 1.00 eq) in tetrahydrofuran (50.0 mL) was added sodium hydride (1.92 g, 48.0 mmol, 60% purity, 10.0 eq) at 0° C., and the mixture was stirred at 0° C. for 0.5 h under nitrogen atmosphere. Then 3-bromopiperidine-2,6-dione (4.61 g, 24.0 mmol, 5.00 eq) was added into the mixture. The mixture was stirred at 70° C. for 11.5 h under nitrogen atmosphere. The reaction mixture diluted with acetonitrile (10 mL), then the mixture was adjusted pH to 5-6 with formic acid and purified by reversed-phase HPLC (column: spherical C18, 20-45 um, 100 Å, SW 120, mobile phase: [water(0.1% Formic Acid)-ACN]; B %: 15%-80%, 30 min). The desired fraction was collected and lyophilized to afford the crude product, which was further purified by Prep-HPLC (column: Phenomenex luna C18 150*25 mm*10 μm; mobile phase: [water(FA)-ACN]; B %: 12%-42%, 9 min). The desired fraction was collected and lyophilized to afford 3-(6-methoxy-5-nitro-1-oxoisoindolin-2-yl)piperidine-2,6-dione (400 mg, 1.25 mmol, 26% yield) as a gray solid.

¹H NMR (400 MHz, DMSO-d₆) δ=11.02 (s, 1H), 8.14 (s, 1H), 7.62 (s, 1H), 5.15 (dd, J=5.1, 13.3 Hz, 1H), 4.50-4.44 (m, 1H), 4.39-4.32 (m, 1H), 4.01 (s, 3H), 2.96-2.86 (m, 1H), 2.61 (br d, J=16.6 Hz, 1H), 2.41 (br dd, J=4.5, 13.1 Hz, 1H), 2.06-2.00 (m, 1H). MS (ESI) m/z 320.0 [M+H]⁺.

### Step 3.

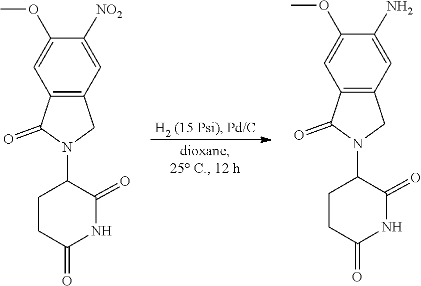 [SMILES: COc1cc2c(cc1[N+](=O)[O-])CN(C1CCC(=O)NC1=O)C2=O ; "H2 (15 Psi), Pd/C" ; COc1cc2c(cc1N)CN(C1CCC(=O)NC1=O)C2=O]

To a solution of 3-(6-methoxy-5-nitro-1-oxoisoindolin-2-yl)piperidine-2,6-dione (170 mg, 532 umol, 1.00 eq) in dioxane (15.0 mL) was added palladium on activated carbon (100 mg, 10% purity). The mixture was stirred at 25° C. for 12 h under hydrogen (15 Psi). The mixture was filtered to give the filtrate and concentrated under reduced pressure to afford 3-(5-amino-6-methoxy-1-oxoisoindolin-2-yl)piperidine-2,6-dione (150 mg, crude) as a gray solid. MS (ESI) m/z 296.0 [M+H]⁺.

### Step 4.

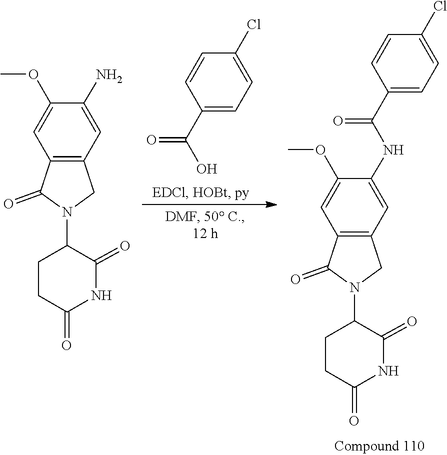 [SMILES: COc1cc2c(cc1N)CN(C1CCC(=O)NC1=O)C2=O ; O=C(O)c1ccc(Cl)cc1 ; "EDCl, HOBt, py" ; COc1cc2c(cc1NC(=O)c1ccc(Cl)cc1)CN(C1CCC(=O)NC1=O)C2=O]

To a solution of 3-(5-amino-6-methoxy-1-oxoisoindolin-2-yl)piperidine-2,6-dione (80.0 mg, 277 umol, 1.00 eq) and 4-chlorobenzoic acid (53.3 mg, 341 umol, 1.23 eq) in dimethyl formamide (2.00 mL) was added 1-(3-dimethylaminopropyl)-3-ethylcarbodiimide hydrochloride (80.0 mg, 417 umol, 1.51 eq), 1-hydroxybenzotriazole (45.0 mg, 333 umol, 1.20 eq) and pyridine (219 mg, 2.77 mmol, 223 μL, 10.0 eq). The mixture was stirred at 50° C. for 12 h. The reaction mixture was filtered to give the filtrate, which was purified by Prep-HPLC (column: Phenomenex luna C18 150*25 mm*10 um; mobile phase: [water(FA)-ACN]; B %: 25%-55%, 2 min). The desired fraction was collected and lyophilized to afford 4-chloro-N-(2-(2,6-dioxopiperidin-3-yl)-6-methoxy-1-oxoisoindolin-5-yl)benzamide (Compound 110, 9.46 mg, 22.0 umol, 8% yield, 99% purity) as an off-white solid.

¹H NMR (400 MHz, DMSO-d₆) δ=10.98 (s, 1H), 9.70 (s, 1H), 8.15 (s, 1H), 8.00 (d, J=8.5 Hz, 2H), 7.62 (d, J=8.6 Hz, 2H), 7.38 (s, 1H), 5.11 (dd, J=5.1, 13.4 Hz, 1H), 4.45-4.38 (m, 1H), 4.31-4.25 (m, 1H), 3.95 (s, 3H), 2.98-2.86 (m, 1H), 2.63-2.58 (m, 1H), 2.40 (dq, J=4.5, 13.3 Hz, 1H), 2.06-1.96 (m, 1H). MS (ESI) m/z 427.9 [M+H]⁺.

### Example 11. Synthesis of Compound 111

### Step 1.

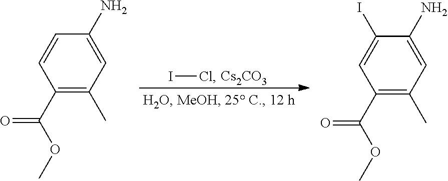

To a solution of methyl 4-amino-2-methylbenzoate (18.0 g, 108 mmol, 1.00 eq) in methanol (140 mL) was added cesium carbonate (35.5 g, 108 mmol, 1.00 eq) in water (100 mL). Then iodine monochloride (17.6 g, 108 mmol, 5.56 mL, 1.00 eq) in methanol (60 mL) was added under nitrogen. The mixture was stirred at 25° C. for 12 h. The reaction mixture was concentrated under reduced pressure to give a residue. The reaction mixture was diluted with water (300 mL) and exacted with ethyl acetate (2×300 mL). The organic phase was separated, washed with brine (100 mL), dried over sodium sulfate, filtered and concentrated under reduced pressure to give a residue. The residue was purified by reversed-phase HPLC (column: spherical C18, 20-45 um, 100 Å, SW 330, mobile phase: [water (0.1% formic acid)-acetonitrile]) to give methyl 4-amino-5-iodo-2-methylbenzoate (25.0 g, 62.7 mmol, 57% yield, 73% purity) as a brown solid.

¹H NMR (400 MHz, DMSO-d₆) δ=8.08 (s, 1H), 6.57 (s, 1H), 5.90 (br s, 2H), 3.73-3.71 (m, 3H), 2.37 (s, 3H). MS (ESI) m/z 291.9 [M+H]⁺.

### Step 2.

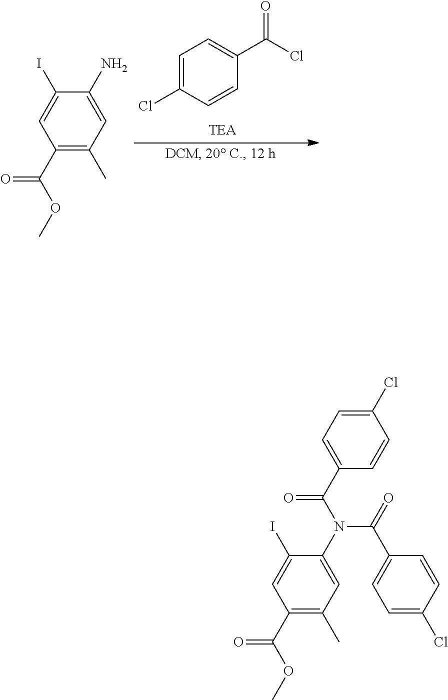 [SMILES: COC(=O)c1cc(I)c(N)cc1C ; O=C(Cl)c1ccc(Cl)cc1 ; COC(=O)c1cc(I)c(N(C(=O)c2ccc(Cl)cc2)C(=O)c2ccc(Cl)cc2)cc1C]

To a solution of methyl 4-amino-5-iodo-2-methylbenzoate (5.00 g, 17.1 mmol, 1.00 eq) in dichloromethane (50.0 mL) was added triethylamine (5.21 g, 51.5 mmol, 7.17 mL, 3.00 eq) and 4-chlorobenzoyl chloride (6.01 g, 34.3 mmol, 4.39 mL, 2.00 eq). The mixture was stirred at 20° C. for 12 h under nitrogen. The reaction mixture was concentrated under reduced pressure to give a residue. The residue was purified by reversed-phase HPLC (column: spherical C18, 20-45 um, 100 Å, SW 120, mobile phase: [water (0.1% formic acid)-acetonitrile]) to give methyl 4-(4-chloro-N-(4-chlorobenzoyl)benzamido)-5-iodo-2-methylbenzoate (7.00 g, 12.3 mmol, 71% yield) as a yellow solid.

### Step 3.

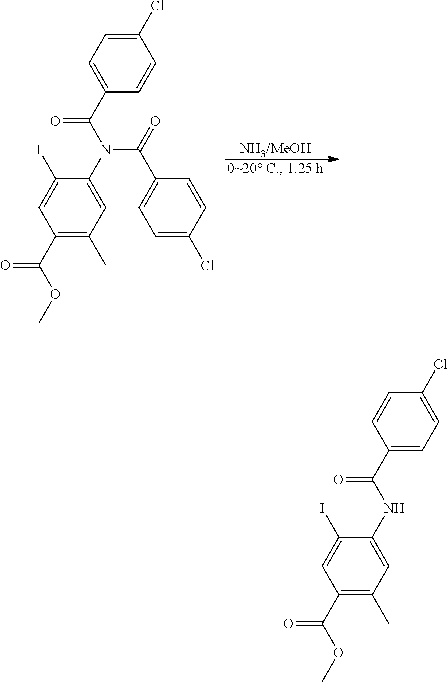 [SMILES: COC(=O)c1cc(I)c(N(C(=O)c2ccc(Cl)cc2)C(=O)c2ccc(Cl)cc2)cc1C ; COC(=O)c1cc(I)c(NC(=O)c2ccc(Cl)cc2)cc1C]

Ammonia was bubbled into methanol (70.0 mL) at 0° C. for 15 min. Then methyl 4-(4-chloro-N-(4-chlorobenzoyl)benzamido)-5-iodo-2-methylbenzoate (7.00 g, 12.3 mmol, 1.00 eq) was added into the mixture. The mixture was stirred at 20° C. for 1 h. The reaction mixture was concentrated under reduced pressure to give a residue. The crude product was purified by reversed-phase HPLC (column: spherical C18, 20-45 um, 100 Å, SW 330, mobile phase: [water(0.1% formic acid)-acetonitrile]) to give methyl 4-(4-chlorobenzamido)-5-iodo-2-methylbenzoate (5.00 g, 11.6 mmol, 94% yield) as a white solid.

¹H NMR (400 MHz, DMSO-d₆) δ=10.19 (br s, 1H), 8.30 (s, 1H), 8.03 (d, J=8.4 Hz, 2H), 7.65 (d, J=8.4 Hz, 2H), 7.50 (s, 1H), 3.87-3.84 (m, 3H), 2.50 (s, 3H). MS (ESI) m/z 429.7 [M+H]⁺.

### Step 4.

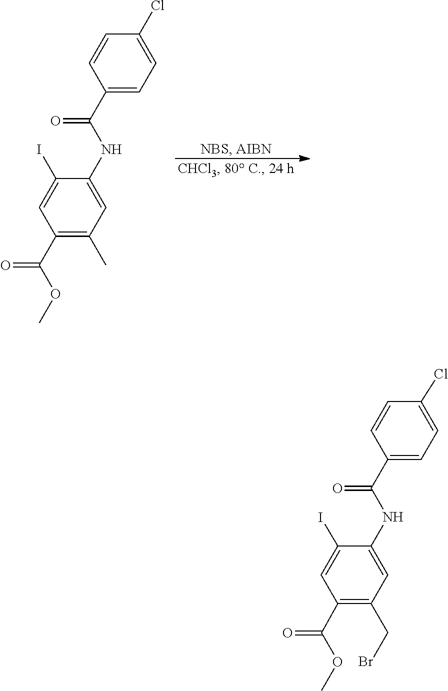 [SMILES: COC(=O)c1cc(I)c(NC(=O)c2ccc(Cl)cc2)cc1C ; COC(=O)c1cc(I)c(NC(=O)c2ccc(Cl)cc2)cc1CBr]

To a solution of methyl 4-(4-chlorobenzamido)-5-iodo-2-methylbenzoate (2.50 g, 5.82 mmol, 1.00 eq) in trichloromethane (120 mL) was added N-bromosuccinimide (3.11 g, 17.4 mmol, 3.00 eq) and azodiisobutyronitrile (1.91 g, 11.6 mmol, 2.00 eq). The mixture was stirred at 80° C. for 24 h. The reaction mixture was concentrated under reduced pressure to give a residue. The residue was purified by reversed-phase HPLC (column: spherical C18, 20-45 um, 100 Å, SW 330, mobile phase: [water (0.1% formic acid)-acetonitrile]) to give methyl 2-(bromomethyl)-4-(4-chlorobenzamido)-5-iodobenzoate (1.50 g, 2.95 mmol, 25% yield) as a yellow solid.

### Step 5.

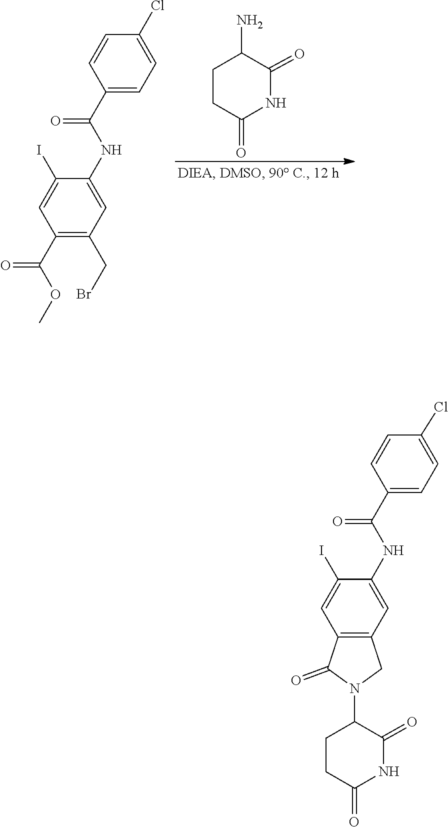 [SMILES: COC(=O)c1cc(I)c(NC(=O)c2ccc(Cl)cc2)cc1CBr ; NC1CCC(=O)NC1=O ; O=C1CCC(N2Cc3cc(NC(=O)c4ccc(Cl)cc4)c(I)cc3C2=O)C(=O)N1]

To a solution of methyl 2-(bromomethyl)-4-(4-chlorobenzamido)-5-iodobenzoate (1.50 g, 2.95 mmol, 1.00 eq) in dimethylsulfoxide (10.0 mL) was added 3-aminopiperidine-2,6-dione (485 mg, 2.95 mmol, 1.00 eq, hydrochloride) and N,N-diisopropylethylamine (1.14 g, 8.85 mmol, 1.54 mL, 3.00 eq) under nitrogen. The reaction mixture was stirred at 90° C. for 12 h. The mixture was filtered. The filtrate was purified by reversed-phase HPLC (column: spherical C18, 20-45 um, 100 Å, SW 330, mobile phase: [water (0.1% formic acid)-acetonitrile]) to give 4-chloro-N-(2-(2,6-dioxopiperidin-3-yl)-6-iodo-1-oxoisoindolin-5-yl)benzamide (600 mg, 1.15 mmol, 38% yield) as a purple solid.

¹H NMR (400 MHz, DMSO-d₆) δ=11.02 (s, 1H), 10.33-10.27 (m, 1H), 8.21 (s, 1H), 8.07-8.02 (m, 2H), 7.78 (s, 1H), 7.69-7.63 (m, 2H), 5.19-5.06 (m, 1H), 4.54-4.40 (m, 1H), 4.38-4.27 (m, 1H), 2.97-2.87 (m, 1H), 2.63-2.57 (m, 1H), 2.42-2.35 (m, 1H), 2.06-2.00 (m, 1H).

### Step 6.

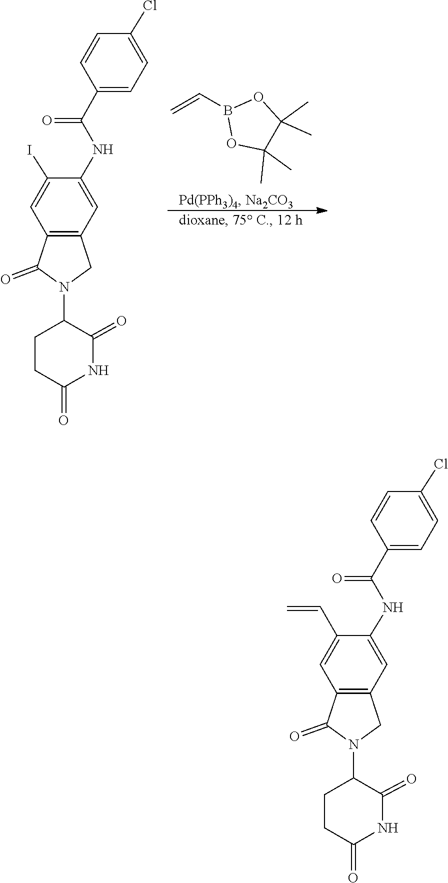

To a solution of 4-chloro-N-(2-(2,6-dioxopiperidin-3-yl)-6-iodo-1-oxoisoindolin-5-yl)benzamide (30.0 mg, 57.2 umol, 1.00 eq) in dioxane (2.00 mL) was added 4,4,5,5-tetramethyl-2-vinyl-1,3,2-dioxaborolane (26.4 mg, 171 umol, 29.1 uL, 3.00 eq), sodium carbonate (12.1 mg, 114 umol, 2.00 eq) and tetrakis(triphenylphosphine) palladium (6.62 mg, 5.73 umol, 0.100 eq) under nitrogen. The reaction mixture was stirred at 75° C. for 12 h. The reaction mixture was concentrated under reduced pressure to give a residue. The residue was purified by reversed-phase HPLC (column: spherical C18, 20-45 um, 100 Å, SW 120, mobile phase: [water (0.1% formic acid)-acetonitrile]) to give 4-chloro-N-(2-(2,6-dioxopiperidin-3-yl)-1-oxo-6-vinylisoindolin-5-yl)benzamide (15.0 mg, 35.3 umol, 6% yield) as a white solid.

¹H NMR (400 MHz, DMSO-d₆) δ=11.00 (br d, J=2.8 Hz, 1H), 10.38 (br s, 1H), 8.05-7.99 (m, 3H), 7.63 (br d, J=5.6 Hz, 3H), 7.05-6.84 (m, 1H), 5.97 (br d, J=17.6 Hz, 1H), 5.38 (br d, J=10.5 Hz, 1H), 5.14 (br d, J=9.1 Hz, 1H), 4.54-4.43 (m, 1H), 4.38-4.30 (m, 1H), 2.91 (br d, J=11.4 Hz, 1H), 2.62 (br s, 1H), 2.33 (br s, 1H), 1.98 (br d, J=3.5 Hz, 1H). MS (ESI) m/z 423.9 [M+H]⁺.

### Step 7.

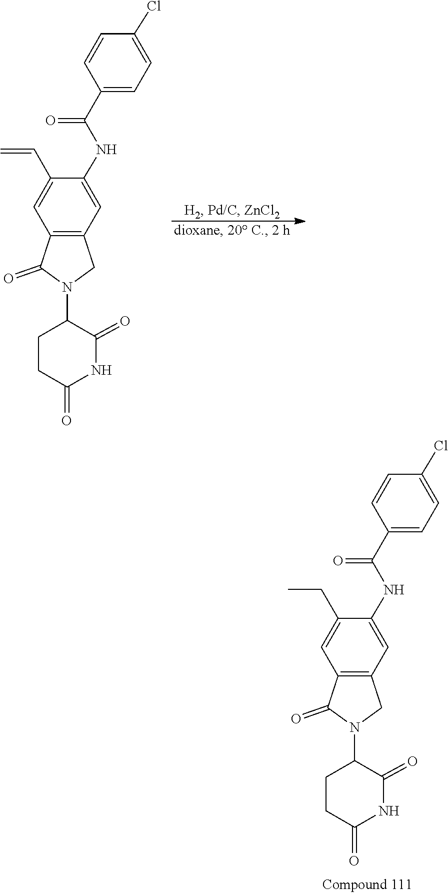 [SMILES: C=Cc1cc2c(cc1NC(=O)c1ccc(Cl)cc1)CN(C1CCC(=O)NC1=O)C2=O ; CCc1cc2c(cc1NC(=O)c1ccc(Cl)cc1)CN(C1CCC(=O)NC1=O)C2=O]

To a solution of 4-chloro-N-(2-(2,6-dioxopiperidin-3-yl)-1-oxo-6-vinylisoindolin-5-yl)benzamide (15.0 mg, 35.3 umol, 1.00 eq) in dioxane (10.0 mL) was added zinc dichloride (14.4 mg, 106 umol, 4.97 uL, 3.00 eq) and palladium on carbon (5.00 mg, 10% purity) under nitrogen. The reaction mixture was stirred at 20° C. for 2 h under hydrogen (15 Psi). The mixture was filtered. The filtrate was concentrated under reduced pressure to give a residue. The residue was purified by prep-HPLC (column: Phenomenex luna C18 150*25 mm*10 um; mobile phase: [water (formic acid)-acetonitrile]; B %: 25%-55%, 10 min) and lyophilized to afford 4-chloro-N-(2-(2,6-dioxopiperidin-3-yl)-6-ethyl-1-oxoisoindolin-5-yl)benzamide (Compound 111, 2.17 mg, 5.10 umol, 14% yield) as a white solid.

¹H NMR (400 MHz, DMSO-d₆) δ=11.06-10.92 (m, 1H), 10.15 (s, 1H), 8.53-8.37 (m, 0.2H), 8.07-7.94 (m, 2H), 7.65 (d, J=3.3 Hz, 2H), 7.64-7.61 (m, 2H), 5.12 (dd, J=5.1, 13.3 Hz, 1H), 4.48-4.40 (m, 1H), 4.36-4.28 (m, 1H), 2.97-2.87 (m, 1H), 2.75 (q, J=7.5 Hz, 2H), 2.63-2.59 (m, 1H), 2.43-2.35 (m, 1H), 2.05-1.98 (m, 1H), 1.17 (t, J=7.5 Hz, 3H). MS (ESI) m/z 426.1 [M+H]⁺.

### Example 12. Synthesis of Compound 112

### Step 1.

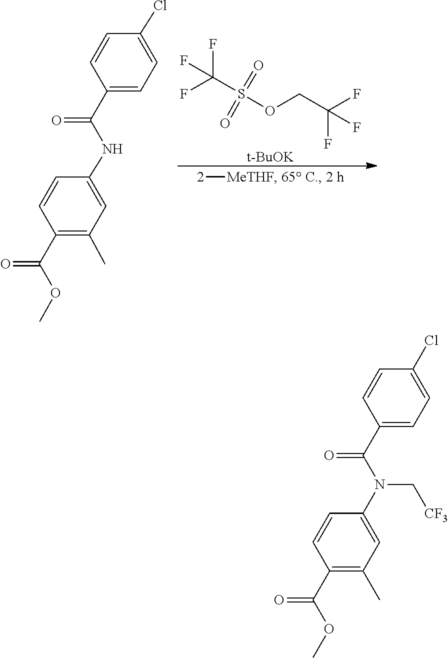

A solution of methyl 4-(4-chlorobenzamido)-2-methylbenzoate (4.00 g, 13.1 mmol, 1.00 eq) in 2-methyltetrahydrofuran (10.0 mL) was heated to 65° C. Then potassium tert-butoxide (1.48 g, 13.1 mmol, 1.00 eq), 2,2,2-trifluoroethyl trifluoromethanesulfonate (6.11 g, 26.3 mmol, 2.00 eq) was added and the mixture was stirred at 65° C. for 12 h. The solution was diluted with water (300 mL) and extracted with ethyl acetate (3×150 mL). The combined organic layers were washed with brine (3×30 mL), dried over anhydrous sodium sulfate, filtered and concentrated under reduced pressure to give a residue, which was purified by column chromatography (SiO₂, Petroleum ether/Ethyl acetate=50/1 to 25/1) and concentrated in vacuum to afford methyl 4-(4-chloro-N-(2,2,2-trifluoroethyl)benzamido)-2-methylbenzoate (2.30 g, 5.31 mmol, 40% yield, 89% purity) as a yellow solid.

¹H NMR (400 MHz, DMSO-d₆) δ=7.69 (d, J=8.4 Hz, 1H), 7.36-7.28 (m, 5H), 7.07 (dd, J=2.1, 8.3 Hz, 1H), 4.79 (q, J=9.3 Hz, 2H), 3.79 (s, 3H), 2.42 (s, 3H). MS (ESI) m/z 385.7 [M+H]⁺.

### Step 2.

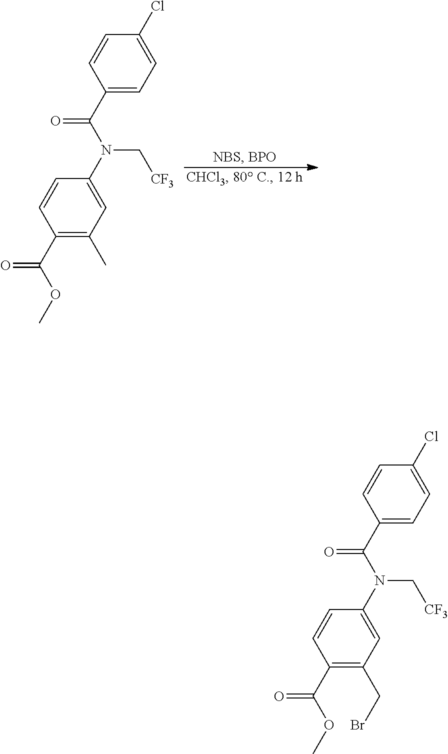 [SMILES: COC(=O)c1ccc(N(CC(F)(F)F)C(=O)c2ccc(Cl)cc2)cc1C ; COC(=O)c1ccc(N(CC(F)(F)F)C(=O)c2ccc(Cl)cc2)cc1CBr]

To a solution of methyl 4-(4-chloro-N-(2,2,2-trifluoroethyl)benzamido)-2-methylbenzoate (1.80 g, 4.67 mmol, 1.00 eq) in trichloromethane (50.0 mL) was added N-bromosuccinimide (2.49 g, 14.0 mmol, 3.00 eq) and benzoyl peroxide (1.13 g, 4.67 mmol, 1.00 eq). Then the solution was stirred at 80° C. for 12 h. The solution was diluted with water (300 mL) and extracted with ethyl acetate (3×150 mL). The combined organic layers were washed with brine (3×30 mL), dried over anhydrous sodium sulfate, filtered and concentrated under reduced pressure to give a residue, which was purified by column chromatography (SiO₂, Petroleum ether/Ethyl acetate=30/1 to 10/1) and concentrated in vacuum to afford methyl 2-(bromomethyl)-4-(4-chloro-N-(2,2,2-trifluoroethyl)benzamido)benzoate (2.30 g, crude) as a yellow solid.

¹H NMR (400 MHz, DMSO-d₆) δ=8.00-7.89 (m, 2H), 7.80 (dd, J=2.5, 8.4 Hz, 1H), 7.68-7.50 (m, 4H), 4.90-4.78 (m, 4H), 3.85 (s, 3H).

### Step 3.

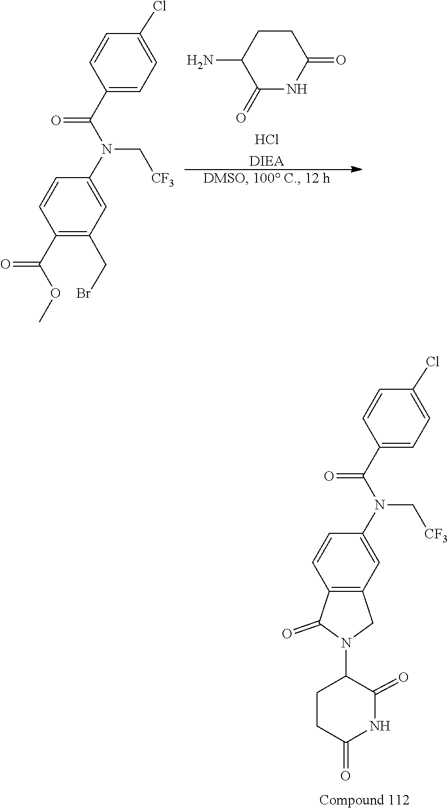 [SMILES: COC(=O)c1ccc(N(CC(F)(F)F)C(=O)c2ccc(Cl)cc2)cc1CBr ; NC1CCC(=O)NC1=O ; Cl ; O=C1CCC(N2Cc3cc(N(CC(F)(F)F)C(=O)c4ccc(Cl)cc4)ccc3C2=O)C(=O)N1]

To a solution of methyl 2-(bromomethyl)-4-(4-chloro-N-(2,2,2-trifluoroethyl)benzamido)benzoate (500 mg, 1.08 mmol, 1.00 eq) in dimethylsulfoxide (5.00 mL) was added 3-aminopiperidine-2,6-dione (177 mg, 1.08 mmol, 1.00 eq), N,N-diisopropylethylamine (417 mg, 3.23 mmol, 562 μL, 3.00 eq). Then the solution was stirred at 100° C. for 12 h. The solution was filtered. The filtrate was purified by prep-HPLC (column: Phenomenex luna C18 150*25 mm*10 um; mobile phase: [water(FA)-ACN]; B %: 31%-61%, 9 min) and lyophilized to afford 4-chloro-N-(2-(2,6-dioxopiperidin-3-yl)-1-oxoisoindolin-5-yl)-N-(2,2,2-trifluoroethyl) benzamide (Compound 112, 49.14 mg, 74 umol, 6% yield, 99% purity) as a white solid.

¹H NMR (400 MHz, DMSO-d₆) δ=10.98 (s, 1H), 7.65-7.57 (m, 2H), 7.34 (s, 4H), 7.29 (br d, J=8.3 Hz, 1H), 5.08 (dd, J=5.1, 13.3 Hz, 1H), 4.86-4.76 (m, 2H), 4.43-4.35 (m, 1H), 4.31-4.23 (m, 1H), 2.95-2.83 (m, 1H), 2.60 (br s, 1H), 2.43-2.34 (m, 1H), 1.99 (br dd, J=4.4, 6.8 Hz, 1H). MS (ESI) m/z 480.0 [M+H]⁺.

### Example 13. Synthesis of Compound 113

### Step 1.

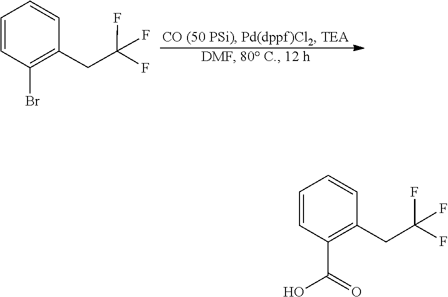 [SMILES: FC(F)(F)Cc1ccccc1Br ; O=C(O)c1ccccc1CC(F)(F)F]

To a mixture of 1-bromo-2-(2,2,2-trifluoroethyl)benzene (500 mg, 2.09 mmol, 1.00 eq), triethylamine (635 mg, 6.28 mmol, 873 μL, 3.00 eq) in dimethyformamide (10.0 mL) was added [1,1-bis(diphenylphosphino) ferrocene]dichloropalladium (II) (153 mg, 209 umol, 0.100 eq) in one portion. Then the mixture was stirred at 80° C. for 12 h under carbon monoxide (50 Psi). The mixture was diluted with water (50 mL) and extracted with ethyl acetate (3×50 mL). The combined organic layer was washed with brine (30 mL), dried over sodium sulfate, filtered and concentrated in vacuum. The residue was purified by reverse phase chromatography (column: spherical C18, 20-45 um, 100 Å, SW 80, mobile phase: [water (0.1% formic acid)-ACN). The desired fraction was collected and extracted with ethyl acetate (3×50 mL). The combined organic layer was washed with brine (30 mL), dried over sodium sulfate, filtered and concentrated in vacuum to afford 2-(2,2,2-trifluoroethyl)benzoic acid (60.0 mg, 294 umol, 14% yield) as a yellow solid.

¹H NMR (400 MHz, DMSO-d₆) δ=13.51-13.07 (m, 1H), 8.01-7.93 (m, 1H), 7.69-7.62 (m, 1H), 7.55 (dd, J=4.2, 7.1 Hz, 2H), 4.23 (q, J=11.4 Hz, 2H).

### Step 2

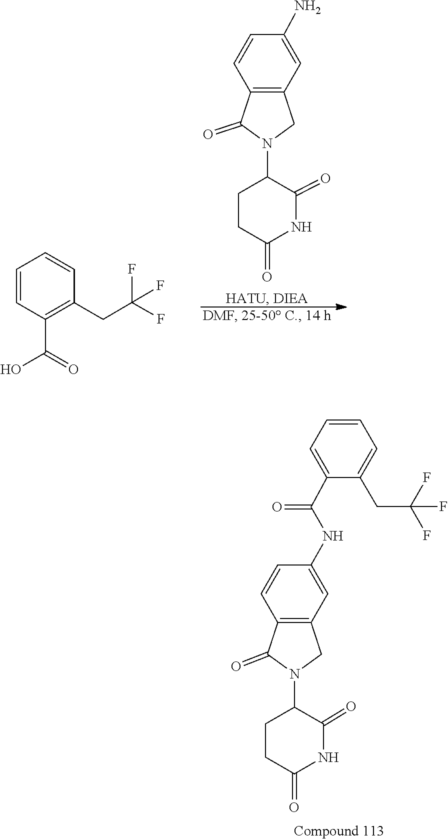 [SMILES: O=C(O)c1ccccc1CC(F)(F)F ; Nc1ccc2c(c1)CN(C1CCC(=O)NC1=O)C2=O ; O=C1CCC(N2Cc3cc(NC(=O)c4ccccc4CC(F)(F)F)ccc3C2=O)C(=O)N1]

To a mixture of 2-(2,2,2-trifluoroethyl)benzoic acid (47.2 mg, 231 umol, 1.20 eq), 3-(5-amino-1-oxo-isoindolin-2-yl)piperidine-2,6-dione (50.0 mg, 193 umol, 1 eq) and O-(7-azabenzotriazol-1-yl)-N,N,N,N-tetramethyluronium hexafluorophosphate (110 mg, 289 umol, 1.50 eq) in dimethyformamide (1.00 mL) was added N,N-diisopropylethylamine (74.8 mg, 579 umol, 101 μL, 3.00 eq) dropwise. The mixture was stirred at 25° C. for 12 h. Then the mixture was stirred at 50° C. for 2 h. The mixture was filtered. The filtrate was purified by reverse phase chromatography (column: spherical C18, 20-45 um, 100 Å, SW 40, mobile phase: [water (0.1% formic acid)-ACN) and lyophilized to afford N-(2-(2,6-dioxopiperidin-3-yl)-1-oxoisoindolin-5-yl)-2-(2,2,2-trifluoroethyl)benzamide (Compound 113, 5.80 mg, 12.4 umol, 6% yield, 95% purity) as a yellow solid.

¹H NMR (400 MHz, DMSO-d₆) δ=10.99 (s, 1H), 10.83 (s, 1H), 8.13 (s, 1H), 7.78-7.64 (m, 3H), 7.60-7.51 (m, 3H), 5.10 (dd, J=5.1, 13.3 Hz, 1H), 4.52-4.43 (m, 1H), 4.40-4.29 (m, 1H), 3.97 (q, J=11.4 Hz, 2H), 2.98-2.85 (m, 1H), 2.60 (dd, J=2.3, 15.4 Hz, 1H), 2.45-2.35 (m, 1H), 2.06-1.95 (m, 1H). MS (ESI) m/z 446.0 [M+H]⁺.

### Example 14. Synthesis of Compound 114

### Step 1.

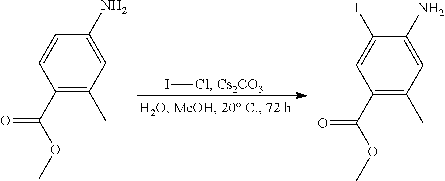

To a solution of methyl 4-amino-2-methylbenzoate (10.0 g, 60.5 mmol, 1.00 eq) in methanol (40.0 mL) was added a solution of cesium carbonate (19.7 g, 60.5 mmol, 1.00 eq) in water (30.0 mL). Then a solution of iodine chloride (14.7 g, 90.8 mmol, 4.64 mL, 1.50 eq) in methanol (40.0 mL) was added to the mixture at 0° C. slowly. The mixture was stirred at 20° C. for 72 h. The reaction mixture was concentrated to remove methanol. The residue was added with water (100 mL), extracted with ethyl acetate (3×150 mL). The combined organic layers was dried over anhydrous sodium sulfate, filtered and concentrated under reduced pressure to give a residue. The residue was purified by column chromatography (SiO₂, petroleum ether: ethyl acetate=1:0 to 10:1) and concentrated to afford methyl 4-amino-5-iodo-2-methylbenzoate (3.40 g, 9.34 mmol, 15% yield, 80% purity) as a yellow solid.

¹H NMR (400 MHz, DMSO-d₆) δ=8.09 (s, 1H), 6.58 (s, 1H), 6.06-5.54 (m, 2H), 3.72 (s, 3H), 2.38 (s, 3H).

### Step 2.

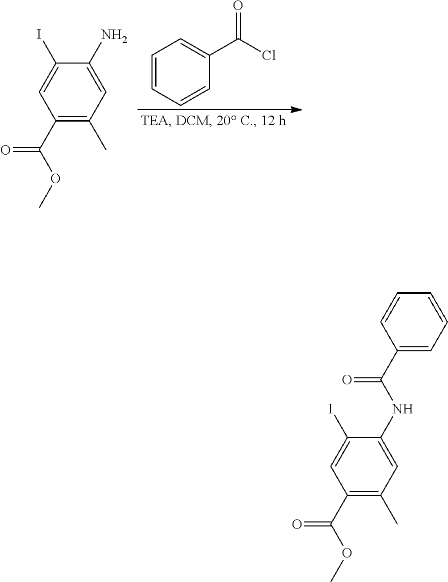

To a solution of methyl 4-amino-5-iodo-2-methylbenzoate (2.00 g, 6.87 mmol, 1.00 eq) in dichloromethane (20.0 mL) was added triethylamine (834 mg, 8.25 mmol, 1.15 mL, 1.20 eq) and benzoyl chloride (1.16 g, 8.25 mmol, 958 μL, 1.20 eq). The mixture was stirred at 20° C. for 12 h. The reaction mixture was concentrated under reduced pressure to give a residue. The residue was purified by reversed phase column chromatography (C18, 330 g; condition: water/acetonitrile=1/0 to 0/1, 0.1% formic acid) and concentrated to give a crude product, which was further purified by reversed phase column chromatography (C18, 330 g; condition: water/acetonitrile=1/0 to 0/1, 0.1% formic acid) and concentrated to afford methyl 4-benzamido-5-iodo-2-methylbenzoate (950 mg, 1.97 mmol, 29% yield, 82% purity) as a yellow solid.

¹H NMR (400 MHz, DMSO-d₆) δ=10.1 (s, 1H), 8.30 (s, 1H), 8.02-7.99 (m, 2H), 7.64-7.61 (m, 1H), 7.57 (d, J=7.8 Hz, 2H), 7.53 (s, 1H), 3.84 (s, 3H), 2.49-2.49 (m, 3H).

### Step 3.

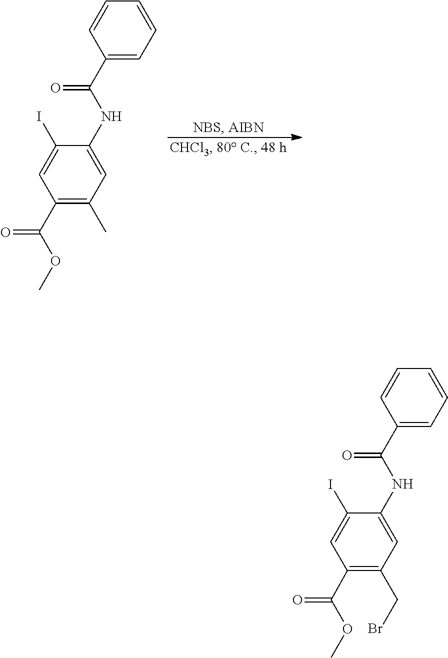 [SMILES: COC(=O)c1cc(I)c(NC(=O)c2ccccc2)cc1C ; COC(=O)c1cc(I)c(NC(=O)c2ccccc2)cc1CBr]

To a solution of methyl 4-benzamido-5-iodo-2-methylbenzoate (950 mg, 2.40 mmol, 1.00 eq) in trichloromethane (60.0 mL) was added N-bromosuccinimide (1.28 g, 7.21 mmol, 3.00 eq) and 2,2′-(diazene-1,2-diyl)bis(2-methylpropanenitrile) (789 mg, 4.81 mmol, 2.00 eq). The mixture was stirred at 80° C. for 48 h. The reaction mixture was concentrated under reduced pressure to give a residue. The residue was added water (15 mL), extracted with ethyl acetate (3×20 mL). The combined organic layers were washed with saturated sodium carbonate solution (20 mL), dried over anhydrous sodium sulfate, filtered and concentrated under reduced pressure to afford methyl 4-benzamido-2-(bromomethyl)-5-iodobenzoate (1.00 g, crude) as brown oil.

### Step 4.

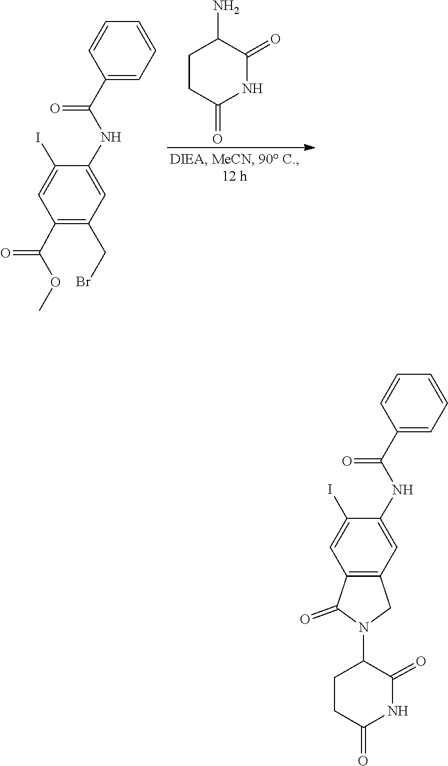

To a solution of methyl 4-benzamido-2-(bromomethyl)-5-iodobenzoate (1.00 g, 2.11 mmol, 1.00 eq) and 3-aminopiperidine-2,6-dione (347 mg, 2.11 mmol, 1.00 eq) in acetonitrile (30.0 mL) was added N, N-diisopropylethylamine (818 mg, 6.33 mmol, 1.10 mL, 3.00 eq). The mixture was stirred at 90° C. for 12 h. The reaction mixture was concentrated under reduced pressure to give a residue. The residue was purified by reversed phase column chromatography (C18, 120 g; condition: water/acetonitrile=1/0 to 0/1, 0.1% formic acid) and lyophilized to afford N-(2-(2,6-dioxopiperidin-3-yl)-6-iodo-1-oxoisoindolin-5-yl) benzamide (180 mg, 283 umol, 13% yield, 77% purity) as a gray solid. MS (ESI) m/z 489.9 [M+H]⁺.

### Step 5.

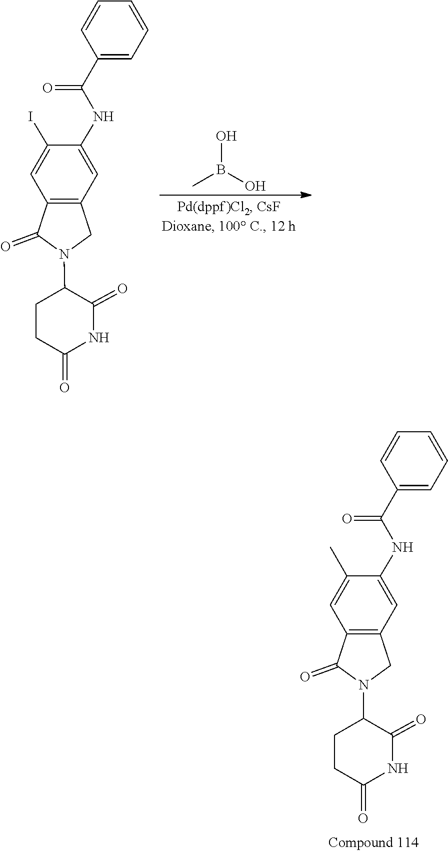

To a solution of N-(2-(2,6-dioxopiperidin-3-yl)-6-iodo-1-oxoisoindolin-5-yl)benzamide (40.0 mg, 61.3 umol, 75% purity, 1.00 eq) and methylboronic acid (11.0 mg, 184 umol, 3.00 eq) in dioxane (2.00 mL) was added [1,1-bis(diphenylphosphino)ferrocene]dichloropalladium(II) (4.49 mg, 6.13 umol, 0.100 eq) and cesium fluoride (32.6 mg, 215 umol, 7.91 uL, 3.50 eq). The mixture was stirred at 100° C. under nitrogen for 12 h. The reaction mixture was added dimethylsulfoxide (1.0 mL) and filtered. The filtrate was concentrated under reduced pressure to remove dioxane. The residue was purified by Prep-HPLC (column: Phenomenex luna C₁₈ 150*25 mm*10 um; mobile phase: [water (formic acid)-acetonitrile]; B %: 13%-43%, 10 min) and lyophilized to afford N-(2-(2,6-dioxopiperidin-3-yl)-6-methyl-1-oxoisoindolin-5-yl)benzamide (Compound 114, 9.98 mg, 26.2 umol, 43% yield, 99% purity) as a white solid.

¹H NMR (400 MHz, DMSO-d₆) δ=11.0 (s, 1H), 10.0 (s, 1H), 8.03-7.96 (m, 2H), 7.70 (s, 1H), 7.67-7.64 (m, 1H), 7.61 (d, J=7.3 Hz, 1H), 7.58-7.52 (m, 2H), 5.11 (dd, J=5.1, 13.3 Hz, 1H), 4.48-4.40 (m, 1H), 4.36-4.27 (m, 1H), 2.92 (br s, 1H), 2.68-2.63 (m, 1H), 2.39-2.31 (s, 3H), 2.06-1.98 (m, 1H). MS (ESI) m/z 378.1 [M+H]⁺.

### Example 15. Synthesis of Compound 115

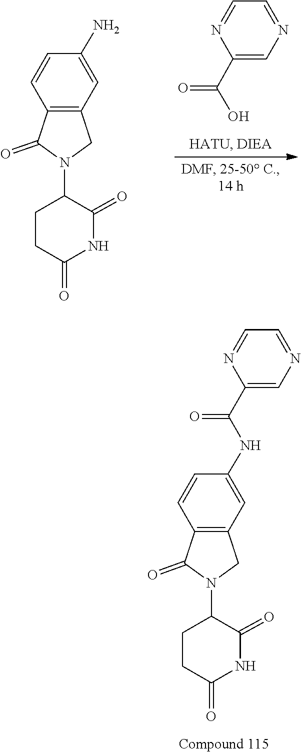 [SMILES: Nc1ccc2c(c1)CN(C1CCC(=O)NC1=O)C2=O ; O=C(O)c1cnccn1 ; O=C1CCC(N2Cc3cc(NC(=O)c4cnccn4)ccc3C2=O)C(=O)N1]

To a solution of pyrazine-2-carboxylic acid (200 mg, 1.61 mmol, 2.09 eq) in N,N-dimethyl formamide (3.00 mL) was added N,N-diisopropylethylamine (223 mg, 1.72 mmol, 300 μL, 2.23 eq) and O-(7-Azabenzotriazol-1-yl)-N,N,N,N-tetramethyl uronium hexafluorophosphate (440 mg, 1.16 mmol, 1.50 eq). Then 3-(5-amino-1-oxoisoindolin-2-yl)piperidine-2,6-dione (200 mg, 771 umol, 1.00 eq) was added into the mixture. The mixture was stirred at 25° C. for 2 h. Then the mixture was stirred at 50° C. for another 12 h. The mixture was filtered, the filtrate was purified by Prep-HPLC (column: Phenomenex luna C18 150*25 mm*10 um; mobile phase: [water(HCl)-ACN]; B %: 5%-35%, 10 min). The residual aqueous solution was lyophilized to give a white solid. The solid was triturated with dimethylsulfoxide (4 mL) and methyl tert-butyl ether (5 mL) to afford N-(2-(2,6-dioxopiperidin-3-yl)-1-oxoisoindolin-5-yl)pyrazine-2-carboxamide (26.0 mg, 70.4 umol, 9% yield, 99% purity) as a brown solid.

¹H NMR (400 MHz, DMSO-d₆) δ=11.05 (s, 1H), 10.98 (s, 1H), 9.32 (s, 1H), 8.96 (br d, J=2.1 Hz, 1H), 8.84 (br s, 1H), 8.25 (s, 1H), 7.99 (br d, J=7.9 Hz, 1H), 7.73 (br d, J=8.4 Hz, 1H), 5.11 (br dd, J=4.8, 13.3 Hz, 1H), 4.54-4.44 (m, 1H), 4.39-4.29 (m, 1H), 2.97-2.87 (m, 1H), 2.60 (br d, J=18.1 Hz, 1H), 2.40 (br dd, J=3.9, 13.3 Hz, 1H), 2.05-1.98 (m, 1H). MS (ESI) m/z 366.0 [M+H]⁺.

### Example 16. Synthesis of Compound 116

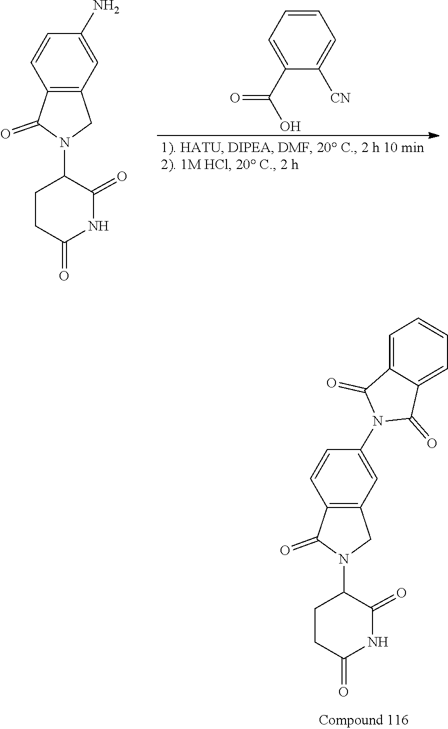 [SMILES: Nc1ccc2c(c1)CN(C1CCC(=O)NC1=O)C2=O ; N#Cc1ccccc1C(=O)O ; O=C1CCC(N2Cc3cc(N4C(=O)c5ccccc5C4=O)ccc3C2=O)C(=O)N1]

To a solution of 2-cyanobenzoic acid (136 mg, 925 umol, 1.20 eq), N,N-diisopropylethylamine (299 mg, 2.31 mmol, 403 μL, 3.00 eq) in dimethylformamide (3.00 mL) was added O-(7-azabenzotriazol-1-yl)-N,N,N,N-tetramethyluroniumhexafluorophosphate (586 mg, 1.54 mmol, 2.00 eq). The mixture was stirred at 20° C. for 10 min. Then 3-(5-amino-1-oxoisoindolin-2-yl)piperidine-2,6-dione (200 mg, 771 umol, 1.00 eq) was added. The mixture was stirred at 20° C. for 2 h. The mixture was filtered and the filtrate was purified by Prep-HPLC (column: Phenomenex C18 250*50 mm*10 um; mobile phase: [water(ammonium bicarbonate)-acetonitrile]; B %: 3%-33%, 8 min). The desired solution was collected and hydrochloric acid (1.00 M, 10.0 mL) was added. The mixture was stirred at 20° C. for 2 h. The mixture was lyophilized to give a residue, which was purified by Prep-HPLC (column: Phenomenex luna C18 150*25 mm*10 um; mobile phase: [water(hydrochloric acid)-acetonitrile]; B %: 19%-49%, 10 min) and lyophilized to afford 2′-(2,6-dioxopiperidin-3-yl)-[2,5′-biisoindoline]-1,1′,3-trione (Compound 116, 56.68 mg, 152 umol, 59% yield, 99% purity) as an off white solid.

¹H NMR (400 MHz, DMSO-d₆) δ=11.02 (s, 1H), 8.04-7.98 (m, 2H), 7.97-7.92 (m, 2H), 7.89 (d, J=8.0 Hz, 1H), 7.75-7.70 (m, 1H), 7.62 (d, J=8.0 Hz, 1H), 5.16 (dd, J=5.1, 13.4 Hz, 1H), 4.61-4.52 (m, 1H), 4.45-4.37 (m, 1H), 2.99-2.88 (m, 1H), 2.65-2.58 (m, 1H), 2.45-2.35 (m, 1H), 2.09-1.99 (m, 1H). MS (ESI) m/z 390.2 [M+H]⁺.

### Example 17. Synthesis of Compound 117

### Step 1.

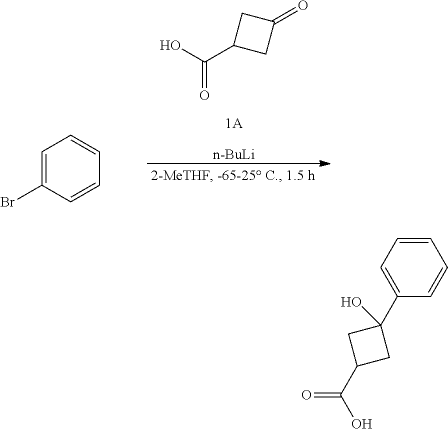

To a solution of bromobenzene (27.5 g, 17.0 mmol, 2.00 eq) in 2-methyltetrahydrofuran (400 mL) was added n-butyllithium (2.50 M, 70.1 mL, 2.00 eq) at −65° C. and the resulting mixture was stirred at −65° C. for 1 h under nitrogen atmosphere. Then 3-oxocyclobutanecarboxylic acid (10.0 g, 87.6 mmol, 1.00 eq) was added dropwise at −65° C. and the resulting mixture was stirred at 25° C. for 0.5 h. The reaction mixture was poured into saturated ammonium chloride solution (150 mL) and the resulting mixture was extracted with ethyl acetate (3×100 mL). The organic phase was collected. The combined organic layers were dried over sodium sulfate, filtered and concentrated under reduced pressure to give 3-hydroxy-3-phenylcyclobutanecarboxylic acid (19.0 g, crude) as a white solid.

¹H NMR (400 MHz, DMSO-d₆) δ=12.60-11.82 (m, 1H), 7.54-7.47 (m, 2H), 7.34 (t, J=7.6 Hz, 2H), 7.27-7.20 (m, 1H), 5.67 (br s, 1H), 2.73-2.65 (m, 1H), 2.64-2.57 (m, 2H), 2.56-2.51 (m, 1H), 2.47 (s, 1H).

### Step 2.

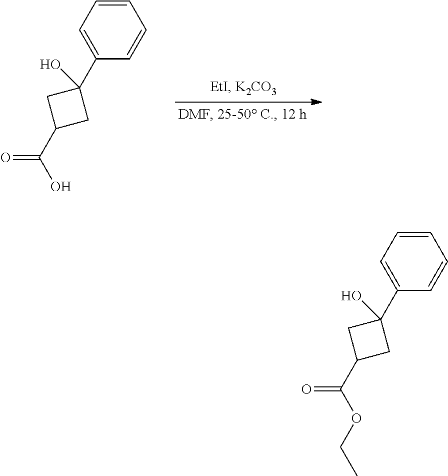 [SMILES: O=C(O)C1CC(O)(c2ccccc2)C1 ; CCOC(=O)C1CC(O)(c2ccccc2)C1]

To a solution of 3-hydroxy-3-phenylcyclobutanecarboxylic acid (7.00 g, 36.4 mmol, 1.00 eq) in dimethyl formamide (30.0 mL) was added potassium carbonate (6.04 g, 43.7 mmol, 1.20 eq) and iodoethane (11.4 g, 72.8 mmol, 2.00 eq) at 25° C. with stirring. The resulting mixture was stirred at 50° C. for 12 h. The reaction mixture was cooled to room temperature and filtered. The filtrate was concentrated under reduced pressure. The residue was purified by reversed phased column chromatography (C18, 330 g; condition: water/acetonitrile=1/0 to 0/1, 0.1% formic acid) to give ethyl 3-hydroxy-3-phenylcyclobutanecarboxylate (3.96 g, 18.0 mmol, 49% yield) as light yellow oil.

¹H NMR (400 MHz, DMSO-d₆) δ=7.51 (d, J=7.5 Hz, 2H), 7.35 (t, J=7.6 Hz, 2H), 7.28-7.20 (m, 1H), 5.69 (s, 1H), 4.09 (q, J=7.1 Hz, 2H), 2.76 (q, J=8.6 Hz, 1H), 2.68-2.60 (m, 2H), 2.53 (d, J=2.6 Hz, 2H), 1.22-1.15 (m, 3H).

### Step 3.

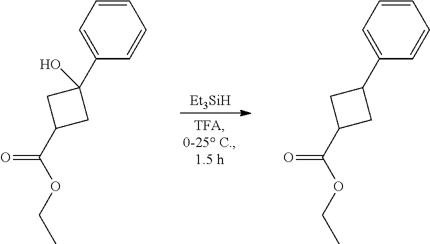 [SMILES: "Et3SiH" ; CCOC(=O)C1CC(O)(c2ccccc2)C1 ; CCOC(=O)C1CC(c2ccccc2)C1]

To a solution of ethyl 3-hydroxy-3-phenyl-cyclobutanecarboxylate (1.90 g, 8.63 mmol, 1.00 eq) in trifluoroacetic acid (10.0 mL) was added triethylsilane (6.02 g, 51.8 mmol, 8.27 mL, 6.00 eq) at 25° C. with stirring. The resulting mixture was stirred at 25° C. for 1.5 h. The reaction mixture was concentrated under reduced pressure. The residue was purified by reversed phase column chromatography (C18, 120; condition: water/acetonitrile=1/0 to 0/1, 0.1% formic acid) to give ethyl 3-phenylcyclobutanecarboxylate (1.36 g, 6.33 mmol, 73% yield, 95% purity) as light yellow oil.

¹H NMR (400 MHz, DMSO-d₆) δ=7.35-7.15 (m, 5H), 4.16-4.02 (m, 2H), 3.53-3.38 (m, 1H), 3.19-3.07 (m, 1H), 2.61-2.52 (m, 2H), 2.42-2.31 (m, 1H), 2.27-2.14 (m, 1H), 1.25-1.14 (m, 3H). MS (ESI) m/z 204.9 [M+H]⁺.

### Step 4.

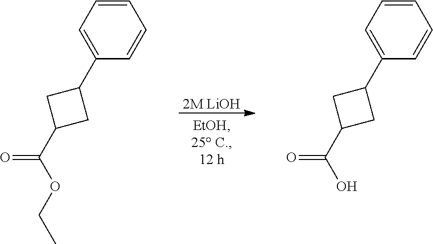

To a solution of ethyl 3-phenylcyclobutanecarboxylate (1.36 g, 6.66 mmol, 1.00 eq) in ethanol (6.00 mL) was added lithium hydroxide hydrate (2 M, 6.00 mL, 1.80 eq) at 25° C. with stirring. The resulting mixture was stirred at 25° C. for 12 h. The organic layer was removed and the pH of aqueous layer was adjusted to 2 with hydrochloric acid (12 M). The resulting mixture was poured into water (10 mL). The resulting mixture was extracted with ethyl acetate (3×30 mL) and the organic layers were collected. The combined organic layers were washed with brine (3×10 mL), dried over sodium sulfate, filtered and concentrated to give 3-phenylcyclobutanecarboxylic acid (1.00 g, 5.39 mmol, 81% yield, 95% purity) as light yellow oil.

¹H NMR (400 MHz, DMSO-d₆) δ=12.14 (br s, 1H), 7.34-7.15 (m, 5H), 3.47-3.38 (m, 1H), 3.11-3.00 (m, 1H), 2.58-2.51 (m, 2H), 2.38-2.28 (m, 1H), 2.25-2.15 (m, 1H).

### Step 5.

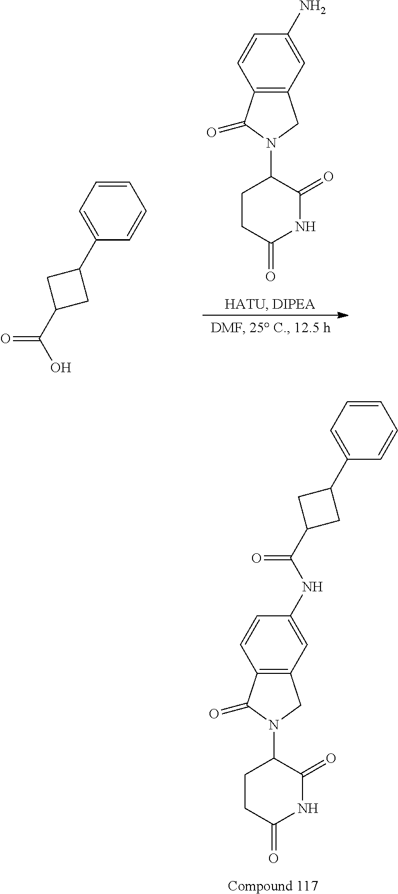 [SMILES: O=C(O)C1CC(c2ccccc2)C1 ; Nc1ccc2c(c1)CN(C1CCC(=O)NC1=O)C2=O ; "HATU, DIPEA" ; O=C1CCC(N2Cc3cc(NC(=O)C4CC(c5ccccc5)C4)ccc3C2=O)C(=O)N1 ; "Compound 117"]

A mixture of 3-phenylcyclobutanecarboxylic acid (92.7 mg, 526 umol, 1.00 eq), N,N-diisopropylethylamine (204 mg, 1.58 mmol, 3.00 eq) and O-(7-azabenzotriazol-1-yl)-N,N,N′,N′-tetramethyluronium hexafluorophosphate (400 mg, 1.05 mmol, 2.00 eq) in dimethyl formamide (4.00 mL) was stirred at 25° C. for 0.5 h. Then, 3-(5-amino-1-oxoisoindolin-2-yl)piperidine-2,6-dione (150 mg, 579 umol, 1.10 eq) was added and the resulting mixture was stirred at 25° C. for 12 h. The reaction mixture was poured into water (30 mL). The resulting mixture was extracted with ethyl acetate (3×30 mL) and the organic layers were collected. The combined organic layers were washed with brine (3×10 mL), dried over sodium sulfate, filtered and concentrated. The residue was purified by Prep-HPLC (column: Phenomenex Luna C18 150 mm×25 mm×10 μm; mobile phase: [water (FA)-ACN]; B %: 25%-58%, 9 min) and lyophilized to give N-(2-(2,6-dioxopiperidin-3-yl)-1-oxoisoindolin-5-yl)-3-phenylcyclobutanecarboxamide (Compound 117, 108 mg, 249 umol, 47% yield, 96% purity) as a white solid.

¹H NMR (400 MHz, DMSO-d₆) δ=10.97 (s, 1H), 10.23-10.18 (m, 1H), 8.06-8.00 (m, 1H), 7.69-7.59 (m, 2H), 7.36-7.25 (m, 4H), 7.24-7.16 (m, 1H), 5.14-5.02 (m, 1H), 4.47-4.38 (m, 1H), 4.33-4.25 (m, 1H), 3.53-3.42 (m, 1H), 3.30-3.20 (m, 1H), 2.97-2.85 (m, 1H), 2.69-2.62 (m, 1H), 2.59-2.53 (m, 2H), 2.42-2.26 (m, 3H), 1.99 (tdd, J=2.6, 5.0, 12.5 Hz, 1H).

¹H NMR (400 MHz, DMSO-d₆, T=80° C.) 6=10.69 (s, 1H), 9.94 (s, 1H), 8.02-7.96 (m, 1H), 7.66-7.62 (m, 2H), 7.37-7.25 (m, 4H), 7.24-7.14 (m, 1H), 5.08-4.99 (m, 1H), 4.48-4.28 (m, 2H), 3.56-3.43 (m, 1H), 3.39-3.20 (m, 1H), 2.95-2.82 (m, 1H), 2.72-2.63 (m, 1H), 2.63-2.55 (m, 2H), 2.42-2.31 (m, 3H), 2.11-1.97 (m, 1H). MS (ESI) m/z 418.0 [M+H]⁺.

### Example 18. Synthesis of Compound 118

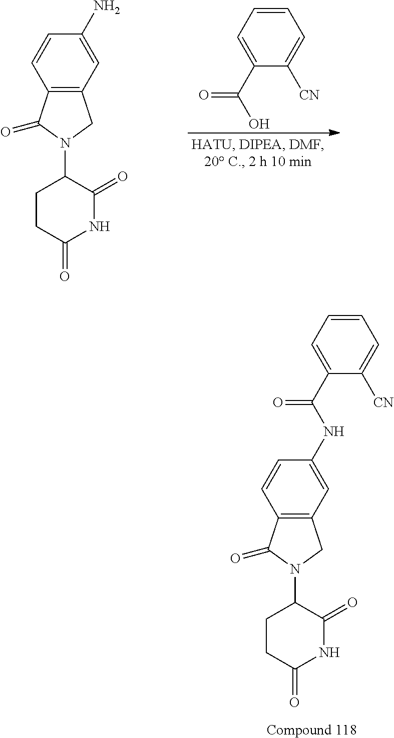

To a solution of 2-cyanobenzoic acid (136 mg, 925 umol, 1.20 eq), N,N-diisopropylethylamine (299 mg, 2.31 mmol, 403 μL, 3.00 eq) in dimethylformamide (3.00 mL) was added O-(7-azabenzotriazol-1-yl)-N,N,N,N-tetramethyluroniumhexafluorophosphate (586 mg, 1.54 mmol, 2.00 eq). The mixture was stirred at 20° C. for 10 min. Then 3-(5-amino-1-oxoisoindolin-2-yl)piperidine-2,6-dione (200 mg, 771 umol, 1.00 eq) was added and the mixture was stirred at 20° C. for 2 h. The pH of the mixture was adjusted to 5-6 with formic acid and filtered. The filtrate was purified by Prep-HPLC (column: Phenomenex luna C18 150*25 mm*10 um; mobile phase: [water(hydrochloric acid)-acetonitrile]; B %: 12%-42%, 10 min) and lyophilized to afford 2-cyano-N-(2-(2,6-dioxopiperidin-3-yl)-1-oxoisoindolin-5-yl)benzamide (Compound 118, 63.04 mg, 160 umol, 20% yield, 96% purity) as a yellow solid.

¹H NMR (400 MHz, DMSO-d₆) δ=10.98 (s, 2H), 8.13 (s, 1H), 8.04-8.00 (m, 1H), 7.97 (d, J=7.3 Hz, 1H), 7.88 (dt, J=1.1, 7.7 Hz, 1H), 7.79-7.72 (m, 3H), 5.11 (dd, J=5.1, 13.3 Hz, 1H), 4.53-4.46 (d, J=17.6 Hz, 1H), 4.38-4.32 (d, J=17.6 Hz, 1H), 2.99-2.87 (m, 1H), 2.61 (m, 1H), 2.44-2.31 (m, 1H), 2.08-1.97 (m, 1H). MS (ESI) m/z 388.9 [M+H]⁺.

### Example 19. Synthesis of Compound 119

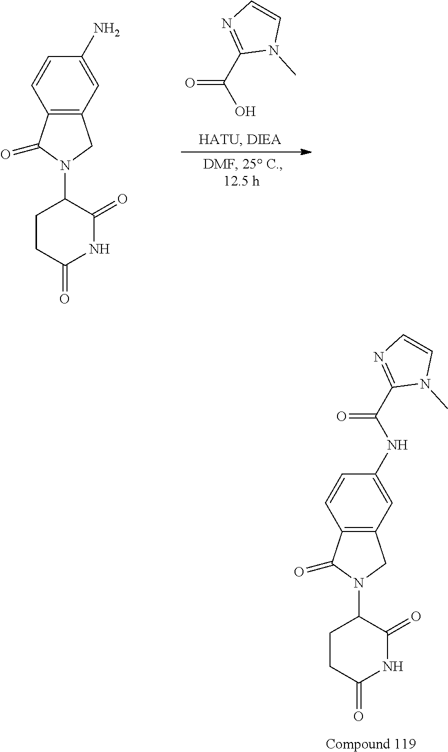

To a solution of 1-methyl-1H-imidazole-2-carboxylic acid (58.4 mg, 463 umol, 1.20 eq) in dimethyl formamide (2.00 mL) was added O-(7-Azabenzotriazol-1-yl)-N,N,N′,N′-tetramethyluronium Hexafluorophosphate (220 mg, 579 umol, 1.50 eq), N,N-diisopropylethylamine (99.7 mg, 771 umol, 134 μL, 2.00 eq). The mixture was stirred at 25° C. for 0.5 h. Then 3-(5-amino-1-oxoisoindolin-2-yl)piperidine-2,6-dione (100 mg, 386 umol, 1.00 eq) was added into the mixture. The mixture was stirred at 25° C. for 12 h. The mixture was filtered and the filter cake was triturated with water (8.00 mL) at 25° C. for 10 min and filtered. The filter cake was dried over in vacuum to afford N-(2-(2,6-dioxopiperidin-3-yl)-1-oxoisoindolin-5-yl)-1-methyl-1H-imidazole-2-carboxamide (Compound 119, 35.2 mg, 94.9 umol, 24% yield, 99% purity) as an off-white solid.

¹H NMR (400 MHz, DMSO-d₆) δ=10.99 (s, 1H), 10.68 (s, 1H), 8.19 (s, 1H), 7.91 (dd, J=1.4, 8.3 Hz, 1H), 7.68 (d, J=8.4 Hz, 1H), 7.48 (s, 1H), 7.11 (s, 1H), 5.10 (dd, J=5.0, 13.3 Hz, 1H), 4.51-4.38 (m, 1H), 4.36-4.25 (m, 1H), 4.01 (s, 3H), 2.95-2.87 (m, 1H), 2.62-2.57 (m, 1H), 2.39 (br dd, J=4.4, 13.2 Hz, 1H), 2.04-1.95 (m, 1H). MS (ESI) m/z 368.1 [M+H]⁺.

### Example 20. Synthesis of Compound 120

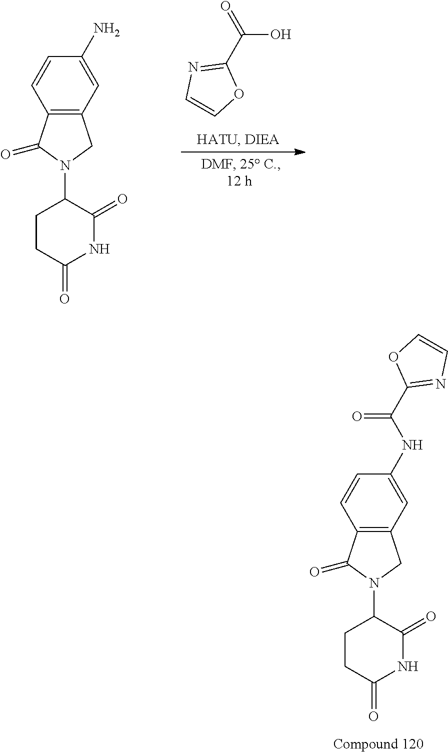

To a solution of oxazole-2-carboxylic acid (87.2 mg, 771 umol, 1.00 eq) in dimethyl formamide (1.00 mL) was added N,N-diisopropylethylamine (149 mg, 1.16 mmol, 201 μL, 1.50 eq), 0-(7-Azabenzotriazol-1-yl)-N,N,N′,N′-tetramethyluronium hexafluorophosphate (439 mg, 1.16 mmol, 1.50 eq) and 3-(5-amino-1-oxoisoindolin-2-yl)piperidine-2,6-dione (200 mg, 771 umol, 1.00 eq). The mixture was stirred at 25° C. for 12 h. The mixture was filtered, then the filter cake was triturated with water (10 mL) and filtered. The filter cake was dried to give N-(2-(2,6-dioxopiperidin-3-yl)-1-oxoisoindolin-5-yl)oxazole-2-carboxamide (Compound 120, 36.8 mg, 103 umol, 13% yield) as a white solid.

¹H NMR (400 MHz, DMSO-d₆) δ=11.16 (s, 1H), 10.98 (s, 1H), 8.43 (s, 1H), 8.15 (s, 1H), 7.91 (dd, J=1.6, 8.3 Hz, 1H), 7.72 (d, J=8.4 Hz, 1H), 7.58 (s, 1H), 5.10 (dd, J=5.1, 13.3 Hz, 1H), 4.54-4.44 (m, 1H), 4.38-4.28 (m, 1H), 2.96-2.88 (m, 1H), 2.60 (br d, J=18.0 Hz, 1H), 2.43-2.33 (m, 1H), 2.05-1.97 (m, 1H). MS (ESI) m/z 355.0 [M+H]⁺.

### Example 21. Synthesis of Compound 121

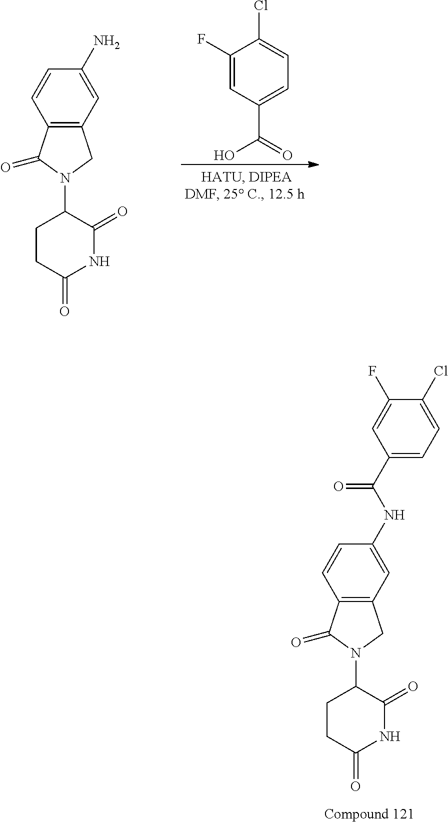

A mixture of 4-chloro-3-fluorobenzoic acid (224 mg, 1.29 mmol, 1.00 eq), N,N-diisopropylethylamine (499 mg, 3.86 mmol, 3.00 eq) and O-(7-Azabenzotriazol-1-yl)-N,N,N′,N′-tetramethyluronium hexafluorophosphate (978 mg, 2.57 mmol, 2.00 eq) in dimethyl formamide (6.00 mL) was stirred at 25° C. for 0.5 h. Then, 3-(5-amino-1-oxoisoindolin-2-yl)piperidine-2,6-dione (400 mg, 1.54 mmol, 1.20 eq) was added and the resulting mixture was stirred at 25° C. for 12 h. The reaction mixture was poured into water (50 mL) and ethyl acetate (50 mL). The resulting mixture was filtered and the filter cake was triturated with dimethyl formamide (6 mL) and filtered. The filter cake was collected and lyophilized to afford 4-chloro-N-(2-(2,6-dioxopiperidin-3-yl)-1-oxoisoindolin-5-yl)-3-fluorobenzamide (Compound 121, 177 mg, 409 umol, 32% yield, 96% purity) as a white solid.

¹H NMR (400 MHz, DMSO-d₆) δ=10.98 (s, 1H), 10.71 (s, 1H), 8.13 (s, 1H), 8.04 (d, J=9.8 Hz, 1H), 7.97-7.66 (m, 4H), 5.10 (dd, J=4.2, 12.8 Hz, 1H), 4.54-4.41 (m, 1H), 4.40-4.28 (m, 1H), 2.98-2.85 (m, 1H), 2.61 (br d, J=16.8 Hz, 1H), 2.40 (br d, J=12.8 Hz, 1H), 2.08-1.93 (m, 1H). MS (ESI) m/z 415.9 [M+H]⁺.

### Example 22. Synthesis of Compound 122

### Step 1.

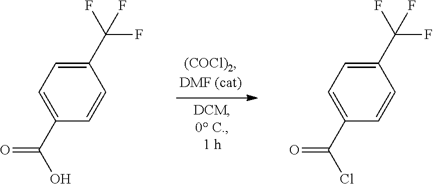 [SMILES: O=C(O)c1ccc(C(F)(F)F)cc1 ; O=C(Cl)c1ccc(C(F)(F)F)cc1]

To a solution of 4-(trifluoromethyl)benzoic acid (500 mg, 2.63 mmol, 1.00 eq) in dichloromethane (3.00 mL) was added oxalyl dichloride (334 mg, 2.63 mmol, 230 μL, 1.00 eq) and N,N-dimethyl formamide (96.1 mg, 1.31 mmol, 101 uL, 0.500 eq). The mixture was stirred at 0° C. for 1 h. The mixture was concentrated under reduced pressure to afford 4-(trifluoromethyl)benzoyl chloride (540 mg, crude) as yellow oil.

### Step 2.

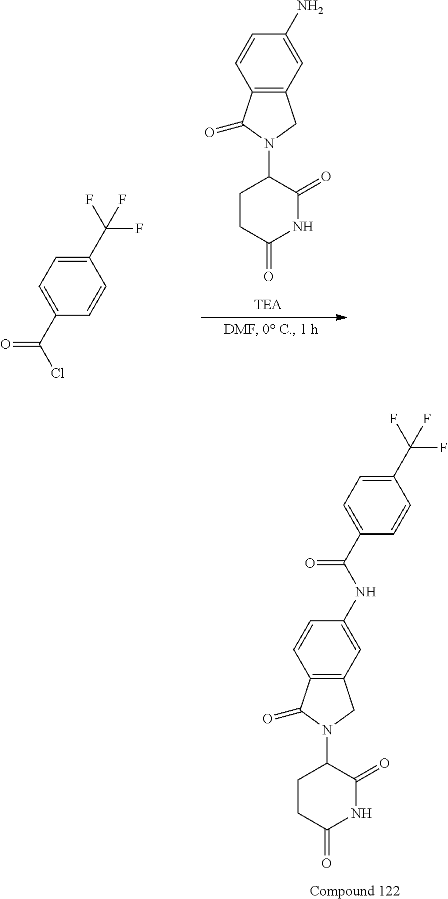

To a solution 3-(5-amino-1-oxoisoindolin-2-yl)piperidine-2,6-dione (300 mg, 1.16 mmol, 1.00 eq) in N,N-dimethyl formamide (2.00 mL) was added trimethylamine (351 mg, 3.47 mmol, 483 μL, 3.00 eq) and 4-(trifluoromethyl)benzoyl chloride (241 mg, 1.16 mmol, 172 μL, 1.00 eq). The mixture was stirred at 0° C. for 1 h. The mixture was adjusted to pH=6 with formic acid, then the mixture was filtered and the filtrate was purified by Prep-HPLC (column: Phenomenex luna C18 150 mm*25 mm*10 um; mobile phase: [water (FA)-ACN]; B %: 27%-57%, min). The desired fraction was collected and the aqueous solution was lyophilized to afford N-(2-(2,6-dioxopiperidin-3-yl)-1-oxoisoindolin-5-yl)-4-(trifluoromethyl)benzamide (Compound 122, 40.0 mg, 92.7 umol, 8% yield) as a white solid.

¹H NMR (400 MHz, DMSO-d₆) δ=10.99 (s, 1H), 10.78 (s, 1H), 8.19-8.14 (m, 3H), 7.94 (d, J=8.3 Hz, 2H), 7.84 (dd, J=1.7, 8.4 Hz, 1H), 7.74 (d, J=8.3 Hz, 1H), 5.11 (dd, J=5.0, 13.3 Hz, 1H), 4.52-4.45 (m, 1H), 4.38-4.31 (m, 1H), 2.97-2.87 (m, 1H), 2.63-2.57 (m, 1H), 2.43-2.37 (m, 1H), 2.08-1.97 (m, 1H). MS (ESI) m/z 432.0 [M+H]⁺.

### Example 23. Synthesis of Compound 123

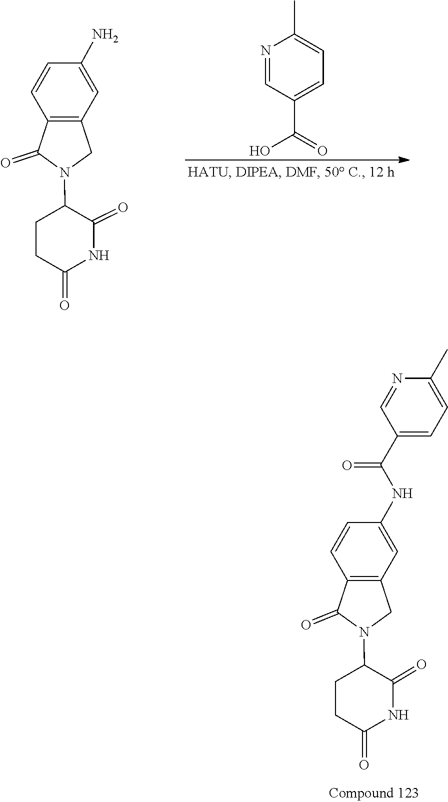

To a solution of 3-(5-amino-1-oxo-isoindolin-2-yl)piperidine-2,6-dione (100 mg, 385 umol, 1.00 eq) in dimethylformamide (0.500 mL) was added nicotinic acid (71.2 mg, 578 umol, 48.4 uL, 1.50 eq), O-(7-azabenzotriazol-1-yl)-N,N,N,N-tetramethyluroniumhexafluorophosphate (219 mg, 578 umol, 1.50 eq) and N,N-diisopropylethylamine (149 mg, 1.16 mmol, 201 μL, 3.00 eq). The reaction mixture was stirred at 50° C. for 12 h. The reaction mixture was added formic acid (2.00 mL) and filtered. The filtrate was purified by Prep-HPLC (column: Phenomenex luna C18 150 mm*25 mm*10 um; mobile phase: [water(formic acid)-acetonitrile]; B %: 1%-30%, 10.5 min) and lyophilized to give N-(2-(2,6-dioxopiperidin-3-yl)-1-oxoisoindolin-5-yl)-6-methylnicotinamide (Compound 123, 32.4 mg, 75.4 umol, 19% yield, 99% purity, formate) as a white solid.

¹H NMR (400 MHz, DMSO-d₆) δ=10.99 (s, 1H), 10.65 (s, 1H), 9.03 (d, J=2.1 Hz, 1H), 8.23 (dd, J=2.4, 8.1 Hz, 1H), 8.14 (s, 1H), 7.83 (dd, J=1.6, 8.4 Hz, 1H), 7.73 (d, J=8.3 Hz, 1H), 7.45 (d, J=8.0 Hz, 1H), 5.11 (dd, J=5.1, 13.4 Hz, 1H), 4.54-4.43 (m, 1H), 4.39-4.30 (m, 1H), 2.99-2.87 (m, 1H), 2.63 (br d, J=2.5 Hz, 1H), 2.57 (s, 3H), 2.42 (dt, J=4.4, 13.1 Hz, 1H), 2.07-1.97 (m, 1H). MS (ESI) m/z 379.0 [M+H].

### Example 24. Synthesis of Compound 124

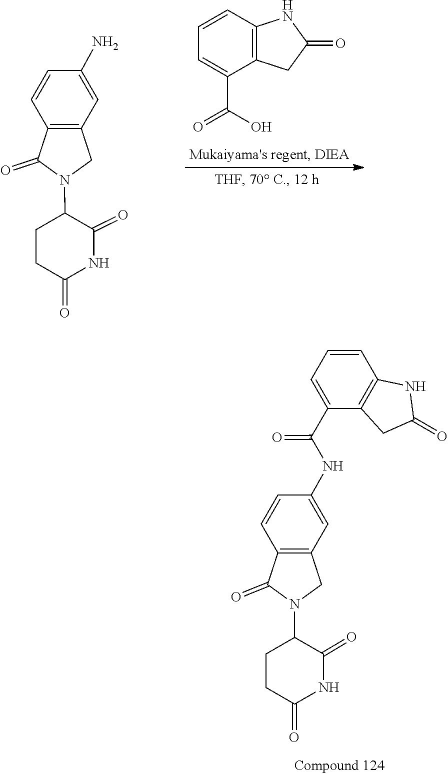 [SMILES: Nc1ccc2c(c1)CN(C1CCC(=O)NC1=O)C2=O ; O=C1Cc2c(cccc2C(=O)O)N1 ; O=C1CCC(N2Cc3cc(NC(=O)c4cccc5c4CC(=O)N5)ccc3C2=O)C(=O)N1]

A mixture of 3-(5-amino-1-oxoisoindolin-2-yl)piperidine-2,6-dione (400 mg, 1.54 mmol, 1.00 eq), 2-oxoindoline-4-carboxylic acid (273 mg, 1.54 mmol, 1.00 eq), N,N-diisopropylethylamine (598 mg, 4.63 mmol, 3.00 eq) and 2-chloro-1-methylpyridin-1-ium iodide (591 mg, 2.31 mmol, 1.50 eq) in tetrahydrofuran (10.0 mL) was stirred at 70° C. for 12 h. The reaction mixture was cooled to room temperature and poured into water (50 mL). The resulting mixture was extracted with ethyl acetate (3×50 mL) and the organic layers were collected. The combined organic layers were washed with brine (3×10 mL), dried over sodium sulfate, filtered and concentrated under reduced pressure. The residue was purified by prep-HPLC (column: Phenomenex Luna C18 150 mm×25 mm×10 μm; mobile phase: [water (FA)-ACN]; B %: 8%-38%, 9 min) and prep-HPLC (column: Welch X timate C18 150 mm×25 mm×5 μm; mobile phase: [water (NH3·H2O)-ACN]; B %: 1%-31%, 11 min). The residue was further purified by prep-HPLC (column: Phenomenex Luna C18 150 mm×25 mm×10 μm; mobile phase: [water (FA)-ACN]; B %: 7%-37%, 10.5 min). The desired fraction was collected and lyophilized to afford N-(2-(2,6-dioxopiperidin-3-yl)-1-oxoisoindolin-5-yl)-2-oxoindoline-4-carboxamide (Compound 124, 5.53 mg, 13.8 umol, 1% yield, 96% purity) as a white solid.

¹H NMR (400 MHz, DMSO-d₆) δ=10.98 (s, 1H), 10.56 (s, 1H), 10.50 (s, 1H), 8.13 (s, 1H), 7.80 (d, J=8.8 Hz, 1H), 7.71 (d, J=8.4 Hz, 1H), 7.47-7.41 (m, 1H), 7.39-7.31 (m, 1H), 7.02 (d, J=7.4 Hz, 1H), 5.10 (dd, J=5.0, 13.2 Hz, 1H), 4.51-4.28 (m, 2H), 3.72 (s, 2H), 2.97-2.86 (m, 1H), 2.63-2.59 (m, 1H), 2.41-2.35 (m, 1H), 2.06-1.97 (m, 1H). MS (ESI) m/z 419.0 [M+H]⁺.

### Example 25. Synthesis of Compound 125

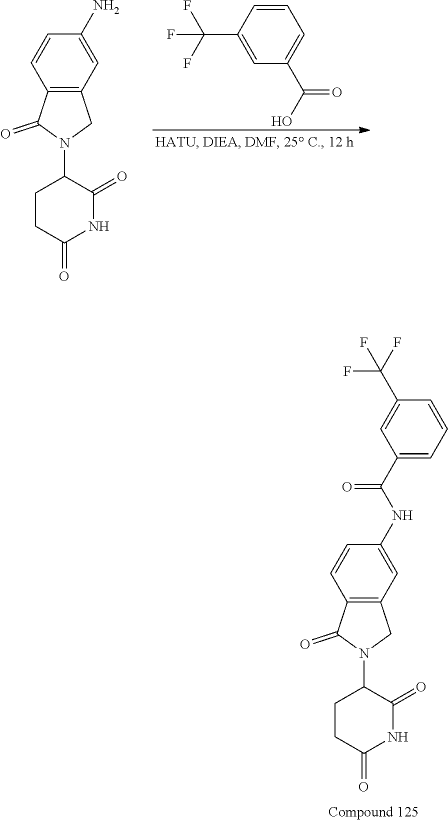 [SMILES: Nc1ccc2c(c1)CN(C1CCC(=O)NC1=O)C2=O ; O=C(O)c1cccc(C(F)(F)F)c1 ; O=C1CCC(N2Cc3cc(NC(=O)c4cccc(C(F)(F)F)c4)ccc3C2=O)C(=O)N1]

To a solution of 3-(5-amino-1-oxoisoindolin-2-yl)piperidine-2,6-dione (100 mg, 385 umol, 1.00 eq) and 3-(trifluoromethyl)benzoic acid (88.0 mg, 462 umol, 1.20 eq) in dimethyformamide (2.00 mL) was added O-(7-azabenzotriazol-1-yl)-N,N,N,N-tetramethyluronium hexafluorophosphate (219 mg, 578 umol, 1.50 eq) and N,N-diisopropylethylamine (149 mg, 1.16 mmol, 201 μL, 3.00 eq). The mixture was stirred at 25° C. for 12 h. The mixture was adjusted pH to 5-6 with formic acid (0.1 mL) and filtered. The filtrate was purified by Prep-HPLC (column: Phenomenex luna C18 150*25 mm*10 um; mobile phase: [water(formic acid)-acetonitrile]; B %: 27%-57%, 9 min) and lyophilized to afford N-(2-(2,6-dioxopiperidin-3-yl)-1-oxoisoindolin-5-yl)-3-(trifluoromethyl)benzamide (Compound 125, 15.22 mg, 46.3 umol, 12% yield, 99% purity) as a white solid.

¹H NMR (400 MHz, DMSO-d₆) δ=10.98 (s, 1H), 10.76 (s, 1H), 8.37-8.23 (m, 2H), 8.13 (s, 1H), 8.00 (br d, J=7.8 Hz, 1H), 7.88-7.78 (m, 2H), 7.77-7.69 (m, 1H), 5.18-5.04 (m, 1H), 4.56-4.42 (m, 1H), 4.41-4.26 (m, 1H), 2.95-2.90 (m, 1H), 2.61 (m, 1H), 2.38 (br s, 1H), 2.05-1.99 (m, 1H). MS (ESI) m/z. 431.9 [M+H]⁺.

### Example 26. Synthesis of Compound 126

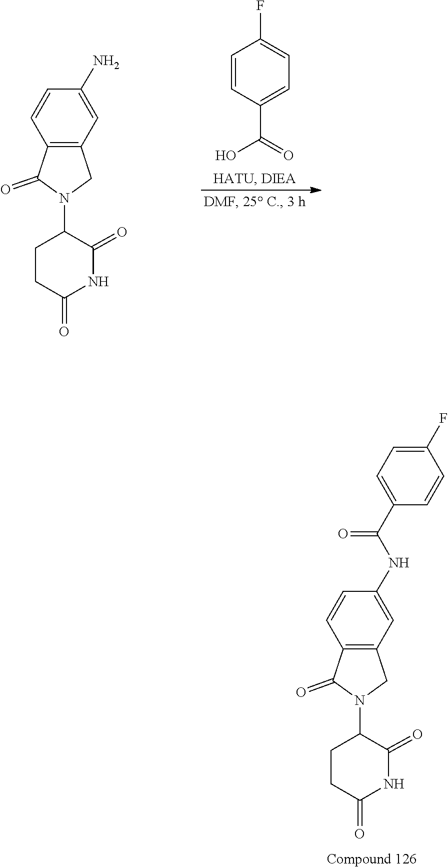 [SMILES: Nc1ccc2c(c1)CN(C1CCC(=O)NC1=O)C2=O ; O=C(O)c1ccc(F)cc1 ; O=C1CCC(N2Cc3cc(NC(=O)c4ccc(F)cc4)ccc3C2=O)C(=O)N1]

To a solution of 3-(5-amino-1-oxoisoindolin-2-yl)piperidine-2,6-dione (200 mg, 771 umol, 1.00 eq) in dimethyl formamide (5.00 mL) was added O-(7-Azabenzotriazol-1-yl)-N,N,N′,N′-tetramethyluronium Hexafluorophosphate (586 mg, 1.54 mmol, 2.00 eq) and N,N-diisopropylethylamine (398 mg, 3.09 mmol, 537 μL, 4.00 eq), then 4-fluorobenzoic acid (129 mg, 925 umol, 1.20 eq) was added. The mixture was stirred at 25° C. for 3 h. The reaction mixture was concentrated under reduced pressure to give a residue. The residue was purified by Prep-HPLC (column: Phenomenex Luna C18 100*30 mm*5 um; mobile phase: [water(formic acid)-acetonitrile]; B %: 17%-47%, 8 min) and lyophilized to afford N-(2-(2,6-dioxopiperidin-3-yl)-1-oxoisoindolin-5-yl)-4-fluorobenzamide (Compound 126, 100 mg, 262 umol, 34% yield) as a yellow solid.

¹H NMR (400 MHz, DMSO-d₆) δ=10.98 (s, 1H), 10.57 (s, 1H), 8.13 (s, 1H), 8.09-8.03 (m, 2H), 7.82 (dd, J=1.6, 8.4 Hz, 1H), 7.72 (d, J=8.4 Hz, 1H), 7.39 (t, J=9.2 Hz, 2H), 5.10 (m, 1H), 4.53-4.43 (m, 1H), 4.39-4.27 (m, 1H), 2.98-2.86 (m, 1H), 2.61 (m, 1H), 2.44-2.34 (m, 1H), 2.10-1.96 (m, 1H). MS (ESI) m/z 382.0 [M+H]⁺.

### Example 27. Synthesis of Compound 127

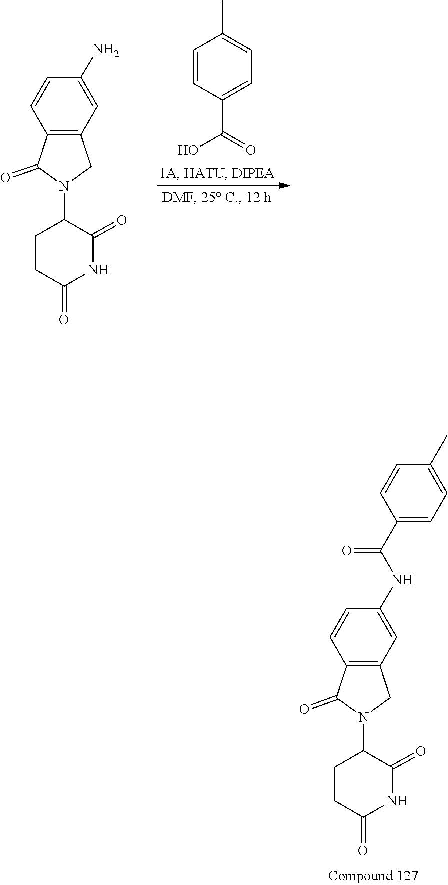

To a solution of 3-(5-amino-1-oxoisoindolin-2-yl)piperidine-2,6-dione (200 mg, 771 umol, 1.00 eq) and 4-methylbenzoic acid (126 mg, 925 umol, 1.20 eq) in dimethyformamide (2.00 mL) was added O-(7-azabenzotriazol-1-yl)-N,N,N,N-tetramethyluronium hexafluorophosphate (439 mg, 1.16 mmol, 1.50 eq) and N,N-diisopropylethylamine (299 mg, 2.31 mmol, 403 μL, 3.00 eq). The mixture was stirred at 25° C. for 12 h. The mixture was filtered. The filter cake was triturated with ethyl acetate (5 mL) at 25° C. for 15 min and then lyophilized to give N-(2-(2,6-dioxopiperidin-3-yl)-1-oxoisoindolin-5-yl)-4-methylbenzamide (Compound 127, 17.9 mg, 46.5 umol, 6% yield, 98% purity) as a yellow solid.

¹H NMR (400 MHz, DMSO-d₆) δ=11.27-10.63 (m, 1H), 10.49 (s, 1H), 8.15 (s, 1H), 7.90 (d, J=8.0 Hz, 2H), 7.84 (br d, J=8.3 Hz, 1H), 7.70 (d, J=8.4 Hz, 1H), 7.36 (d, J=7.9 Hz, 2H), 5.10 (dd, J=5.1, 13.2 Hz, 1H), 4.54-4.42 (m, 1H), 4.38-4.24 (m, 1H), 2.98-2.84 (m, 1H), 2.60 (br d, J=15.9 Hz, 1H), 2.40 (s, 4H), 2.05-1.96 (m, 1H). MS (ESI) m/z. 378.0 [M+H]⁺.

### Example 28. Synthesis of Compound 128

### Step 1.

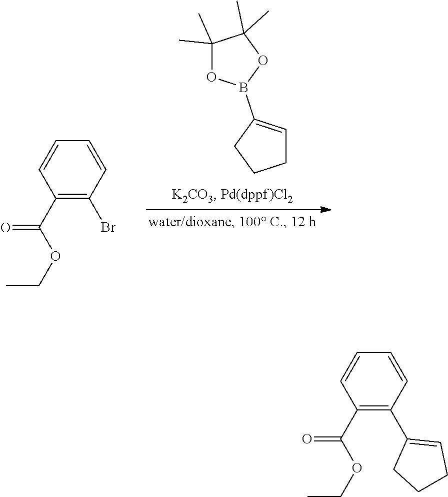 [SMILES: CCOC(=O)c1ccccc1Br ; CC1(C)OB(C2=CCCC2)OC1(C)C ; CCOC(=O)c1ccccc1C1=CCCC1]

A mixture of 2-(cyclopent-1-en-1-yl)-4,4,5,5-tetramethyl-1,3,2-dioxaborolane (2.03 g, 10.5 mmol, 1.20 eq), ethyl 2-bromobenzoate (2.00 g, 8.73 mmol, 1.00 eq), potassium carbonate (2.41 g, 17.5 mmol, 2.00 eq) and [1,1′-bis(diphenylphosphino)ferrocene]dichloropalladium(II) (0.639 g, 0.873 mmol, 0.100 eq) in water (8.00 mL) and 1,4-dioxane (32.0 mL) was stirred at 100° C. for 12 h under nitrogen atmosphere. The reaction mixture was cooled to room temperature and poured into water (30 mL). The resulting mixture was extracted with ethyl acetate (3×30 mL) and the organic layers were collected. The combined organic layers were washed with brine (3×10 mL), dried over sodium sulfate, filtered and concentrated under reduced pressure to give a residue. The residue was purified by column chromatography (SiO₂, petroleum ether/ethyl acetate=10:1) and concentrated under reduced pressure to afford ethyl 2-(cyclopent-1-en-1-yl)benzoate (2.00 g, crude) as light yellow oil.

¹H NMR (400 MHz, CDCl₃) δ=7.72-7.67 (m, 1H), 7.45-7.38 (m, 1H), 7.32-7.27 (m, 2H), 5.71 (quin, J=2.2 Hz, 1H), 4.32 (q, J=7.2 Hz, 2H), 2.70-2.58 (m, 2H), 2.51 (qt, J=2.4, 7.4 Hz, 2H), 2.03 (quin, J=7.4 Hz, 2H), 1.35 (t, J=7.2 Hz, 3H).

### Step 2.

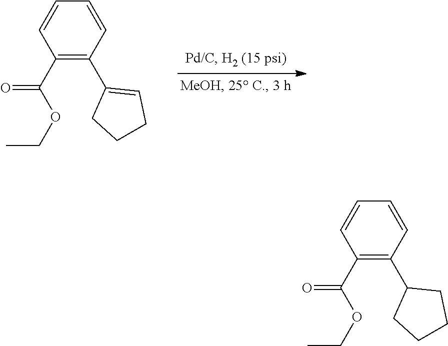 [SMILES: CCOC(=O)c1ccccc1C1=CCCC1 ; CCOC(=O)c1ccccc1C1CCCC1]

To a solution of ethyl 2-(cyclopent-1-en-1-yl)benzoate (2.00 g, 9.25 mmol, 1.00 eq) in methanol (50.0 mL) was added Palladium on carbon (0.230 g, 10% purity) under nitrogen atmosphere. The suspension was degassed and purged with hydrogen for 3 times. The mixture was stirred under hydrogen (15 Psi) at 25° C. for 3 h. The reaction mixture was filtered and the filtrate was collected and concentrated under reduced pressure to afford ethyl 2-cyclopentylbenzoate (2.00 g, crude) as light yellow oil.

¹H NMR (400 MHz, CDCl₃) δ 7.70 (d, J=7.4 Hz, 1H), 7.49-7.36 (m, 2H), 7.25-7.16 (m, 1H), 4.37 (q, J=7.1 Hz, 2H), 3.72 (td, J=8.5, 16.8 Hz, 1H), 2.10 (br d, J=5.4 Hz, 2H), 1.82 (br d, J=6.2 Hz, 2H), 1.76-1.67 (m, 2H), 1.65-1.57 (m, 2H), 1.40 (t, J=7.2 Hz, 3H).

### Step 3.

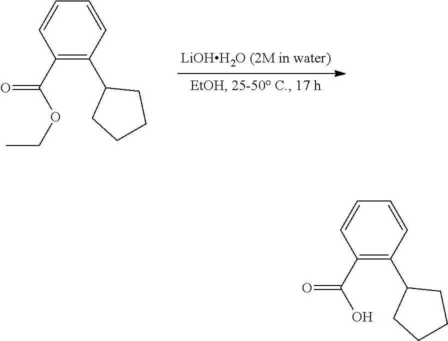 [SMILES: CCOC(=O)c1ccccc1C1CCCC1 ; O=C(O)c1ccccc1C1CCCC1]

To a solution of ethyl 2-cyclopentylbenzoate (2.00 g, 9.16 mmol, 1.00 eq) in ethanol (10.0 mL) was added lithium hydroxide hydrate (2.00 M, 10.0 mL, 2.06 eq, water solution). The reaction mixture was stirred at 25° C. for 12 h. Then the reaction mixture was stirred at 50° C. for another 5 h. The reaction mixture was concentrated under reduced pressure to remove the organic layer. The pH of aqueous was adjusted to 7 with hydrochloric acid (1M). The mixture was filtered and the filter cake was dried under reduced pressure to afford 2-cyclopentylbenzoic acid (1.50 g, crude) as an off-white solid.

¹H NMR (400 MHz, DMSO-d₆) δ 13.60-12.07 (m, 1H), 7.61 (d, J=7.6 Hz, 1H), 7.49-7.37 (m, 2H), 7.29-7.15 (m, 1H), 3.75-3.63 (m, 1H), 1.98 (s, 2H), 1.83-1.69 (m, 2H), 1.68-1.43 (m, 4H).

### Step 4.

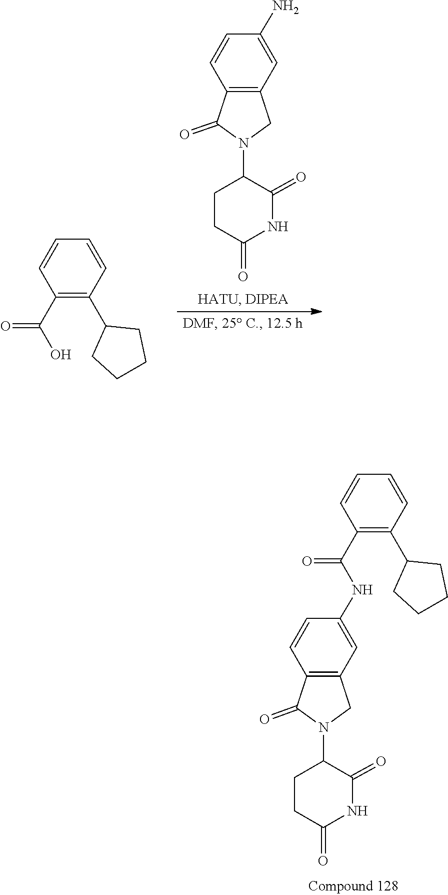 [SMILES: O=C(O)c1ccccc1C1CCCC1 ; Nc1ccc2c(c1)CN(C1CCC(=O)NC1=O)C2=O ; O=C1CCC(N2Cc3cc(NC(=O)c4ccccc4C4CCCC4)ccc3C2=O)C(=O)N1]

A mixture of 3-(5-amino-1-oxo-isoindolin-2-yl)piperidine-2,6-dione (150 mg, 579 umol, 1.10 eq), N,N-diisopropylethylamine (204 mg, 1.58 mmol, 3.00 eq) and O-(7-Azabenzotriazol-1-yl)-N,N,N′,N′-tetramethyluronium hexafluorophosphate (400 mg, 1.05 mmol, 2.00 eq) in dimethyl formamide (3.00 mL) was stirred at 25° C. for 0.5 h. Then, 2-cyclopentylbenzoic acid (100 mg, 526 umol, 1.00 eq) was added and the resulting mixture was stirred at 25° C. for 12 h. The reaction mixture was poured into water (30 mL). The resulting mixture was extracted with ethyl acetate (3×30 mL) and the organic layers were collected. The combined organic layers were washed with brine (3×10 mL), dried over sodium sulfate, filtered and concentrated under reduced pressure to give a residue. The residue was purified by Prep-HPLC (column: Phenomenex Luna C18 100×30 mm×5 μm; mobile phase: [water (formic acid)-acetonitrile]; B %: 30%-60%, 8 min) and lyophilized to afford 2-cyclopentyl-N-(2-(2,6-dioxopiperidin-3-yl)-1-oxoisoindolin-5-yl)benzamide formate (Compound 128, 36.5 mg, 830 umol, 16% yield, 98% purity) as a white solid.

¹H NMR (400 MHz, DMSO-d₆) δ 11.08-10.83 (m, 1H), 10.72 (s, 1H), 8.41 (s, 1H), 8.16 (s, 1H), 7.80-7.65 (m, 2H), 7.54-7.37 (m, 3H), 7.35-7.24 (m, 1H), 5.10 (m, 1H), 4.51-4.27 (m, 2H), 3.27-3.20 (m, 1H), 3.00-2.84 (m, 1H), 2.58 (s, 1H), 2.44-2.33 (m, 1H), 2.07-1.93 (m, 3H), 1.76 (s, 2H), 1.64-1.51 (m, 4H). MS (ESI) m/z 432.1 [M+H]⁺.

### Example 29. Synthesis of Compound 129

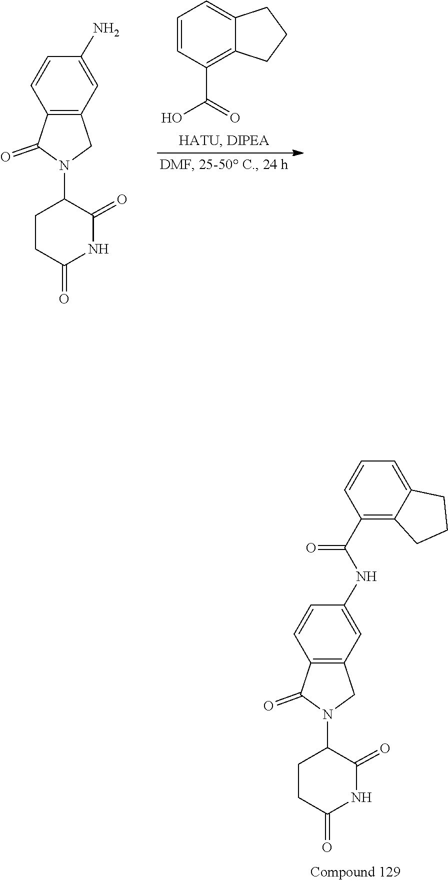 [SMILES: Nc1ccc2c(c1)CN(C1CCC(=O)NC1=O)C2=O ; O=C(O)c1cccc2c1CCC2 ; O=C1CCC(N2Cc3cc(NC(=O)c4cccc5c4CCC5)ccc3C2=O)C(=O)N1]

To a solution of 3-(5-amino-1-oxo-isoindolin-2-yl)piperidine-2,6-dione (100 mg, 385 umol, 1.00 eq), 2,3-dihydro-1H-indene-4-carboxylic acid (75.0 mg, 462 umol, 1.20 eq) in dimethyformamide (2.00 mL) was added O-(7-azabenzotriazol-1-yl)-N,N,N,N-tetramethyluronium hexafluorophosphate (219 mg, 578 umol, 1.50 eq) and N,N-diisopropylethylamine (149 mg, 1.16 mmol, 201 μL, 3.00 eq). The mixture was stirred at 25° C. for 12 h. Then the mixture was stirred at 50° C. for 12 h. The solution was adjusted pH to 6 by formic acid (0.1 mL) and filtered. The filtrate was purified by Prep-HPLC (column: Phenomenex Luna C18 100*30 mm*5 um; mobile phase: [water(formic acid)-acetonitrile]; B %: 25%-55%, 8 min) and lyophilized to afford N-(2-(2,6-dioxopiperidin-3-yl)-1-oxoisoindolin-5-yl)-2,3-dihydro-1H-indene-4-carboxamide (Compound 129, 27.34 mg, 67.1 umol, 17% yield, 99% purity) as a white solid.

¹H NMR (400 MHz, DMSO-d₆) δ=10.98 (s, 1H), 10.49 (s, 1H), 8.13 (s, 1H), 7.80-7.75 (m, 1H), 7.70 (s, 1H), 7.50 (d, J=7.5 Hz, 1H), 7.43 (d, J=7.4 Hz, 1H), 7.32-7.26 (m, 1H), 5.10 (m, 1H), 4.50-4.43 (m, 1H), 4.36-4.28 (m, 1H), 3.08 (m, 2H), 2.96-2.87 (m, 3H), 2.61 (m, 1H), 2.35 (br s, 1H), 2.08-1.97 (m, 3H). MS (ESI) m/z. 403.9 [M+H]⁺.

### Example 30. Synthesis of Compound 130

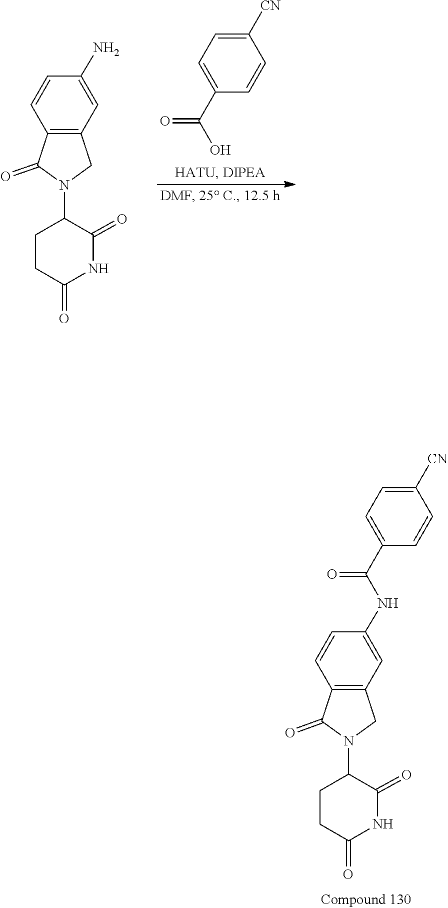 [SMILES: Nc1ccc2c(c1)CN(C1CCC(=O)NC1=O)C2=O ; N#Cc1ccc(C(=O)O)cc1 ; N#Cc1ccc(C(=O)Nc2ccc3c(c2)CN(C2CCC(=O)NC2=O)C3=O)cc1]

A mixture of 4-cyanobenzoic acid (71 mg, 482 umol, 1.00 eq), N,N-diisopropylethylamine (187 mg, 1.45 umol, 3.00 eq) and O-(7-Azabenzotriazol-1-yl)-N,N,N′,N′-tetramethyluronium hexafluorophosphate (367 mg, 964 umol, 2.00 eq) in dimethylformamide (3.00 mL) was stirred at 25° C. for 0.5 h. Then 3-(5-amino-1-oxo-isoindolin-2-yl)piperidine-2,6-dione (150 mg, 579 umol, 1.20 eq) was added and the resulting mixture was stirred at 25° C. for 12 h. The mixture was filtered and the filtrate was concentrated under reduced pressure to give a residue. The residue was purified by Prep-HPLC (column: Phenomenex Luna C18 150×30 mm×5 μm; mobile phase: [water (formic acid)-acetonitrile]; B %: 16%-46%, 9 min) and lyophilized to afford 4-cyano-N-(2-(2,6-dioxopiperidin-3-yl)-1-oxoisoindolin-5-yl)benzamide (18.7 mg, 48 umol, 10% yield, 99% purity) as a white solid.

¹H NMR (400 MHz, DMSO-d₆) δ=10.99 (s, 1H), 10.78 (s, 1H), 8.13 (d, J=8.2 Hz, 3H), 8.08-8.03 (m, 2H), 7.83 (dd, J=1.4, 8.4 Hz, 1H), 7.73 (d, J=8.4 Hz, 1H), 5.11 (m, 1H), 4.53-4.29 (m, 2H), 2.98-2.86 (m, 1H), 2.63 (s, 1H), 2.41 (m, 1H), 2.05-1.97 (m, 1H). MS (ESI) m/z. 389.0 [M+H]⁺.

### Example 31. Synthesis of Compound 131

### Step 1.

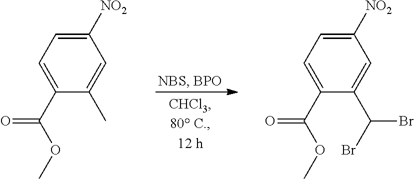

To a solution of methyl 2-methyl-4-nitrobenzoate (50.0 g, 256 mmol, 1.00 eq) in trichloromethane (500 mL) was added N-Bromosuccinimide (91.1 g, 512 mmol, 2.00 eq), benzoyl peroxide (6.21 g, 25.6 mmol, 0.100 eq). The mixture was stirred at 80° C. for 12 h. The mixture was filtered and the filtrate was concentrated under reduced pressure to give a residue. The residue was diluted with saturated sodium carbonate (200 mL), extracted with ethyl acetate (3×200 mL). The combined organic layers were dried over anhydrous sodium sulfate, filtered and concentrated under reduced pressure to afford methyl 2-(dibromomethyl)-4-nitrobenzoate (70.0 g, crude) as yellow oil.

### Step 2.

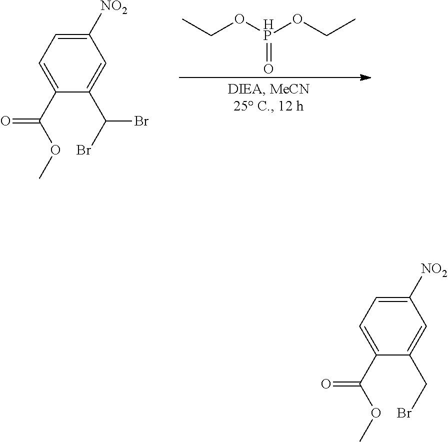

To a solution of methyl 2-(dibromomethyl)-4-nitrobenzoate (70.0 g, 198 mmol, 1.00 eq), N,N-diisopropylethylamine (12.8 g, 99.1 mmol, 17.2 mL, 0.500 eq) in acetonitrile (500 mL) was added diethyl phosphonate (10.9 g, 79.3 mmol, 10.2 mL, 0.400 eq). The mixture was stirred at 25° C. for 12 h. The mixture was concentrated under reduced pressure to give a residue. The mixture was diluted with water (300 mL), extracted with ethyl acetate (2×300 mL). The combined organic layers were dried over anhydrous sodium sulfate, filtered and concentrated under reduced pressure to afford methyl 2-(bromomethyl)-4-nitrobenzoate (70.0 g, crude) as a yellow solid.

¹H NMR (400 MHz, CDCl₃) δ=8.34 (d, J=2.1 Hz, 1H), 8.23-8.18 (m, 1H), 8.14-8.10 (m, 1H), 4.97 (s, 2H), 4.01 (s, 3H).

### Step 3.

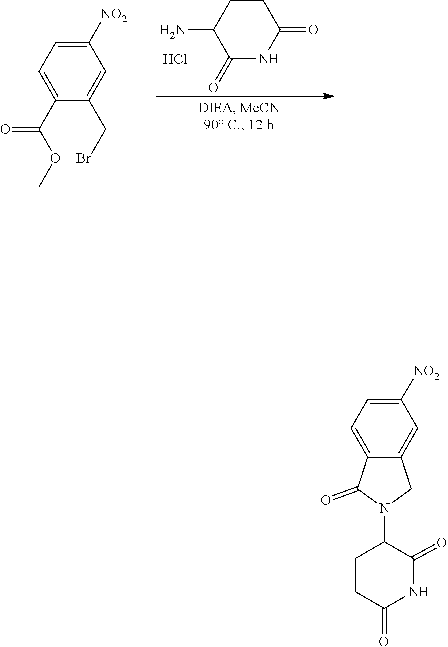 [SMILES: COC(=O)c1ccc([N+](=O)[O-])cc1CBr ; Cl ; NC1CCC(=O)NC1=O ; O=C1CCC(N2Cc3cc([N+](=O)[O-])ccc3C2=O)C(=O)N1]

To a solution of methyl 2-(bromomethyl)-4-nitro-benzoate (10.0 g, 36.4 mmol, 1.00 eq), 3-aminopiperidine-2,6-dione; hydrochloride (4.20 g, 25.5 mmol, 0.700 eq) in acetonitrile (150 mL) was added N,N-diisopropylethylamine (14.1 g, 109 mmol, 19.0 mL, 3.00 eq). The mixture was stirred at 90° C. for 12 h. The mixture was concentrated under reduced pressure to give a residue. The residue was triturated with hydrochloric acid (1M, 100 mL) at 20° C. for 0.5 h and filtered. The filter cake was triturated with ethanol (100 mL) at 20° C. for 0.5 h and filtered again. The filter cake was dried under reduced pressure to afford 3-(5-nitro-1-oxoisoindolin-2-yl)piperidine-2,6-dione (5.40 g, 18.6 mmol, 51% yield) as a brown solid.

¹H NMR (400 MHz, DMSO-d₆) δ=11.04 (s, 1H), 8.52 (d, J=1.6 Hz, 1H), 8.35 (dd, J=2.1, 8.3 Hz, 1H), 7.97 (d, J=8.4 Hz, 1H), 5.17 (dd, J=5.1, 13.3 Hz, 1H), 4.65-4.56 (m, 1H), 4.53-4.45 (m, 1H), 2.98-2.86 (m, 1H), 2.62 (br d, J=16.1 Hz, 1H), 2.47-2.37 (m, 1H), 2.10-2.01 (m, 1H).

### Step 4.

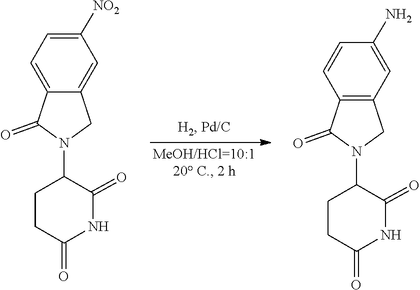 [SMILES: O=C1CCC(N2Cc3cc([N+](=O)[O-])ccc3C2=O)C(=O)N1 ; Nc1ccc2c(c1)CN(C1CCC(=O)NC1=O)C2=O]

To a solution of 3-(5-nitro-1-oxoisoindolin-2-yl)piperidine-2,6-dione (5.40 g, 18.6 mmol, 1.00 eq) in methanol (60.0 mL) was added hydrochloric acid (12 M, 5.00 mL), Palladium on carbon (1.00 g, 10% purity). The mixture was stirred at 20° C. for 2 h under hydrogen atmosphere (15 PSI). The mixture was diluted with hydrochloric acid (1M, 60 mL), filtered and the filtrate was concentrated to remove solvent (methanol) and lyophilized to give a crude product. The crude product was triturated with ethanol (50 mL) at 20° C. for 0.5 h and filtered. The filter cake was dried under reduced pressure to afford 3-(5-amino-1-oxoisoindolin-2-yl)piperidine-2,6-dione (3.20 g, 11.4 mmol, 61% yield) as a gray solid.

¹H NMR (400 MHz, DMSO-d₆) δ=10.96 (s, 1H), 7.61 (d, J=8.1 Hz, 1H), 7.18 (s, 1H), 7.12 (br d, J=8.1 Hz, 1H), 5.06 (dd, J=5.1, 13.3 Hz, 1H), 4.43-4.36 (m, 1H), 4.29-4.23 (m, 1H), 2.95-2.85 (m, 1H), 2.59 (br d, J=18.0 Hz, 1H), 2.40-2.31 (m, 1H), 2.02-1.95 (m, 1H).

### Step 5.

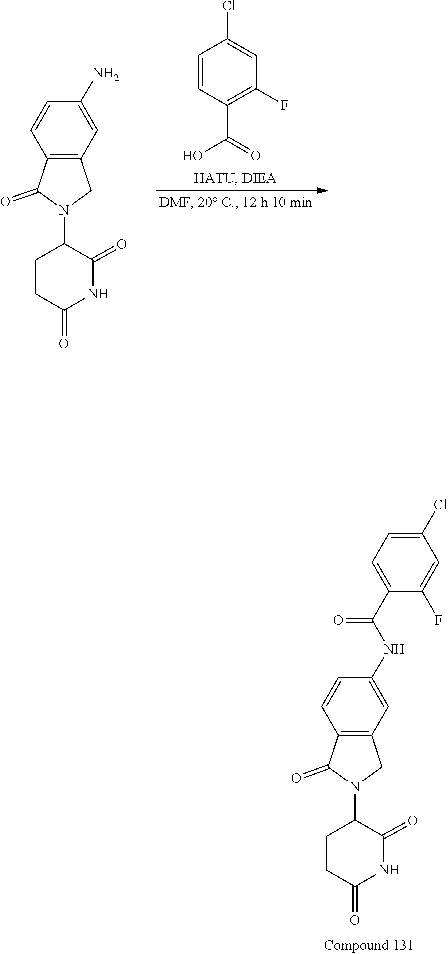 [SMILES: Nc1ccc2c(c1)CN(C1CCC(=O)NC1=O)C2=O ; O=C(O)c1ccc(Cl)cc1F ; O=C1CCC(N2Cc3cc(NC(=O)c4ccc(Cl)cc4F)ccc3C2=O)C(=O)N1]

To a solution of 4-chloro-2-fluorobenzoic acid (80.8 mg, 463 umol, 1.20 eq) and N, N-diisopropylethylamine (150 mg, 1.16 mmol, 202 μL, 3.00 eq) in N, N-dimethyl formamide (2.00 mL) was added O-(7-Azabenzotriazol-1-yl)-N, N, N, N-tetramethyl uronium hexafluorophosphate (220 mg, 579 umol, 1.50 eq). The mixture was stirred at 20° C. for 10 min. Then 3-(5-amino-1-oxoisoindolin-2-yl) piperidine-2,6-dione (100 mg, 386 umol, 1.00 eq) was added. The mixture was stirred at 20° C. for 12 h. The reaction mixture was diluted with water (20 mL), extracted with ethyl acetate (3×15 mL). The combined organic layers were dried over with anhydrous sodium sulfate, filtered and concentrated under reduced pressure to give a residue. The residue purified by Prep-HPLC (column: Waters xbridge 150*25 mm 10 um; mobile phase: [water (ammonium bicarbonate)-acetonitrile]; B %: 20%-50%, 8 min) and lyophilized to afford 4-chloro-N-(2-(2,6-dioxopiperidin-3-yl)-1-oxoisoindolin-5-yl)-2-fluorobenzamide (Compound 131, 35.66 mg, 91.8 umol, 24% yield, 97% purity) as a yellow solid.

¹H NMR (400 MHz, DMSO-d₆) δ=10.97 (s, 1H), 10.80 (s, 1H), 8.07 (s, 1H), 7.74 (s, 1H), 7.72 (s, 2H), 7.66 (dd, J=1.9, 10.0 Hz, 1H), 7.46 (dd, J=1.8, 8.3 Hz, 1H), 5.10 (m, 1H), 4.51-4.43 (m, 1H), 4.38-4.29 (m, 1H), 2.99-2.84 (m, 1H), 2.70-2.60 (m, 1H), 2.39-2.30 (m, 1H), 2.06-1.96 (m, 1H). MS (ESI) m/z 416.0 [M+H]⁺.

### Example 32. Synthesis of Compound 132

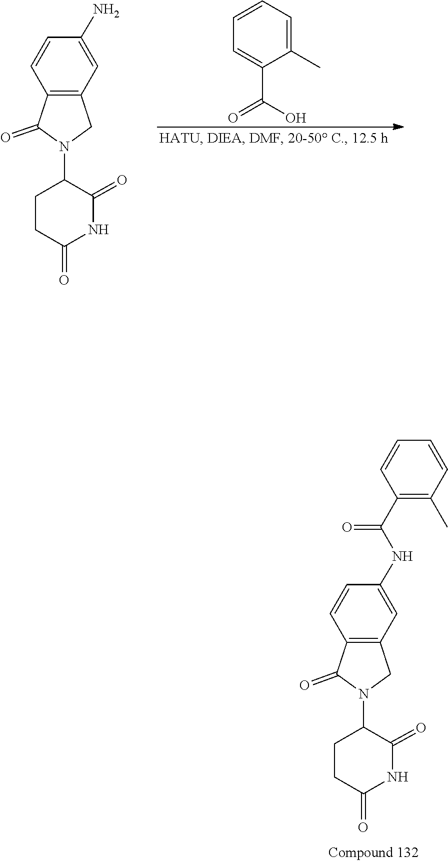

To a solution of 2-methylbenzoic acid (158 mg, 116 umol, 3.00 eq) in dimethyl formamide (3.00 mL) was added N,N-diisopropylethylamine (150 mg, 116 umol, 3.00 eq) and O-(7-azabenzotriazol-1-yl)-N,N,N,N-tetramethyluroniumhexafluorophosphate (880 mg, 231 umol, 6.00 eq) at 20° C. The mixture was stirred at 20° C. for 30 min, then 3-(5-amino-1-oxo-isoindolin-2-yl)piperidine-2,6-dione (100 mg, 386 umol, 1.00 eq) was added. The resulting mixture was stirred at 50° C. for 12 h. The reaction mixture was quenched by addition formic acid (5.00 mL) and filtered to give a filtrate. The filtrate was purified by Prep-HPLC column: (Phenomenex luna C18 150*25 mm*10 um; mobile phase: [water(formic acid)-acetonitrile]; B %: 18%-48%, 9 min) and lyophilized to afford N-(2-(2,6-dioxo-3-piperidyl)-1-oxo-isoindolin-5-yl)-2-methyl-benzamide (Compound 132, 46.2 mg, 121 umol, 31% yield, 99.0% purity) as a white solid.

¹H NMR (400 MHz, DMSO-d₆) δ=11.0 (s, 1H), 10.7 (s, 1H), 8.15 (s, 1H), 7.78-7.73 (m, 1H), 7.72-7.68 (m, 1H), 7.50 (d, J=7.6 Hz, 1H), 7.45-7.39 (m, 1H), 7.36-7.30 (m, 2H), 5.11 (m, 1H), 4.51-4.44 (m, 1H), 4.36-4.30 (m, 1H), 2.98-2.86 (m, 1H), 2.61 (m, 1H), 2.42-2.38 (m, 4H), 2.06-1.97 (m, 1H). MS (ESI) m/z 378.1 [M+H]⁺.

### Example 33. Synthesis of Compound 133

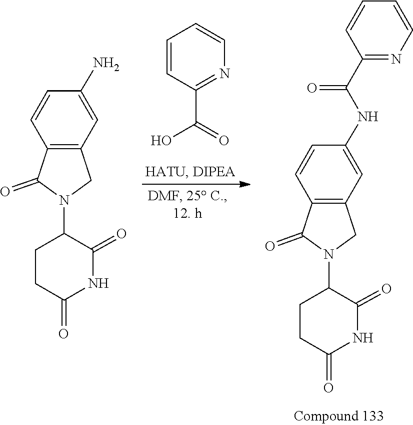 [SMILES: Nc1ccc2c(c1)CN(C1CCC(=O)NC1=O)C2=O ; O=C(O)c1ccccn1 ; O=C1CCC(N2Cc3cc(NC(=O)c4ccccn4)ccc3C2=O)C(=O)N1]

To a solution of 3-(5-amino-1-oxoisoindolin-2-yl)piperidine-2,6-dione (100 mg, 385 umol, 1.00 eq) in dimethyformamide (2.00 mL) was added O-(7-azabenzotriazol-1-yl)-N,N,N,N-tetramethyluronium hexafluorophosphate (219 mg, 578 umol, 1.50 eq), N,N-diisopropylethylamine (149 mg, 1.16 mmol, 201 μL, 3.00 eq) and picolinic acid (56.9 mg, 462 umol, 1.20 eq) in portions. The mixture was stirred at 25° C. for 12 h. Then the pH was adjusted to 6 with formic acid (0.1 mL) and filtered. The filtrate was purified by Prep-HPLC (column: Phenomenex Luna C18 100*30 mm*5 um; mobile phase: [water(formic acid)-acetonitrile]; B %: 14%-44%, 8 min) and lyophilized to afford N-(2-(2,6-dioxopiperidin-3-yl)-1-oxoisoindolin-5-yl) (Compound 133, 9.62 mg, 24.8 mol, 6% yield, 94% purity) as a gray solid.

¹H NMR (400 MHz, DMSO-d₆) δ=10.95 (s, 1H), 11.03-10.91 (m, 1H), 8.77 (d, J=4.4 Hz, 1H), 8.27 (s, 1H), 8.19 (d, J=7.8 Hz, 1H), 8.09 (dt, J=1.6, 7.7 Hz, 1H), 8.00 (dd, J=1.7, 8.3 Hz, 1H), 7.76-7.62 (m, 2H), 5.10 (m, 1H), 4.55-4.41 (m, 1H), 4.40-4.27 (m, 1H), 2.99-2.85 (m, 1H), 2.61 (m, 1H), 2.46-2.35 (m, 1H), 2.06-1.96 (m, 1H). MS (ESI) m/z. 365.0 [M+H]⁺.

### Example 34. Synthesis of Compound 134

### Step 1.

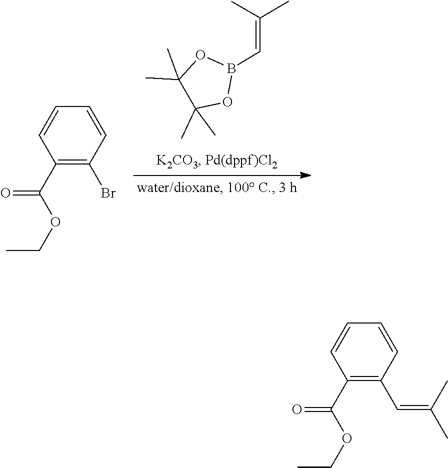

A mixture of ethyl 2-bromobenzoate (2.00 g, 8.73 mmol, 1.00 eq), 4,4,5,5-tetramethyl-2-(2-methylprop-1-en-1-yl)-1,3,2-dioxaborolane (2.38 g, 13.1 mmol, 1.50 eq), potassium carbonate (2.41 g, 17.5 mmol, 2.00 eq) and [1,1′-bis(diphenylphosphino)ferrocene]dichloropalladium(II) (0.639 g, 0.873 mmol, 0.100 eq) in water (8.00 mL) and 1,4-dioxane (32.0 mL) was stirred at 100° C. for 3 h under nitrogen atmosphere. The reaction mixture was cooled to room temperature and poured into water (30 mL). The resulting mixture was extracted with ethyl acetate (3×30 mL) and the organic layers were collected. The combined organic layers were washed with brine (3×10 mL), dried over sodium sulfate, filtered and concentrated under reduced pressure to give a residue. The residue was purified by column chromatography (SiO₂, petroleum ether/ethyl acetate=10:1) and concentrated under reduced pressure to afford ethyl 2-(2-methylprop-1-en-1-yl)benzoate (2.30 g, crude) as light yellow oil.

¹H NMR (400 MHz, CDCl₃) δ=7.90 (dd, J=1.2, 7.8 Hz, 1H), 7.48-7.41 (m, 1H), 7.27 (s, 2H), 6.65 (s, 1H), 4.34 (q, J=7.2 Hz, 2H), 1.93 (d, J=1.4 Hz, 3H), 1.71 (d, J=1.0 Hz, 3H), 1.38 (t, J=7.2 Hz, 3H).

### Step 2.

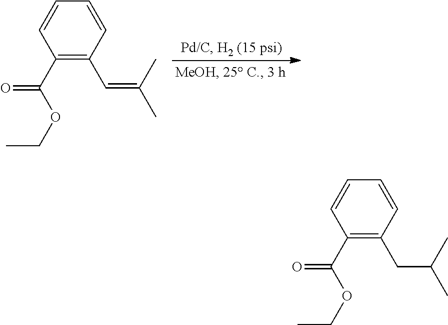

To a solution of ethyl 2-(2-methylprop-1-en-1-yl)benzoate (2.30 g, 11.3 mmol, 1.00 eq) in methanol (50.0 mL) was added Palladium on carbon (0.230 g, 10% purity) under nitrogen atmosphere. The suspension was degassed and purged with hydrogen for 3 times. The mixture was stirred under hydrogen (15 Psi) at 25° C. for 3 h. The reaction mixture was filtered and the filtrate was concentrated under reduced pressure to afford ethyl 2-isobutylbenzoate (2.00 g, crude) as light yellow oil.

¹H NMR (400 MHz, CDCl₃) δ=7.85 (d, J=7.8 Hz, 1H), 7.43-7.36 (m, 1H), 7.27-7.18 (m, 2H), 4.40-4.33 (m, 2H), 2.86 (d, J=7.0 Hz, 2H), 1.87 (quind, J=6.4, 13.2 Hz, 1H), 1.45-1.35 (m, 3H), 0.92 (d, J=0.8 Hz, 3H), 0.90 (s, 3H).

### Step 3.

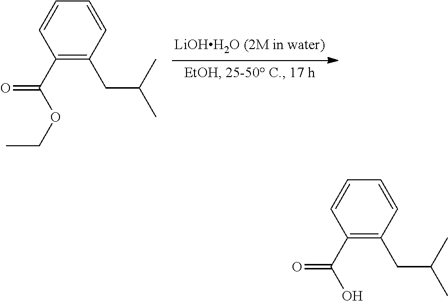

To a solution of ethyl 2-isobutylbenzoate (2.00 g, 9.70 mmol, 1.00 eq) in ethanol (10.0 mL) was added lithium hydroxide hydrate (2.00 M, 10 mL, 2.06 eq, water solution). The reaction mixture was stirred at 25° C. for 12 h. Then the reaction mixture was stirred at 50° C. for another 5 h. The reaction mixture was concentrated under reduced pressure to remove the organic layer. The pH of aqueous phase was adjusted to 7 with hydrochloric acid (1 M). The mixture was filtered and the filter cake was concentrated under reduced pressure to afford 2-isobutylbenzoic acid (1.00 g, crude) as an off-white solid.

¹H NMR (400 MHz, DMSO-d₆) δ=12.77 (s, 1H), 7.76 (d, J=7.8 Hz, 1H), 7.48-7.38 (m, 1H), 7.32-7.18 (m, 2H), 2.81 (d, J=7.2 Hz, 2H), 1.79 (td, J=6.8, 13.4 Hz, 1H), 0.84 (s, 3H), 0.82 (s, 3H).

### Step 4.

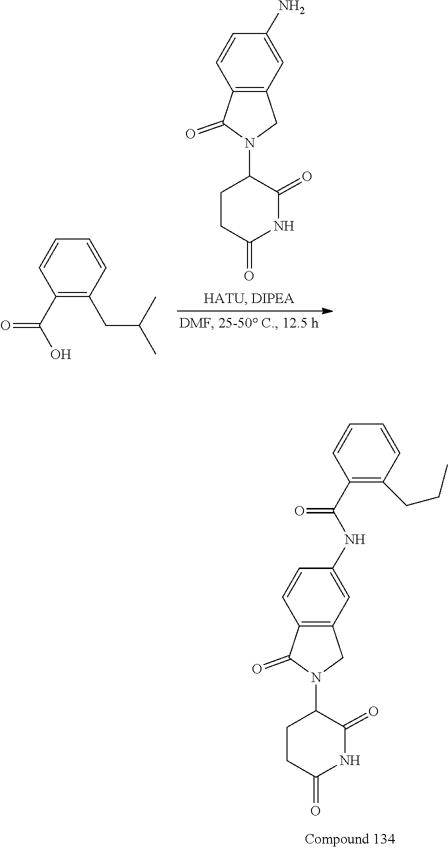 [SMILES: CC(C)Cc1ccccc1C(=O)O ; Nc1ccc2c(c1)CN(C1CCC(=O)NC1=O)C2=O ; CCCc1ccccc1C(=O)Nc1ccc2c(c1)CN(C1CCC(=O)NC1=O)C2=O]

A mixture of 3-(5-amino-1-oxoisoindolin-2-yl)piperidine-2,6-dione (150 mg, 579 umol, 1.10 eq), N,N-diisopropylethylamine (204 mg, 1.58 mmol, 3.00 eq) and O-(7-Azabenzotriazol-1-yl)-N,N,N′,N′-tetramethyluronium hexafluorophosphate (400 mg, 1.05 mmol, 2.00 eq) in dimethyl formamide (3.00 mL) was stirred at 25° C. for 0.5 h. Then 2-isobutylbenzoic acid (93.7 mg, 526 umol, 1.00 eq) was added and the resulting mixture was stirred at 50° C. for 12 h. The reaction mixture was cooled to room temperature and poured into water (30 mL). The resulting mixture was extracted with ethyl acetate (3×30 mL) and the organic layers were collected. The combined organic layers were washed with brine (3×10 mL), dried over sodium sulfate, filtered and concentrated in vacuo. The residue was purified by prep-HPLC (column: Phenomenex Luna C18 100×30 mm×5 um; mobile phase: [water (formic acid)-acetonitrile]; B %: 30%-60%, 8 min) and lyophilized to afford N-(2-(2,6-dioxopiperidin-3-yl)-1-oxoisoindolin-5-yl)-2-isobutylbenzamide (Compound 134, 72.3 mg, 171 umol, 32% yield, 99% purity) as a white solid.

¹H NMR (400 MHz, DMSO-d₆) δ=10.98 (s, 1H), 10.68 (s, 1H), 8.13 (s, 1H), 7.76-7.65 (m, 2H), 7.50-7.40 (m, 2H), 7.37-7.27 (m, 2H), 5.10 (m, 1H), 4.52-4.28 (m, 2H), 2.99-2.84 (m, 1H), 2.66 (m, 2H), 2.60 (m, 1H), 2.45-2.32 (m, 1H), 2.05-1.95 (m, 1H), 1.84 (m, 1H), 0.83 (s, 3H), 0.81 (s, 3H). MS (ESI) m/z 420.1 [M+H]⁺.

### Example 35. Synthesis of Compound 135

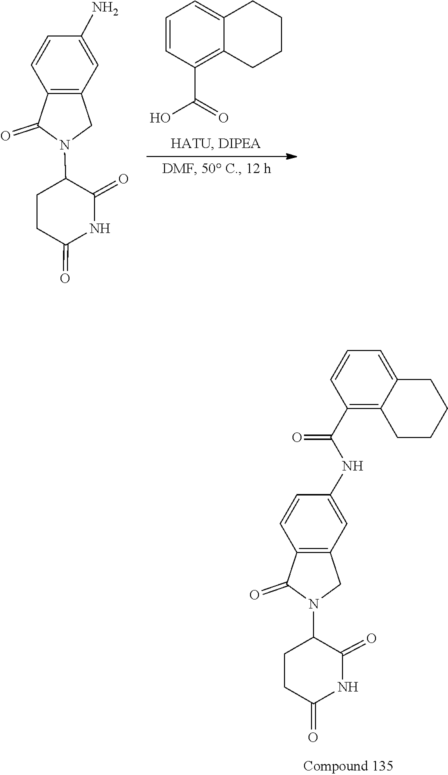 [SMILES: Nc1ccc2c(c1)CN(C1CCC(=O)NC1=O)C2=O ; O=C(O)c1cccc2c1CCCC2 ; O=C1CCC(N2Cc3cc(NC(=O)c4cccc5c4CCCC5)ccc3C2=O)C(=O)N1]

To a solution of 3-(5-amino-1-oxo-isoindolin-2-yl)piperidine-2,6-dione (100 mg, 385 umol, 1.00 eq) and 5,6,7,8-tetrahydronaphthalene-1-carboxylic acid (81.5 mg, 462.8 umol, 1.20 eq) in dimethyformamide (1.00 mL) was added O-(7-azabenzotriazol-1-yl)-N,N,N,N-tetramethyluronium hexafluorophosphate (219 mg, 578 umol, 1.50 eq) and N,N-diisopropylethylamine (149 mg, 1.16 mmol, 201 μL, 3.00 eq). The mixture was stirred at 50° C. for 12 h. The reaction mixture was filtered. The filtrate was purified by Prep-HPLC phase-HPLC(column: Phenomenex luna C18 150*25 mm*10 um; mobile phase: [water(hydrochloric acid)-acetonitrile]; B %: 28%-58%, 10 min) and lyophilized to afford N-(2-(2,6-dioxopiperidin-3-yl)-1-oxoisoindolin-5-yl)-5,6,7,8-tetrahydronaphthalene-1-carboxamide (Compound 135, 49.94 mg, 117 umol, 30% yield, 98% purity) as an off-white solid.

¹H NMR (400 MHz, DMSO-d₆) δ=10.98 (s, 1H), 10.61 (s, 1H), 8.19-8.06 (m, 1H), 7.83-7.53 (m, 2H), 7.30-7.15 (m, 3H), 5.10 (m, 1H), 4.54-4.41 (m, 1H), 4.37-4.22 (m, 1H), 2.97-2.84 (m, 1H), 2.79 (br s, 4H), 2.58 (br s, 1H), 2.45-2.34 (m, 1H), 2.10-1.94 (m, 1H), 1.74 (br s, 4H). MS (ESI) m/z 418.1 [M+H]⁺.

### Example 36. Synthesis of Compound 136

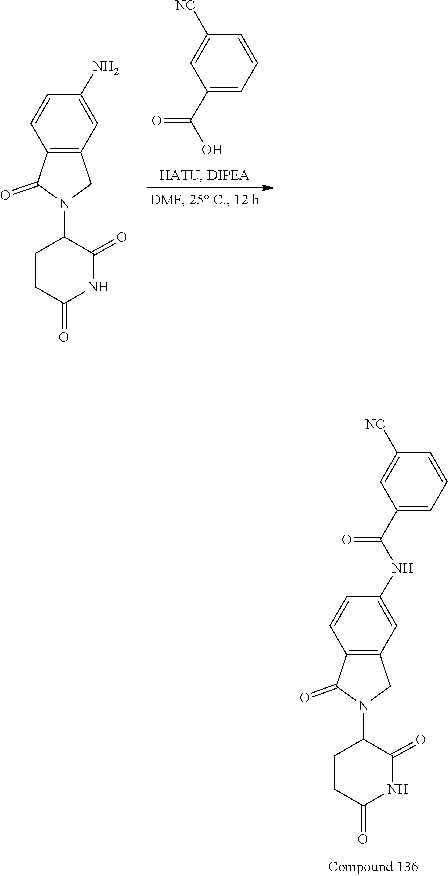 [SMILES: Nc1ccc2c(c1)CN(C1CCC(=O)NC1=O)C2=O ; N#Cc1cccc(C(=O)O)c1 ; N#Cc1cccc(C(=O)Nc2ccc3c(c2)CN(C2CCC(=O)NC2=O)C3=O)c1]

To a solution of 3-(5-amino-1-oxoisoindolin-2-yl)piperidine-2,6-dione (100 mg, 386 umol, 1.00 eq) and 3-cyanobenzoic acid (68.1 mg, 463 umol, 1.20 eq) in dimethyformamide (2.00 mL) was added O-(7-azabenzotriazol-1-yl)-N,N,N′,N′-tetramethyluronium hexafluorophosphate (220 mg, 579 umol, 1.50 eq) and N,N-diisopropylethylamine (149 mg, 1.16 mmol, 202 μL, 3.00 eq). The mixture was stirred at 25° C. for 12 h. The mixture was adjusted pH<7 with formic acid (0.1 mL) and filtered. The filtrate was purified by prep-HPLC (column: Phenomenex luna C18 150*25 mm*10 um; mobile phase: [water(FA)-ACN]; B %: 15%-45%, 9 min) and lyophilized to afford 3-cyano-N-(2-(2,6-dioxopiperidin-3-yl)-1-oxoisoindolin-5-yl) benzamide (Compound 136, 31 mg, 79.0 umol, 20% yield, 99% purity) as a brown solid.

¹H NMR (400 MHz, DMSO-d₆) δ=10.98 (s, 1H), 10.71 (s, 1H), 8.43 (s, 1H), 8.27 (br d, J=8.0 Hz, 1H), 8.17-8.01 (m, 2H), 7.85-7.67 (m, 3H), 5.11 (dd, J=5.1, 13.3 Hz, 1H), 4.54-4.32 (m, 2H), 3.00-2.82 (m, 1H), 2.61 (br d, J=16.5 Hz, 1H), 2.44-2.31 (m, 1H), 2.07-1.97 (m, 1H). MS (ESI) m/z. 389.0 [M+H]⁺.

### Example 37. Synthesis of Compound 137

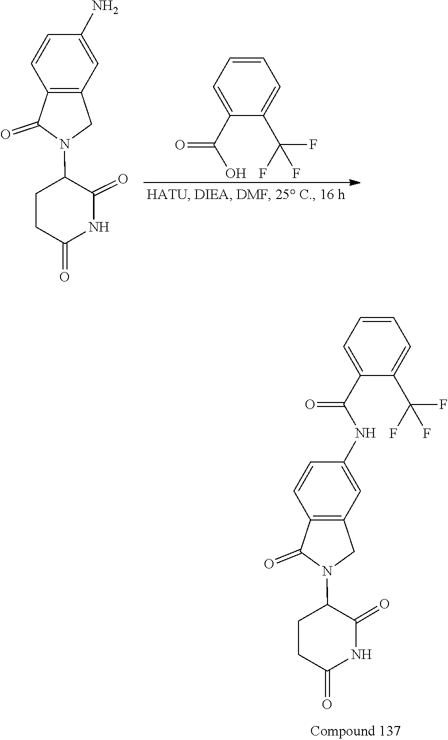 [SMILES: Nc1ccc2c(c1)CN(C1CCC(=O)NC1=O)C2=O ; O=C(O)c1ccccc1C(F)(F)F ; O=C1CCC(N2Cc3cc(NC(=O)c4ccccc4C(F)(F)F)ccc3C2=O)C(=O)N1]

To a solution of 2-(trifluoromethyl)benzoic acid (88.0 mg, 463 umol, 1.20 eq) in dimethyl formamide (1.50 mL) was added O-(7-Azabenzotriazol-1-yl)-N,N,N,N-tetramethyluronium hexafluorophosphate (220 mg, 579 umol, 1.50 eq) and diisopropylethylamine (100 mg, 775 umol, 135 μL, 2.01 eq), the mixture was stirred at 0.5 h, then 3-(5-amino-1-oxoisoindolin-2-yl)piperidine-2,6-dione (100 mg, 386 umol, 1.00 eq) was added into the mixture. The mixture was stirred at 25° C. for 15.5 h. The pH was adjusted to around 6 by progressively adding formic acid and filtered. The filtrate was purified by Prep-HPLC (column: Phenomenex luna C18 150*25 mm*10 um; mobile phase: [water(FA)-ACN]; B %: 20%-50%, 9 min). The desired fraction was collected and lyophilized to give N-(2-(2,6-dioxopiperidin-3-yl)-1-oxoisoindolin-5-yl)-2-(trifluoromethyl)benzamide (Compound 137, 88.67 mg, 204 umol, 53% yield, 99% purity) as a grey solid.

¹H NMR (400 MHz, DMSO-d₆) δ=10.98 (s, 1H), 10.91 (s, 1H), 8.09 (s, 1H), 7.90-7.85 (m, 1H), 7.85-7.79 (m, 1H), 7.75 (br d, J=7.4 Hz, 2H), 7.72-7.66 (m, 2H), 5.10 (dd, J=5.1, 13.3 Hz, 1H), 4.51-4.43 (m, 1H), 4.37-4.29 (m, 1H), 3.01-2.83 (m, 1H), 2.61 (br d, J=17.0 Hz, 1H), 2.45-2.35 (m, 1H), 2.05-1.96 (m, 1H). MS (ESI) m/z 432.1 [M+H]⁺.

### Example 38. Synthesis of Compound 138

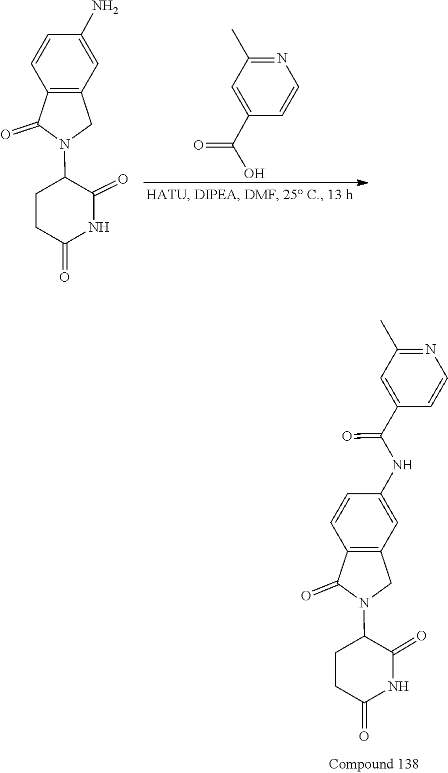

To a solution of 2-methylpyridine-4-carboxylic acid (79.3 mg, 578 umol, 1.50 eq) in dimethylformamide (0.500 mL) was added O-(7-azabenzotriazol-1-yl)-N,N,N,N-tetramethyluroniumhexafluorophosphate (219 mg, 578 umol, 1.50 eq) and N,N-diisopropylethylamine (149 mg, 1.16 mmol, 201 μL, 3.00 eq). The reaction mixture was stirred at 25° C. for 1 h. Then 3-(5-amino-1-oxo-isoindolin-2-yl)piperidine-2,6-dione (100 mg, 385 umol, 1.00 eq) was added and the reaction mixture was stirred at 25° C. for 12 h. The reaction mixture was added formic acid (2.00 mL) and filtered to give a filtrate. The filtrate was purified by Prep-HPLC (column: Phenomenex luna C18 150*25 mm*10 um; mobile phase: [water(formic acid)-acetonitrile]; B %: 37%-67%, 10.5 min) and lyophilized to give N-(2-(2,6-dioxopiperidin-3-yl)-1-oxoisoindolin-5-yl)-2-methylisonicotinamide (Compound 138, 44.45 mg, 103 umol, 26% yield, 99% purity, formate) as a blue solid.

¹H NMR (400 MHz, DMSO-d₆) δ=10.99 (s, 1H), 10.76 (s, 1H), 8.66 (d, J=5.1 Hz, 1H), 8.13 (s, 1H), 7.83 (dd, J=1.6, 8.3 Hz, 1H), 7.77-7.72 (m, 2H), 7.70-7.65 (m, 1H), 5.11 (dd, J=5.1, 13.2 Hz, 1H), 4.52-4.46 (m, 1H), 4.38-4.31 (m, 1H), 2.94-2.89 (m, 1H), 2.64 (br s, 1H), 2.59 (s, 3H), 2.53-2.43 (m, 1H), 2.06-1.98 (m, 1H). MS (ESI) m/z 379.0 [M+H]⁺.

### Example 39. Synthesis of Compound 139

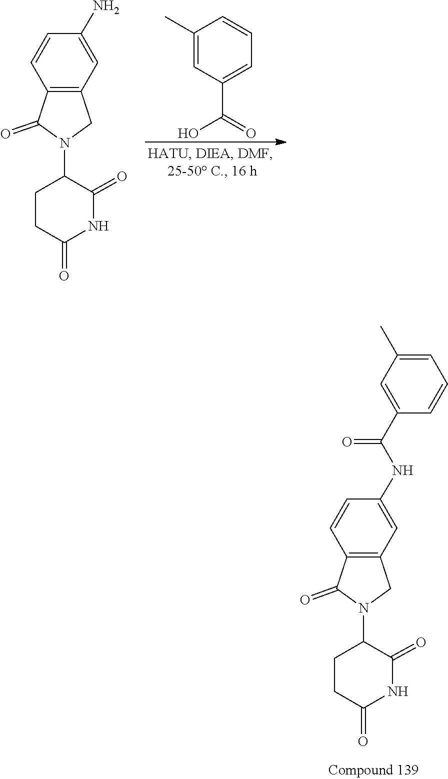

To a solution of 3-methylbenzoic acid (50.4 mg, 370 umol, 48.0 uL, 1.20 eq) in dimethyl formamide (1.50 mL) was added O-(7-Azabenzotriazol-1-yl)-N,N,N,N-tetramethyluronium hexafluorophosphate (176 mg, 463 umol, 1.50 eq) and diisopropylethylamine (79.8 mg, 617 umol, 107 μL, 2.00 eq) and 3-(5-amino-1-oxoisoindolin-2-yl)piperidine-2,6-dione (80.0 mg, 309 umol, 1.00 eq). The mixture was stirred at 25° C. for 4 h. Then the mixture was stirred at 50° C. for 12 h. The pH was adjusted to around 6 by progressively adding formic acid and filtered to give a solution, which was purified by Prep-HPLC (column: Welch Xtimate C18 150*25 mm*5 um; mobile phase: [water(FA)-ACN]; B %: 23%-53%, 10 min). The desired fraction was collected and the aqueous solution was lyophilized to give N-(2-(2,6-dioxopiperidin-3-yl)-1-oxoisoindolin-5-yl)-3-methylbenzamide (Compound 139, 57.93 mg, 152 umol, 49% yield, 99% purity) as an off-white solid.

¹H NMR (400 MHz, DMSO-d₆) δ=10.98 (s, 1H), 10.52 (s, 1H), 8.14 (s, 1H), 7.83 (br d, J=8.4 Hz, 1H), 7.80-7.73 (m, 2H), 7.71 (d, J=8.3 Hz, 1H), 7.47-7.38 (m, 2H), 5.10 (dd, J=4.9, 13.1 Hz, 1H), 4.53-4.41 (m, 1H), 4.38-4.26 (m, 1H), 2.99-2.85 (m, 1H), 2.60 (br d, J=17.6 Hz, 1H), 2.41 (s, 3H), 2.40-2.34 (m, 1H), 2.05-1.96 (m, 1H). MS (ESI) m/z 377.9 [M+H]⁺.

### Example 40. Synthesis of Compound 140

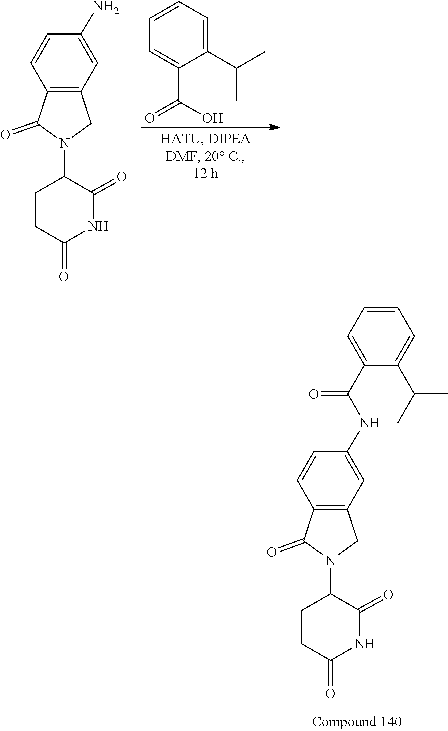 [SMILES: Nc1ccc2c(c1)CN(C1CCC(=O)NC1=O)C2=O ; CC(C)c1ccccc1C(=O)O ; CC(C)c1ccccc1C(=O)Nc1ccc2c(c1)CN(C1CCC(=O)NC1=O)C2=O]

To a solution of 2-isopropylbenzoic acid (76.0 mg, 462 umol, 1.20 eq), N,N-diisopropylethylamine (149 mg, 1.16 mmol, 201 μL, 3.00 eq) in dimethylformamide (1.00 mL) was added O-(7-azabenzotriazol-1-yl)-N,N,N′,N′-tetramethyluronium hexafluorophosphate (219 mg, 578 umol, 1.50 eq). The mixture was stirred at 20° C. for 10 min, then 3-(5-amino-1-oxoisoindolin-2-yl)piperidine-2,6-dione (100 mg, 385 umol, 1.00 eq) was added. The mixture was stirred at 20° C. for 12 h. The reaction mixture was diluted with water (30 mL), extracted with ethyl acetate (3×50 mL). The combined organic layers were dried over anhydrous sodium sulfate, filtered and concentrated under reduced pressure to give a residue. The residue was purified by Prep-HPLC (column: Phenomenex luna C18 150*25 mm*10 um; mobile phase: [water (formic acid)-acetonitrile]; B %: 27%-57%, 9 min) and lyophilized to afford N-(2-(2,6-dioxopiperidin-3-yl)-1-oxoisoindolin-5-yl)-2-isopropylbenzamide (Compound 140, 38.70 mg, 94.5 umol, 24% yield, 99% purity) as an off-white solid.

¹H NMR (400 MHz, DMSO-d₆) δ=10.98 (s, 1H), 10.73 (s, 1H), 8.16 (s, 1H), 7.76-7.71 (m, 1H), 7.71-7.67 (m, 1H), 7.47 (br d, J=3.6 Hz, 2H), 7.41 (d, J=7.3 Hz, 1H), 7.34-7.27 (m, 1H), 5.10 (dd, J=4.9, 13.4 Hz, 1H), 4.51-4.42 (m, 1H), 4.36-4.28 (m, 1H), 3.25-3.17 (m, 1H), 2.98-2.85 (m, 1H), 2.63 (br s, 1H), 2.44-2.36 (m, 1H), 2.05-1.96 (m, 1H), 1.22 (d, J=6.9 Hz, 6H). MS (ESI) m/z 406.1 [M+H]⁺.

### Example 41. Synthesis of Compound 141

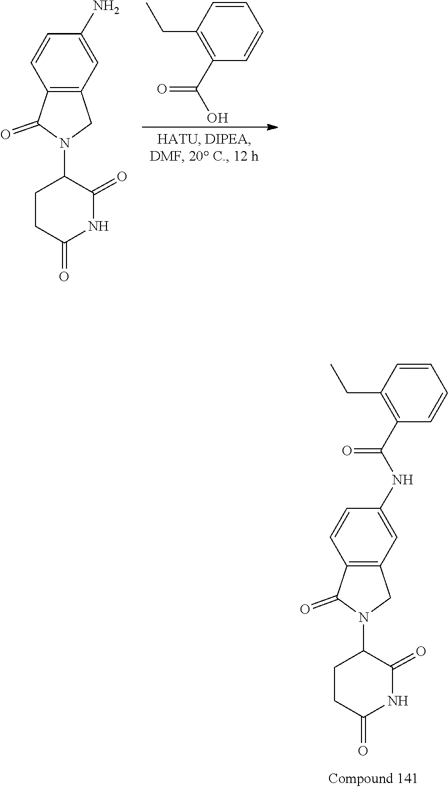 [SMILES: Nc1ccc2c(c1)CN(C1CCC(=O)NC1=O)C2=O ; CCc1ccccc1C(=O)O ; CCc1ccccc1C(=O)Nc1ccc2c(c1)CN(C1CCC(=O)NC1=O)C2=O]

To a solution of 2-ethylbenzoic acid (63.7 mg, 424 umol, 1.10 eq) in N,N-dimethylformamide (1.00 mL) was added N,N-diisopropylethylamine (150 mg, 1.16 mmol, 3.01 eq) and O-(7-azabenzotriazol-1-yl)-N,N,N′,N′-tetramethyluronium hexafluorophosphate (155 mg, 407 umol, 1.06 eq). The mixture was stirred at 20° C. for 3 min and then 3-(5-amino-1-oxo-isoindolin-2-yl)piperidine-2,6-dione (100 mg, 385 umol, 1.00 eq) was added. The reaction mixture was stirred at 20° C. for 12 h. The mixture solution was filtered. The filtrate was purified by Prep-HPLC (column: Phenomenex luna C18 150*25 mm*10 um; mobile phase: [water (FA)-ACN]; B %: 23%-53%, 10.5 min) and lyophilized to afford N-(2-(2,6-dioxopiperidin-3-yl)-1-oxoisoindolin-5-yl)-2-ethylbenzamide (Compound 141, 42.29 mg, 108 umol, 28% yield) as a white solid.

¹H NMR (400 MHz, DMSO-d₆) δ=10.98 (s, 1H), 10.69 (s, 1H), 8.15 (s, 1H), 7.73-7.68 (m, 2H), 7.47-7.45 (m, 2H), 7.37-7.33 (m, 2H), 5.12-5.07 (m, 1H), 4.48-4.44 (d, J=17.2 Hz, 1H), 4.34-4.30 (d, J=17.2 Hz, 1H), 2.89 (m, 1H), 2.76-2.72 (m, 2H), 2.52-2.49 (m, 1H), 2.38 (m, 1H), 2.02-2.00 (m, 1H), 1.19-1.16 (m, 3H). MS (ESI) m/z. 392.1 [M+H]⁺.

### Example 42. Synthesis of Compound 142

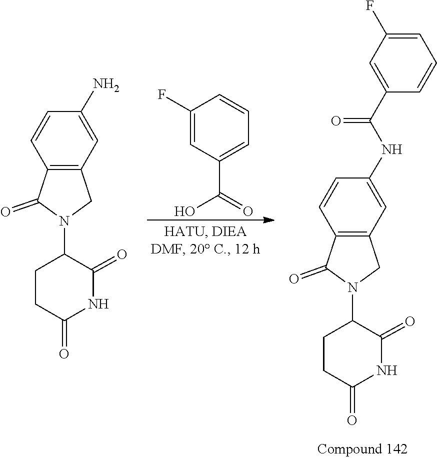 [SMILES: Nc1ccc2c(c1)CN(C1CCC(=O)NC1=O)C2=O ; O=C(O)c1cccc(F)c1 ; O=C1CCC(N2Cc3cc(NC(=O)c4cccc(F)c4)ccc3C2=O)C(=O)N1]

To a solution of 3-fluorobenzoic acid (32.4 mg, 231 umol, 1.20 eq), N,N-diisopropylethylamine (74.7 mg, 578 umol, 100 μL, 3.00 eq) in dimethylformamide (2.00 mL) was added O-(7-azabenzotriazol-1-yl)-N,N,N′,N′-tetramethyluronium hexafluorophosphate (110 mg, 289 umol, 1.50 eq). The mixture was stirred at 20° C. for 10 min. Then 3-(5-amino-1-oxoisoindolin-2-yl)piperidine-2,6-dione (50.0 mg, 192 umol, 1.00 eq) was added. The mixture was stirred at 20° C. for 12 h. The mixture was filtered. The filtrate was purified by Prep-HPLC (column: Phenomenex luna C18 150*25 mm*10 um; mobile phase: [water (formic acid)-acetonitrile]; B %: 19%-49%, 10.5 min) and lyophilized to give a crude product. The crude product was triturated with petroleum ether (4 mL) at 20° C. for 0.5 h and filtered again. The filter cake was diluted with water (10 mL) and lyophilized to afford N-(2-(2,6-dioxopiperidin-3-yl)-1-oxoisoindolin-5-yl)-3-fluorobenzamide (Compound 142, 20.63 mg, 53.5 umol, 27% yield, 99% purity) as a gray solid.

¹H NMR (400 MHz, DMSO-d₆) δ=10.99 (br s, 1H), 10.62 (s, 1H), 8.13 (s, 1H), 7.83 (br d, J=8.1 Hz, 2H), 7.80 (dd, J=2.1, 9.8 Hz, 1H), 7.73 (d, J=8.4 Hz, 1H), 7.61 (dt, J=5.9, 7.9 Hz, 1H), 7.48 (dt, J=1.9, 8.5 Hz, 1H), 5.10 (dd, J=5.1, 13.3 Hz, 1H), 4.52-4.44 (m, 1H), 4.36 (s, 1H), 2.99-2.86 (m, 1H), 2.64-2.59 (m, 1H), 2.39 (m, 1H), 2.05-1.98 (m, 1H). MS (ESI) m/z 382.1 [M+H]⁺.

### Example 43. Synthesis of Compound 143

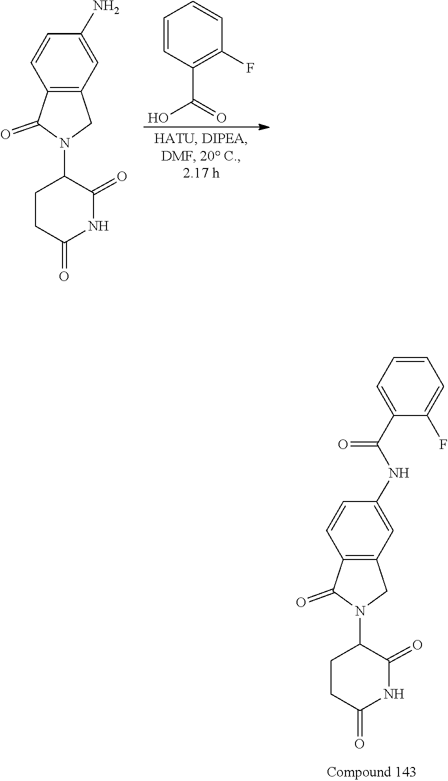 [SMILES: Nc1ccc2c(c1)CN(C1CCC(=O)NC1=O)C2=O ; O=C(O)c1ccccc1F ; O=C1CCC(N2Cc3cc(NC(=O)c4ccccc4F)ccc3C2=O)C(=O)N1]

To a solution of 2-fluorobenzoic acid (162 mg, 1.16 mmol, 3.00 eq) and N,N-diisopropylethylamine (299 mg, 2.31 mmol, 403 μL, 6.00 eq) in dimethyl formamide (3.00 mL) was added O-(7-azabenzotriazol-1-yl)-N,N,N,N-tetramethyluroniumhexafluorophosphate (440 mg, 1.16 mmol, 3.00 eq). The mixture was stirred at 20° C. for 10 min, and then 3-(5-amino-1-oxo-isoindolin-2-yl)piperidine-2,6-dione (100 mg, 386 umol, 1.00 eq) was added at 20° C. The resulting mixture was stirred at 20° C. for 2 h. The reaction mixture was added formic acid (2.00 mL) and filtered. The filtrate was purified by Prep-HPLC (column: Phenomenex luna C18 150*25 mm*10 um; mobile phase: [water(formic acid)-acetonitrile]; B %: 16%-46%, 9 min) and lyophilized to afforded N-(2-(2,6-dioxopiperidin-3-yl)-1-oxoisoindolin-5-yl)-2-fluorobenzamide (Compound 143, 61.83 mg, 139 umol, 36%, 96% purity, formate) as an off-white solid.

¹H NMR (400 MHz, DMSO-d₆) δ=10.99 (br s, 1H), 10.77 (s, 1H), 8.11 (s, 1H), 7.78-7.67 (m, 3H), 7.65-7.57 (m, 1H), 7.41-7.33 (m, 2H), 5.11 (dd, J=5.1, 13.3 Hz, 1H), 4.52-4.43 (m, 1H), 4.38-4.29 (m, 1H), 2.99-2.87 (m, 1H), 2.65-2.57 (m, 1H), 2.40 (br dd, J=4.5, 13.1 Hz, 1H), 2.08-1.96 (m, 1H). MS (ESI) m/z 382.0 [M+H]⁺.

### Example 44. Synthesis of Compound 144

### Step 1.

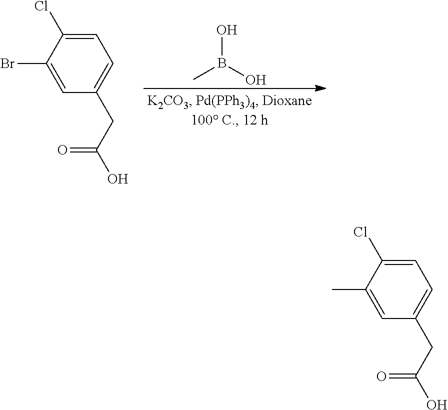

To a solution of 2-(3-bromo-4-chlorophenyl)acetic acid (1.00 g, 4.01 mmol, 1.00 eq) in dioxane (10.0 mL) were added methylboronic acid (720 mg, 12.0 mmol, 3.00 eq), potassium carbonate (1.66 g, 12.0 mmol, 3.00 eq) and tetrakis[triphenylphosphine]palladium(0) (463 mg, 401 umol, 0.100 eq). The mixture was stirred at 100° C. for 12 h under nitrogen atmosphere. The mixture was quenched with water (10 mL) and extracted with ethyl acetate (3×20 mL). The combined organic layers were washed with brine (10 mL), dried over anhydrous sodium sulfate, filtered and concentrated under reduced pressure to give a residue. The residue was purified by column chromatography (SiO₂, petroleum ether/ethyl acetate=1/0 to 3/1) to afford 2-(4-chloro-3-methylphenyl)acetic acid (200 mg, 867 umol, 22% yield, 80% purity) as a yellow solid.

¹H NMR (400 MHz, DMSO-d₆) δ=12.37 (br s, 1H), 7.33 (d, J=8.0, 1H), 7.23 (d, J=1.6 Hz, 1H), 7.10 (dd, J=2.0, 8.4 Hz, 1H), 3.54 (s, 2H), 2.30 (s, 3H).

### Step 2.

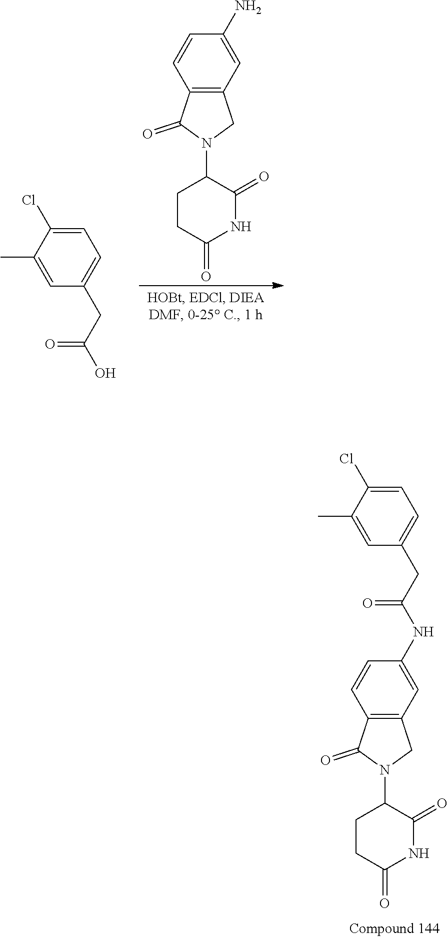 [SMILES: Cc1cc(CC(=O)O)ccc1Cl ; Nc1ccc2c(c1)CN(C1CCC(=O)NC1=O)C2=O ; Cc1cc(CC(=O)Nc2ccc3c(c2)CN(C2CCC(=O)NC2=O)C3=O)ccc1Cl]

To a solution of 2-(4-chloro-3-methylphenyl)acetic acid (37.5 mg, 203 umol, 1.20 eq) in dimethylformamide (1.00 mL) were added 3-(5-amino-1-oxo-isoindolin-2-yl)piperidine-2,6-dione (50.0 mg, 169 umol, 1.00 eq, hydrochloride) and N,N-diisopropylethylamine (65.6 mg, 508 umol, 88.4 uL, 3.00 eq). 1-hydroxybenzotriazole (22.9 mg, 170 umol, 1.00 eq) and 1-(3-dimethylaminopropyl)-3-ethylcarbodiimide hydrochloride (42.1 mg, 220 umol, 1.30 eq) were added to the solution at 0° C. The mixture was quenched by hydrochloric acid (1M, 5 mL). The mixture was extracted with a mixture of dichloromethane and iso-propanol (3:1, 3×10 mL). The combined organic layers were washed with brine (10 mL), dried over anhydrous sodium sulfate, filtered and concentrated under reduced pressure to give a residue. The residue was purified by Prep-TLC (SiO₂, dichloromethane/iso-propanol=10/1) to give a crude product. The crude product was purified by Prep-HPLC (column: Phenomenex Synergi C18 150*25 mm*10 um; mobile phase: [water(0.225% formic acid)-acetonitrile]; B %: 33%-53%, 10 min) and lyophilized to afford 2-(4-chloro-3-methylphenyl)-N-(2-(2,6-dioxopiperidin-3-yl)-1-oxoisoindolin-5-yl)acetamide (Compound 144, 31.41 mg, 73.02 umol, 22% yield, 99% purity) as a white solid.

¹H NMR (400 MHz, DMSO-d₆) δ=10.96 (s, 1H), 10.50 (s, 1H), 7.96 (s, 1H), 7.66 (br d, J=8.4 Hz, 1H), 7.60 (dd, J=1.2, 8.4 Hz, 1H), 7.36 (d, J=8.4 Hz, 1H), 7.31 (d, J=1.2 Hz, 1H), 7.18 (dd, J=2.0, 8.0 Hz, 1H), 5.07 (dd, J=5.2, 13.6 Hz, 1H), 4.46-4.37 (m, 1H), 4.32-4.24 (m, 1H), 3.67 (s, 2H), 2.97-2.83 (m, 1H), 2.63-2.55 (m, 1H), 2.44-2.34 (m, 1H), 2.32 (s, 3H), 2.03-1.93 (m, 1H). MS (ESI) m/z. 425.9 [M+H]⁺.

### Example 45. Synthesis of Compound 145

Compounds 145 was prepared using a method analogous to the syntheses of other compounds disclosed herein.

### Example 46. Synthesis of Compound 146

### Step 1.

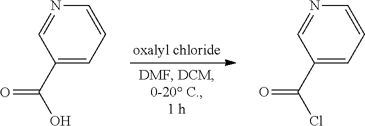 [SMILES: O=C(O)c1cccnc1 ; O=C(Cl)c1cccnc1]

To a solution of nicotinic acid (2.00 g, 16.3 mmol, 1.00 eq) in dichloromethane (20.0 mL) was added dimethyformamide (119 mg, 1.62 mmol, 125 μL, 0.100 eq) and oxalyl chloride (3.09 g, 24.4 mmol, 2.13 mL, 1.50 eq) at 0° C. slowly. The reaction mixture was stirred at 20° C. for 1 h. The reaction was concentrated under reduced pressure to afford nicotinoyl chloride (2.00 g, 14.1 mmol, 87% yield) as a white solid.

### Step 2.

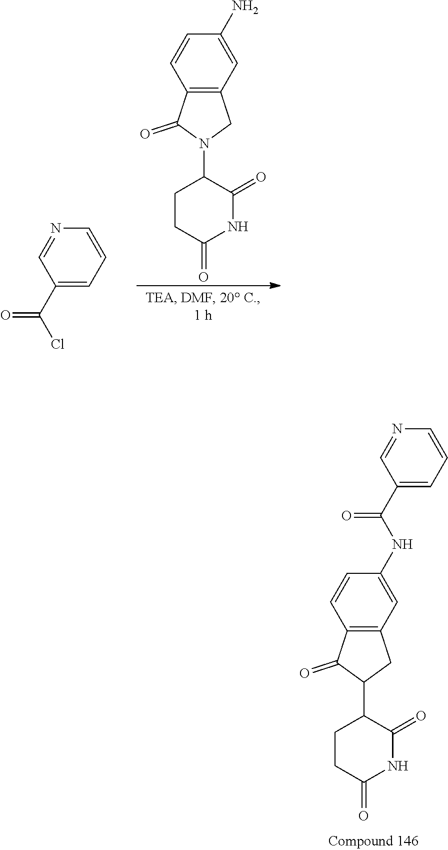 [SMILES: O=C(Cl)c1cccnc1 ; Nc1ccc2c(c1)CN(C1CCC(=O)NC1=O)C2=O ; O=C1CCC(C2Cc3cc(NC(=O)c4cccnc4)ccc3C2=O)C(=O)N1]

To a solution of 3-(5-amino-1-oxoisoindolin-2-yl)piperidine-2,6-dione (200 mg, 676 umol, 1.00 eq, hydrochloride) in dimethyformamide (2.00 mL) was added triethylamine (205 mg, 2.03 mmol, 282 μL, 3.00 eq) and nicotinoyl chloride (144 mg, 1.01 mmol, 7.36 uL, 1.50 eq). The reaction mixture was stirred at 20° C. for 1 h. The reaction was filtered. The filtrate was purified by Prep-HPLC (column: 3_Phenomenex Luna C18 75*30 mm*3 um; mobile phase: [water(0.05% hydrochloric acid)-acetonitrile]; B %: 5%-15%, 6 min) and lyophilized to afford N-(2-(2,6-dioxopiperidin-3-yl)-1-oxo-2,3-dihydro-1H-inden-5-yl)nicotinamide (Compound 146, 66.13 mg, 179.68 umol, 27% yield, 99% purity) as a yellow solid.

¹H NMR (400 MHz, DMSO-d₆) δ=11.26 (s, 1H), 10.99 (s, 1H), 9.41 (s, 1H), 8.98 (br d, J=4.5 Hz, 1H), 8.82 (br d, J=7.9 Hz, 1H), 8.18 (s, 1H), 8.06-7.85 (m, 2H), 7.75 (d, J=8.3 Hz, 1H), 5.11 (dd, J=5.0, 13.3 Hz, 1H), 4.49 (d, J=17.2 Hz, 1H), 4.34 (d, J=17.2 Hz, 1H), 3.00-2.81 (m, 1H), 2.61 (br d, J=16.8 Hz, 1H), 2.46-2.32 (m, 1H), 2.07-1.92 (m, 1H). MS (ESI) m/z 365.1 [M+H]⁺.

### Example 47. Synthesis of Compound 147

### Step 1.

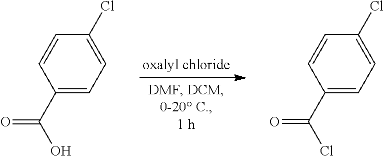 [SMILES: O=C(O)c1ccc(Cl)cc1 ; O=C(Cl)c1ccc(Cl)cc1]

To a solution of 4-chlorobenzoic acid (2.00 g, 12.8 mmol, 1.36 mL, 1 eq) in dichloromethane (20.0 mL) was added dimethyformamide (1.28 mmol, 98.3 uL, 0.100 eq) and oxalyl chloride (19.2 mmol, 1.68 mL, 1.50 eq) at 0° C. slowly. The reaction mixture was stirred at 20° C. for 1 h. The reaction was concentrated under reduced pressure to afford 4-chlorobenzoyl chloride (2.00 g, 11.4 mmol, 89% yield) as a white solid.

### Step 2.

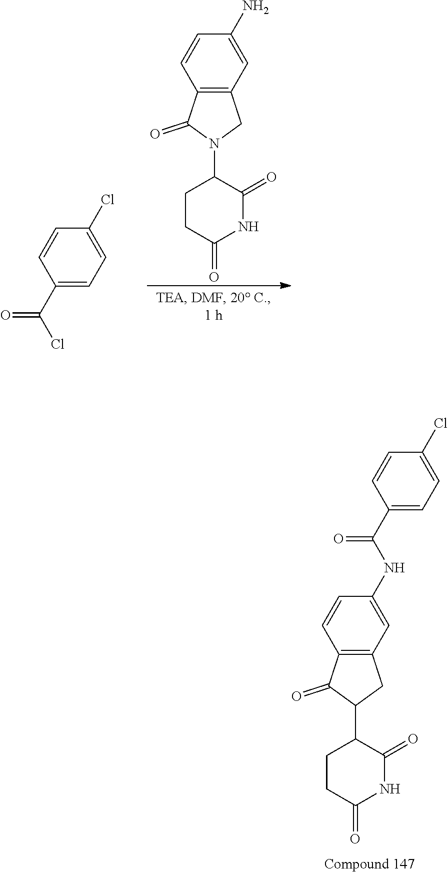 [SMILES: O=C(Cl)c1ccc(Cl)cc1 ; Nc1ccc2c(c1)CN(C1CCC(=O)NC1=O)C2=O ; O=C1CCC(C2Cc3cc(NC(=O)c4ccc(Cl)cc4)ccc3C2=O)C(=O)N1]

To a solution of 3-(5-amino-1-oxoisoindolin-2-yl)piperidine-2,6-dione (200 mg, 676 umol, 1.00 eq, hydrochloride) in dimethyformamide (2.00 mL) was added triethylamine (2.03 mmol, 282 μL, 3.00 eq) and 4-chlorobenzoyl chloride (118 mg, 676 umol, 1.00 eq). The reaction mixture was stirred at 20° C. for 1 h. The reaction was filtered. The filtrate was purified by Prep-HPLC (column: Waters Xbridge 150*25 mm*5 um; mobile phase: [water(10 mM ammonium bicarbonate)-acetonitrile]; B %: 27%-57%, 8 min) and lyophilized to afford 4-chloro-N-(2-(2,6-dioxopiperidin-3-yl)-1-oxo-2,3-dihydro-1H-inden-5-yl) benzamide (46 mg, 114.48 umol, 17% yield, 99% purity) as an off-white solid.

¹H NMR (400 MHz, DMSO-d₆) δ=11.2-10.8 (m, 1H), 10.8-10.3 (m, 1H), 8.14 (s, 1H), 8.07-7.98 (m, 2H), 7.83 (dd, J=1.5, 8.4 Hz, 1H), 7.72 (d, J=8.3 Hz, 1H), 7.64 (d, J=8.6 Hz, 2H), 5.10 (dd, J=5.1, 13.4 Hz, 1H), 4.48 (J=17.2, 1H), 4.33 (J=17.2, 1H), 2.99-2.86 (m, 1H), 2.68-2.63 (m, 1H), 2.40 (br dd, J=4.3, 13.2 Hz, 1H), 2.05-1.96 (m, 1H).

¹H NMR (400 MHz, HCl+DMSO-d₆) δ=11.0 (br s, 1H), 10.8 (s, 1H), 8.15 (br s, 1H), 8.09-8.02 (m, 2H), 7.89 (dd, J=1.4, 8.3 Hz, 1H), 7.69 (br d, J=8.4 Hz, 1H), 7.59 (br dd, J=2.1, 6.3 Hz, 2H), 5.07 (br d, J=5.4 Hz, 1H), 4.46 (J=17.2, 1H), 4.31 (J=17.2, 1H), 2.93-2.83 (m, 1H), 2.65-2.62 (m, 1H), 2.40-2.33 (m, 1H), 2.04-1.93 (m, 1H). MS (ESI) m/z 398.1 [M+H]⁺.

### Example 48. Synthesis of Compound 149

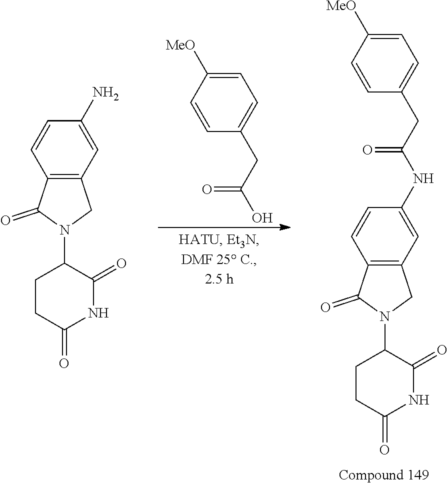 [SMILES: Nc1ccc2c(c1)CN(C1CCC(=O)NC1=O)C2=O ; COc1ccc(CC(=O)O)cc1 ; COc1ccc(CC(=O)Nc2ccc3c(c2)CN(C2CCC(=O)NC2=O)C3=O)cc1]

To a solution of 2-(4-methoxyphenyl)acetic acid (70.5 mg, 424 umol, 1.10 eq) in dimethylformamide (0.500 mL) were added O-(7-azabenzotriazol-1-yl)-N,N,N′,N′-tetramethyluronium (191 mg, 501 umol, 1.30 eq) and triethylamine (117 mg, 1.16 mmol, 161 μL, 3.00 eq). The mixture was stirred at 25° C. for 0.5 h. 3-(5-amino-1-oxoisoindolin-2-yl)piperidine-2,6-dione (100 mg, 386 umol, 1.00 eq) was added to the solution. The mixture was stirred at 25° C. for 2 h. The mixture was quenched with water (5 mL) and extracted with ethyl acetate (3×20 mL). The combined organic layers were washed with brine (2×5 mL), dried over anhydrous sodium sulfate, filtered and concentrated to give a residue. The mixture was purified by Prep-HPLC (column: Unisil 3-100 C18 Ultra 150*50 mm*3 um; mobile phase: [water (0.225% formic acid)-acetonitrile]; B %: 23%-53%, 10 min) to afford N-(2-(2,6-dioxopiperidin-3-yl)-1-oxoisoindolin-5-yl)-2-(4-methoxyphenyl)acetamide (29.44 mg, 70.81 umol, 18% yield, 98% purity) as a white solid.

¹H NMR (400 MHz, DMSO-d₆) δ=10.97 (s, 1H), 10.45 (s, 1H), 7.96 (s, 1H), 7.65 (br d, J=8.4 Hz, 1H), 7.60 (dd, J=1.2, 8.4 Hz, 1H), 7.26 (d, J=8.8 Hz, 2H), 6.89 (d, J=8.4 Hz, 2H), 5.07 (dd, J=5.2, 13.6 Hz, 1H), 4.41 (br d, J=17.2 Hz, 1H), 4.28 (br d, J=17.2 Hz, 1H), 3.73 (s, 3H), 3.61 (s, 2H), 2.96-2.85 (m, 1H), 2.59 (br d, J=17.2 Hz, 1H), 2.41-2.33 (m, 1H), 2.02-1.94 (m, 1H).

### Example 49. Synthesis of Compounds 148 and 150-153

Compounds 148 and 150-153 were prepared using methods analogous to the syntheses of other compounds disclosed herein.

### Example 50. Synthesis of 155

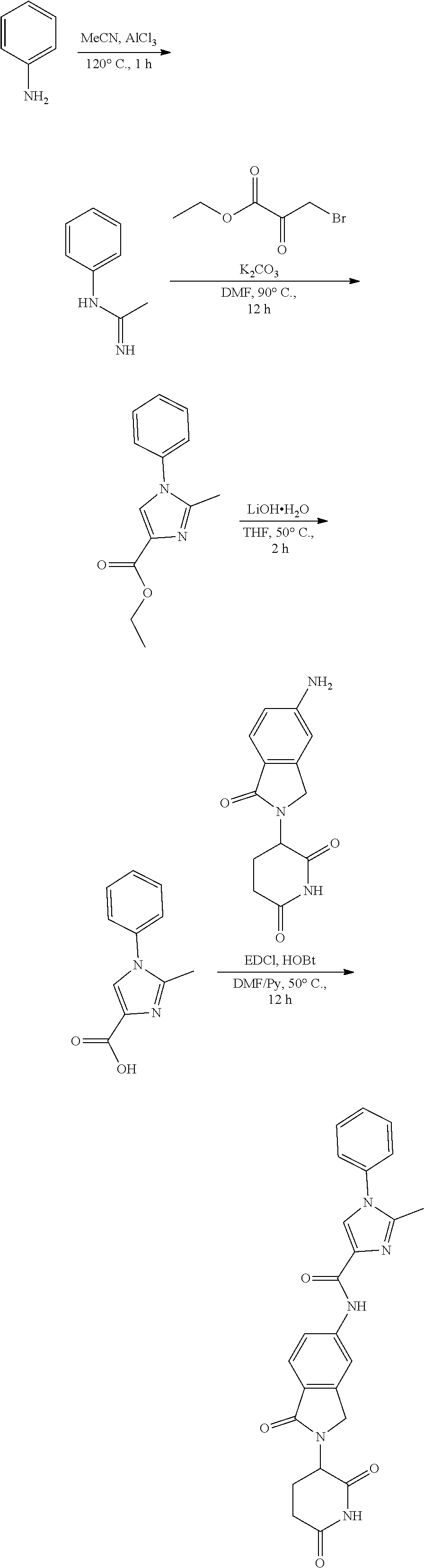

Step 1. To a solution of aniline (2.00 g, 21.4 mmol, 1.96 mL, 1.00 eq) in acetonitrile (881 mg, 21.4 mmol, 1.13 mL, 1.00 eq) was added aluminum trichloride (2.86 g, 21.4 mmol, 1.17 mL, 1.00 eq). The mixture was stirred at 120° C. for 1 hr. The reaction mixture was added ice-water (50 mL) at 0° C. and aqueous sodium hydroxide solution was added until pH>14. The aqueous phase was extracted with dichloromethane (100 mL). The combined organic phase was washed with brine (30 mL), dried with anhydrous sodium sulfate, filtered and concentrated in vacuum. The crude product was purified by reversed-phase HPLC (0.1% formic acid condition) and lyophilized to get N-phenylacetimidamide (2.00 g, 14.7 mmol, 69% yield, 99% purity) as a white solid. ¹H NMR (400 MHz, DMSO-d₆) δ=7.27-7.10 (m, 2H), 6.95-6.84 (m, 1H), 6.78 (br s, 1H), 6.65 (br d, J=6.8 Hz, 1H), 6.09 (br s, 1H), 1.95-1.57 (m, 3H).

Step 2. To a solution of N-phenylacetamidine (100 mg, 745.28 mol, 1.00 eq) and ethyl 3-bromo-2-oxo-propanoate (218 mg, 1.12 mmol, 139 μL, 1.50 eq) in dimethylformamide (2.00 mL) was added potassium carbonate (257 mg, 1.86 mmol, 2.50 eq). Then the reaction mixture was stirred at 90° C. for 12 h under nitrogen atmosphere. The reaction mixture was diluted with water (200 mL) and extracted with ethyl acetate (3×100 mL). The combined organic layers were washed with brine (20 mL), dried over sodium sulfate, filtered and concentrated under reduced pressure to give a residue. The residue was purified by reversed phase column chromatography (C18, 330 g; condition: water/acetonitrile=1/0 to 0/1, 0.1% formic acid) and lyophilized to got ethyl 2-methyl-1-phenyl-1H-imidazole-4-carboxylate (70.0 mg, 300 mol, 4% yield, 99% purity) as brown oil. ¹H NMR (400 MHz, DMSO-d₆) δ=7.98 (s, 1H), 7.60-7.54 (m, 2H), 7.54-7.48 (m, 3H), 4.26-4.21 (m, 2H), 2.29 (s, 3H), 1.28 (t, J=7.2 Hz, 3H).

Step 3. To a solution of ethyl 2-methyl-1-phenyl-imidazole-4-carboxylate (60.0 mg, 260 mol, 1.00 eq) in tetrahydrofuran (2.50 mL) and water (0.500 mL) was added lithium hydroxide (32.8 mg, 781 mol, 3.00 eq). Then the reaction mixture was stirred at 50° C. for 2 h. The reaction mixture was concentrated in vacuum to got 2-methyl-1-phenyl-imidazole-4-carboxylic acid (60.0 mg, crude) as brown oil. MS (ESI) m/z. 203.1 [M+H]⁺

Step 4. To a solution of 2-methyl-1-phenyl-imidazole-4-carboxylic acid (60.0 mg, 296 mol, 1.00 eq) and 3-(5-amino-1-oxo-isoindolin-2-yl)piperidine-2,6-dione (76.9 mg, 296 mol, 1.00 eq) in N,N-dimethylformamide (0.500 mL) and pyridine (0.500 mL) was added benzotriazol-1-ol (60.1 mg, 445 mol, 1.50 eq) and 1-(3-dimethylaminopropyl)-3-ethylcarbodiimide hydrochloride (85.3 mg, 445 mol, 1.50 eq). Then the reaction mixture was stirred at 50° C. for 12 h. The reaction mixture was filtered. The filtrate was purified by Prep-HPLC(column: Phenomenex luna C18 150*25 mm*10 um; mobile phase: [water(0.1% formic acid)-acetonitrile]; B %: 17%-47%, 10 min) and lyophilized to afford N-(2-(2,6-dioxopiperidin-3-yl)-1-oxoisoindolin-5-yl)-2-methyl-1-phenyl-1H-imidazole-4-carboxamide (13.5 mg, 30.0 mol, 10% yield, 99% purity) as a yellow solid. ¹H NMR (400 MHz, DMSO-d₆) δ=10.98 (s, 1H), 10.25 (s, 1H), 8.21 (s, 1H), 8.01 (s, 1H), 7.96 (dd, J=1.6, 8.4 Hz, 1H), 7.68 (d, J=8.4 Hz, 1H), 7.62-7.59 (m, 1H), 7.58 (d, J=3.2 Hz, 2H), 7.57-7.51 (m, 2H), 5.10 (dd, J=5.2, 13.2 Hz, 1H), 4.50-4.43 (m, 1H), 4.34-4.28 (m, 1H), 2.98-2.88 (m, 1H), 2.61 (br d, J=16.8 Hz, 1H), 2.48-2.41 (m, 1H), 2.38 (s, 3H), 2.05-1.97 (m, 1H). MS (ESI) m/z. 444.3 [M+H]⁺

### Example 51. Synthesis of 159

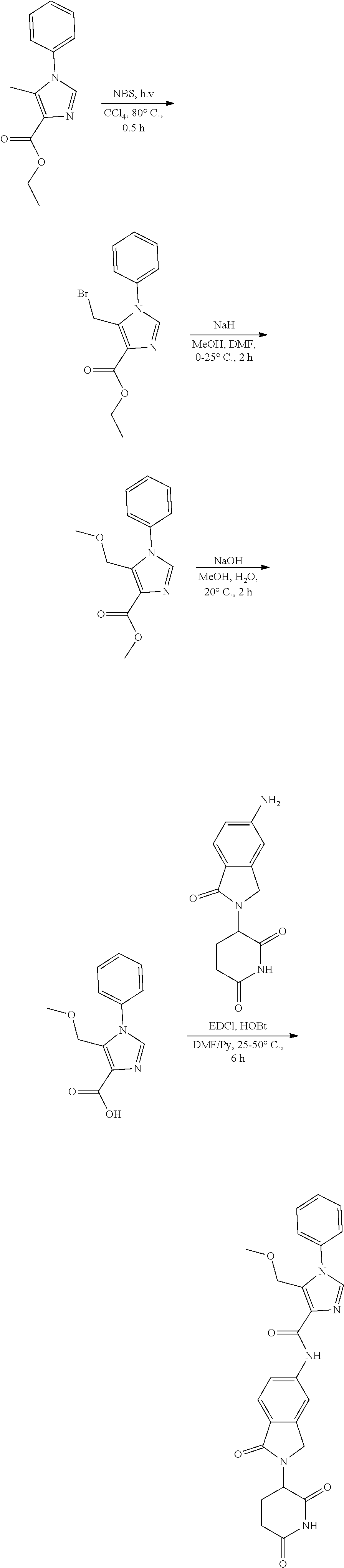

Step 1. To a solution of ethyl ethyl 5-methyl-1-phenyl-1H-imidazole-4-carboxylate (400 mg, 1.74 mmol, 1.00 eq) in tetrachloromethane (5.00 mL) was added N-Bromosuccinimide (309 mg, 1.74 mmol, 1.00 eq), the mixture was stirred at 80° C. for 30 min under irradiated. The reaction mixture was concentrated under reduced pressure to give a residue. The residue was purified by column chromatography (SiO2, Petroleum ether/Ethyl acetate=5/1 to 1/1) to afford ethyl 5-(bromomethyl)-1-phenyl-1H-imidazole-4-carboxylate (400 mg, 1.29 mmol, 74% yield) as a yellow solid. ¹H NMR (400 MHz, DMSO-d₆) δ=8.07 (s, 1H), 7.64-7.57 (m, 5H), 4.85 (s, 2H), 4.35-4.30 (m, 2H), 1.33 (t, J=7.2 Hz, 3H).

Step 2. To a solution of methyl ethyl 5-(bromomethyl)-1-phenyl-1H-imidazole-4-carboxylate (160 mg, 518 mol, 1.00 eq) in tetrahydrofuran (1.00 mL) and methanol (1.00 mL) was added sodium hydride (41.4 mg, 1.04 mmol, 60% purity, 2.00 eq) at 0° C., the mixture was stirred at 25° C. for 2 h. The reaction mixture was concentrated under reduced pressure to afford methyl 5-(methoxymethyl)-1-phenyl-1H-imidazole-4-carboxylate (120 mg, 487 mol, crude) as yellow oil. MS (ESI) m/z 247.3 [M+H]⁺

Step 3. To a solution of methyl 5-(methoxymethyl)-1-phenyl-1H-imidazole-4-carboxylate (120 mg, 487 mol, 1.00 eq) in methanol (1.00 mL) and water (1.00 mL) was added sodium hydroxide (78.0 mg, 1.95 mmol, 4.00 eq), the mixture was stirred at 20° C. for 2 h. The reaction mixture was concentrated under reduced pressure to give a residue. The residue was purified by reversed phase-HPLC (FA condition) to afford 5-(methoxymethyl)-1-phenyl-1H-imidazole-4-carboxylic acid (60.0 mg, 258 mol, 53% yield) as a white solid.

¹H NMR (400 MHz, DMSO-d₆) δ=8.12 (s, 1H), 7.61-7.54 (m, 5H), 4.61 (s, 2H), 3.15 (s, 3H). MS (ESI) m/z 255.1 [M+Na]+

Step 4. To a solution of 5-(methoxymethyl)-1-phenyl-1H-imidazole-4-carboxylic acid ((40.0 mg, 172 mol, 1.00 eq) in dimethylformamide (0.500 mL) and pyridine (0.500 mL) was added 1-(3-dimethylaminopropyl)-3-ethylcarbodiimide hydrochloride (66.0 mg, 344 mol, 2.00 eq), 1H-benzo[d][1,2,3]triazol-1-ol (46.6 mg, 344 mol, 2.00 eq) and 3-(5-amino-1-oxoisoindolin-2-yl)piperidine-2,6-dione (53.6 mg, 207 mol, 1.20 eq) at 25° C., the mixture was stirred at 50° C. for 6 h. The mixture was filtered to give filtrate The filtrate was purified by Prep-HPLC (column: Phenomenex luna C18 150*25 mm*10 um; mobile phase: [water(FA)-ACN]; gradient:25%-55% B over 10 min) to afford N-(2-(2,6-dioxopiperidin-3-yl)-1-oxoisoindolin-5-yl)-5-(methoxymethyl)-1-phenyl-1H-imidazole-4-carboxamide (19.1 mg, 43.0 mol, 14% yield) as a gray solid.

¹H NMR (400 MHz, DMSO-d₆) δ=10.98 (s, 1H), 10.37 (s, 1H), 8.24 (s, 1H), 8.16 (s, 1H), 7.94 (dd, J=1.6, 8.4 Hz, 1H), 7.68 (d, J=8.2 Hz, 1H), 7.61-7.58 (m, 4H), 7.57-7.54 (m, 1H), 5.10 (dd, J=5.2, 13.2 Hz, 1H), 4.75 (s, 2H), 4.50-4.42 (m, 1H), 4.36-4.29 (m, 1H), 3.20 (s, 3H), 2.98-2.86 (m, 1H), 2.64-2.57 (m, 1H), 2.45-2.34 (m, 1H), 2.07-1.97 (m, 1H). MS (ESI) m/z 474.2 [M+H]⁺

### Example 52. Synthesis of 158

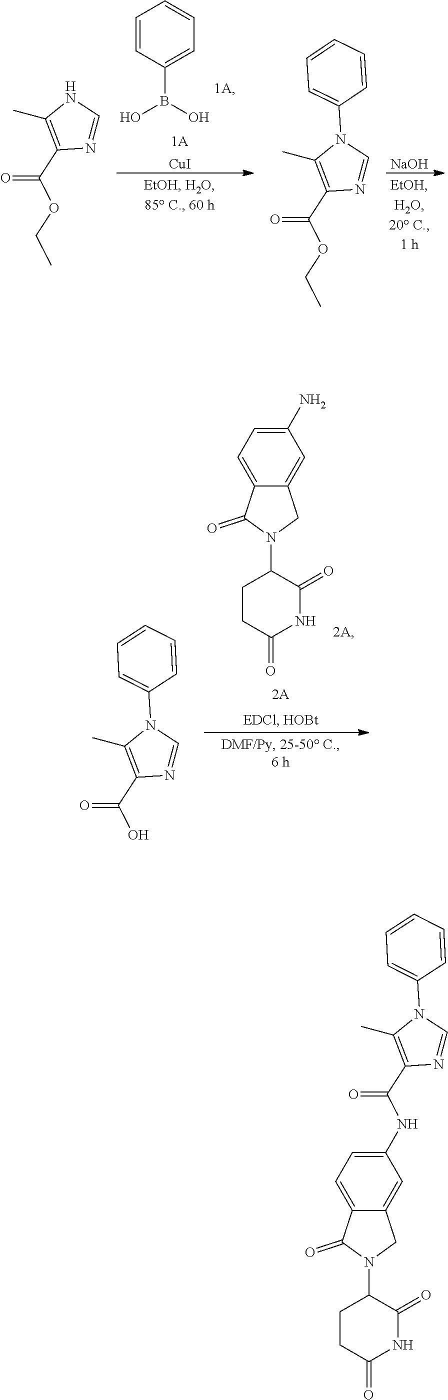

Step 1. To a solution of ethyl 5-methyl-1H-imidazole-4-carboxylate (500 mg, 3.24 mmol, 1.00 eq) in ethanol (3.00 mL) and water (3.00 mL) was added phenylboronic acid (633 mg, 5.19 mmol, 1.60 eq) and copper iodide (61.8 mg, 324 mol, 0.100 eq), the mixture was stirred at 85° C. for 60 h. The reaction mixture was diluted with water (30.0 mL) and exacted with ethyl acetate (3×30.0 mL). The organic phase was separated, washed with brine (2×10.0 mL), dried over sodium sulfate, filtered and concentrated under reduced pressure to give a residue. The residue was purified by column chromatography (SiO2, Petroleum ether/Ethyl acetate=10/1 to 1/1) to afford ethyl 5-methyl-1-phenyl-1H-imidazole-4-carboxylate (180 mg, 782 mol, 24% yield) as a green solid. ¹H NMR (400 MHz, DMSO-d₆) δ=8.55-7.69 (m, 1H), 7.63-7.52 (m, 3H), 7.48 (br d, J=7.6 Hz, 2H), 4.28 (br d, J=4.8 Hz, 2H), 2.40 (s, 3H), 1.30 (br t, J=5.8 Hz, 3H).

Step 2. To a solution of ethyl 5-methyl-1-phenyl-1H-imidazole-4-carboxylate (150 mg, 651 mol, 1.00 eq) in water (2.00 mL) and ethanol (2.00 mL) was added sodium hydroxide (104 mg, 2.61 mmol, 4.00 eq), the mixture was stirred at 20° C. for 1 h. The mixture was filtered to give filtrate, then was concentrated under reduced pressure to give a residue. The residue was purified by reversed phase-HPLC (0.1% FA condition) to afford 5-methyl-1-phenyl-1H-imidazole-4-carboxylic acid (80.0 mg, 396 mol, 61% yield) as a white solid.

MS (ESI) m/z 203.2 [M+H]⁺

Step 3. To a solution of 5-methyl-1-phenyl-1H-imidazole-4-carboxylic acid (60.0 mg, 297 mol, 1.00 eq) in dimethylformamide (0.500 mL) and pyridine (0.500 mL) was added 1-(3-dimethylaminopropyl)-3-ethylcarbodiimide hydrochloride (114 mg, 593 mol, 2.00 eq), 1H-benzo[d][1,2,3]triazol-1-ol (80.2 mg, 593 mol, 2.00 eq) and 3-(5-amino-1-oxoisoindolin-2-yl)piperidine-2,6-dione (92.3 mg, 356 mol, 1.20 eq) at 25° C., the mixture was stirred at 50° C. for 6 h. The mixture was filtered to give filtrate The filtrate was purified by Prep-HPLC (column: Phenomenex luna C18 150*25 mm*10 um; mobile phase: [water(FA)-ACN]; gradient:25%-55% B over 10 min) to afford N-(2-(2,6-dioxopiperidin-3-yl)-1-oxoisoindolin-5-yl)-5-methyl-1-phenyl-1H-imidazole-4-carboxamide (19.1 mg, 43.0 mol, 14% yield) as a gray solid. ¹H NMR (400 MHz, DMSO-d₆) δ=10.98 (s, 1H), 10.22 (s, 1H), 8.24 (s, 1H), 8.01 (s, 1H), 7.93 (dd, J=1.6, 8.2 Hz, 1H), 7.67 (d, J=8.2 Hz, 1H), 7.63-7.53 (m, 5H), 5.11 (dd, J=5.2, 13.2 Hz, 1H), 4.52-4.42 (m, 1H), 4.36-4.27 (m, 1H), 2.99-2.87 (m, 1H), 2.64-2.58 (m, 1H), 2.48 (br s, 3H), 2.41 (br dd, J=4.4, 13.0 Hz, 1H), 2.05-1.96 (m, 1H).

### Example 53. Synthesis of 154

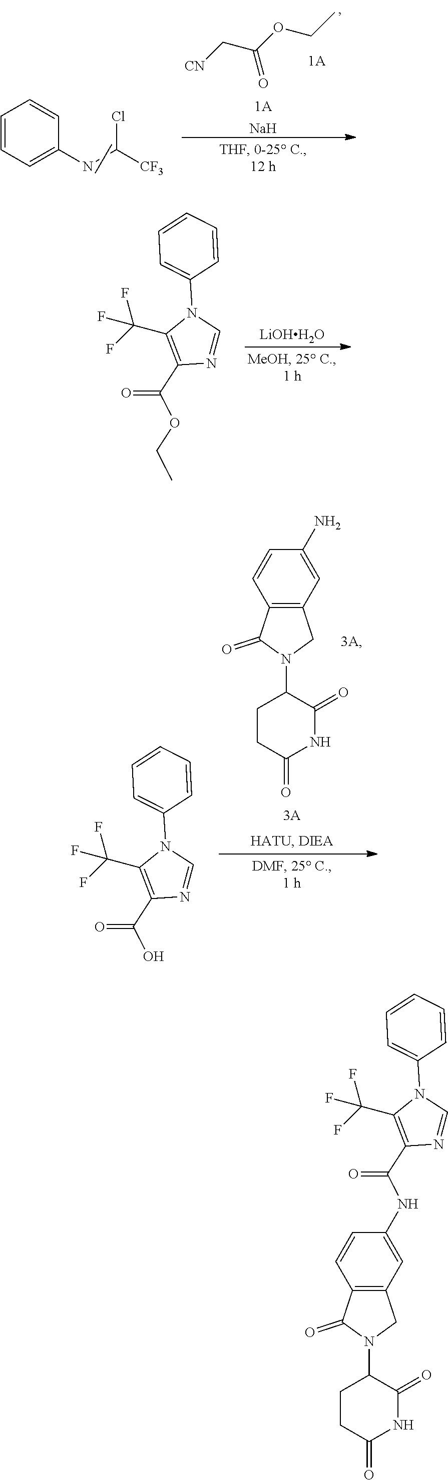

Step 1. To a solution of 2,2,2-trifluoro-N-phenylacetimidoyl chloride (500 mg, 2.41 mmol, 1.00 eq) in tetrahydrofuran (6.00 mL) was added sodium hydride (193 mg, 4.82 mmol, 60% purity, 2.00 eq) in portions at 0° C., the mixture was stirred at 0° C. for 0.5 h, then the ethyl 2-isocyanoacetate (327 mg, 2.89 mmol, 317 μL, 1.20 eq) was added, the mixture was stirred at 25° C. for 11.5 h. The reaction mixture was poured into water (80 mL) and extracted with ethyl acetate (3×40 mL), the combined organic phase was washed with brine (80 mL), dried over anhydrous sodium sulfate, filtered and concentrated in vacuum to give a residue. The residue was purified by silica gel chromatography (Petroleum ether:Ethyl acetate=1/0 to 3/1) to afford ethyl 1-phenyl-5-(trifluoromethyl)-1H-imidazole-4-carboxylate (500 mg, 1.76 mmol, 73% yield) as yellow oil. ¹H NMR (400 MHz, DMSO-d₆) δ=8.24-8.20 (m, 1H), 7.63-7.54 (m, 5H), 4.37-4.30 (m, 2H), 1.35-1.28 (m, 3H).

Step 2. To a solution of ethyl 1-phenyl-5-(trifluoromethyl)-1H-imidazole-4-carboxylate (500 mg, 1.76 mmol, 1.00 eq) in methanol (6.00 mL) was added lithium hydroxide monohydrate (221 mg, 5.28 mmol, 3.00 eq), the mixture was stirred at 25° C. for 1 h. The reaction mixture was poured into water (80 mL) and extracted with ethyl acetate (3×60 mL), the aqueous phase was adjust pH=1-2 with 1 M hydrochloric acid and extracted with ethyl acetate (3×60 mL), the combined organic phase was washed with brine (100 mL), dried with anhydrous sodium sulfate, filtered and concentrated in vacuum to give 1-phenyl-5-(trifluoromethyl)-1H-imidazole-4-carboxylic acid (150 mg, 586 umol, 33% yield) was obtained as a white solid.

Step 3. To a solution of 1-phenyl-5-(trifluoromethyl)-1H-imidazole-4-carboxylic acid (150 mg, 586 umol, 1.00 eq), 2-(7-azabenzotriazol-1-yl)-N,N,N,N-tetramethyluroniumhexafluorophosphate (334 mg, 878 umol, 1.50 eq) and diisopropylethylamine (227 mg, 1.76 mmol, 306 μL, 3.00 eq) in dimethyl formamide (2.00 mL) was added 3-(5-amino-1-oxoisoindolin-2-yl)piperidine-2,6-dione (152 mg, 586 umol, 1.00 eq), the mixture was stirred at 25° C. for 1 h, the reaction mixture was filtered to give a filtrate, the filtrate was purified by prep-HPLC (column: Phenomenex Luna C18 150×25 mm×10 um; mobile phase: [water(FA)-ACN]; B %: 30%-60%, 10 min) to afford N-(2-(2,6-dioxopiperidin-3-yl)-1-oxoisoindolin-5-yl)-1-phenyl-5-(trifluoromethyl)-1H-imidazole-4-carboxamide (63.6 mg, 128 umol, 22% yield) as a white solid. ¹H NMR (400 MHz, DMSO-d₆) δ=11.05-10.94 (m, 1H), 10.82-10.73 (m, 1H), 8.39-8.32 (m, 1H), 8.26-8.19 (m, 1H), 7.94-7.84 (m, 1H), 7.75-7.67 (m, 1H), 7.65-7.55 (m, 5H), 5.15-5.08 (m, 1H), 4.51-4.44 (m, 1H), 4.38-4.30 (m, 1H), 2.98-2.87 (m, 1H), 2.66-2.57 (m, 1H), 2.47-2.33 (m, 1H), 2.05-1.98 (m, 1H). MS (ESI) m/z 498.0 [M+H]⁺

### Example 54. Synthesis of 157

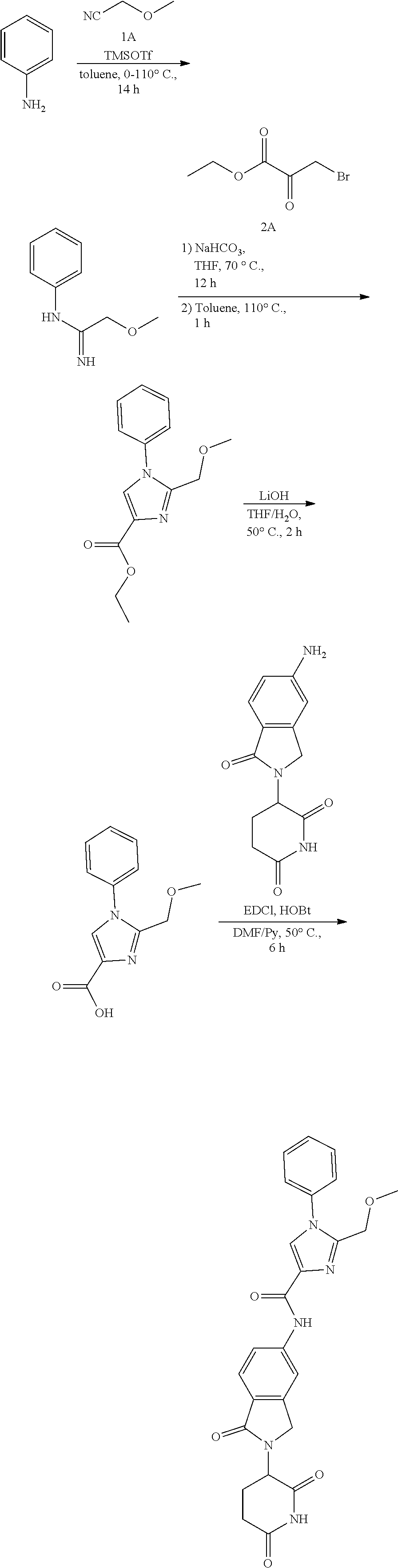

Step 1. To a solution of aniline (1.57 g, 16.9 mmol, 1.54 mL, 1.20 eq) and 2-methoxyacetonitrile (1.00 g, 14.1 mmol, 1.05 mL, 1.00 eq) in toluene (10.0 mL) was added trimethylsilyl trifluoromethanesulfonate (3.75 g, 16.9 mmol, 3.05 mL, 1.20 eq) at 0° C. The mixture was stirred at 110° C. for 14 h under nitrogen atmosphere. The reaction was cooled to 0° C. and water (5 mL) was added dropwise. The reaction mixture was diluted and extracted with ethyl acetate (3×50 mL). The organic layer was washed with saturated sodium bicarbonate aqueous solution. The organic layer was dried over magnesium sulfate, filtered and concentrated in vacuum to get a residue. The residue was purified by reversed-phase HPLC (column: spherical C18, 20-45 um, 100 Å, SW 120, mobile phase: [water (0.1% formic acid)-acetonitrile]) and lyophilized to get 2-methoxy-N-phenyl-acetamidine (2.00 g, 12.2 mmol, 87% yield) as brown oil. ¹H NMR (400 MHz, CDCl₃) δ=7.33 (t, J=7.8 Hz, 2H), 7.08-7.03 (m, 1H), 6.93 (d, J=7.6 Hz, 2H), 4.14 (s, 2H), 3.46 (s, 3H). MS (ESI) m/z 165.0 [M+H]⁺

Step 2. To a solution of ethyl 3-bromo-2-oxo-propanoate (1.43 g, 7.31 mmol, 913 μL, 1.20 eq), 2-methoxy-N-phenyl-acetamidine (1.00 g, 6.09 mmol, 1.00 eq) in tetrahydrofuran (15.0 mL) was added sodium bicarbonate (1.02 g, 12.2 mmol, 474 μL, 2.00 eq). The mixture was stirred at 70° C. for 12 h. The mixture was concentrated. The mixture was stirred in toluene (10.0 mL) and acetic acid (1.00 mL) at 110° C. for 1 h. The mixture was concentrated under reduced pressure. The mixture was purified by reversed-phase HPLC (column: spherical C18, 20-45 um, 100 Å, SW 120, mobile phase: [water(0.1% formic acid)-acetonitrile]) and lyophilized to give ethyl 2-(methoxymethyl)-1-phenyl-1H-imidazole-4-carboxylate (250 mg, crude) as a brown solid. ¹H NMR (400 MHz, CDCl₃) δ=7.81 (s, 1H), 7.54-7.45 (m, 5H), 4.46-4.40 (m, 4H), 3.37 (s, 3H), 1.41 (t, J=7.2 Hz, 3H). MS (ESI) m/z 261.0 [M+H]⁺

Step 3. To a solution of ethyl 2-(methoxymethyl)-1-phenyl-imidazole-4-carboxylate (200 mg, 768 umol, 1.00 eq) in water (0.300 mL) and tetrahydrofuran (1.50 mL) was added lithium hydroxide (55.2 mg, 2.31 mmol, 3.00 eq). The mixture was stirred at 50° C. for 2 h. The mixture was adjusted to pH=5-6 with formic acid and filtered. The filtrate was purified by reversed-phase HPLC (column: spherical C18, 20-45 um, 100 Å, SW 80, mobile phase: [water (0.1% formic acid)-acetonitrile) and lyophilized to give 2-(methoxymethyl)-1-phenyl-1H-imidazole-4-carboxylic acid (70.0 mg, 301 umol, 39% yield) as a brown solid. ¹H NMR (400 MHz, DMSO-d₆) δ=8.04 (s, 1H), 7.57-7.54 (m, 4H), 7.53-7.49 (m, 1H), 4.35 (s, 2H), 3.19 (s, 3H). MS (ESI) m/z 233.0 [M+H]⁺

Step 4. To a solution of 2-(methoxymethyl)-1-phenyl-imidazole-4-carboxylic acid (70.0 mg, 301 umol, 1.00 eq) in N,N-dimethyl formamide (1.00 mL) and pyridine (1.00 mL) was added 1-(3-dimethylaminopropyl)-3-ethylcarbodiimide hydrochloride (115 mg, 603 umol, 2.00 eq) and 1-hydroxybenzotriazole (81.5 mg, 603 umol, 2.00 eq). After 15 min, 3-(5-amino-1-oxo-isoindolin-2-yl)piperidine-2,6-dione (85.9 mg, 331 umol, 1.10 eq) was added to the mixture. The mixture was stirred at 50° C. for 6 h. The mixture was filtered. The filtrate was purified by reversed phase HPLC (column: spherical C 18, 20-45 um, 100 Å, SW 40, mobile phase: [water (0.1% formic acid)-acetonitrile) and lyophilized to afford N-(2-(2,6-dioxopiperidin-3-yl)-1-oxoisoindolin-5-yl)-2-(methoxymethyl)-1-phenyl-1H-imidazole-4-carboxamide (18.0 mg, 36.9 mol, 12% yield, 97% purity) as a yellow solid. ¹H NMR (400 MHz, DMSO-d₆) δ=10.98 (br s, 1H), 10.32 (s, 1H), 8.21 (s, 1H), 8.16 (s, 1H), 7.96 (dd, J=1.6, 8.4 Hz, 1H), 7.68 (d, J=8.4 Hz, 1H), 7.63-7.57 (m, 4H), 7.56-7.50 (m, 1H), 5.10 (dd, J=5.2, 13.2 Hz, 1H), 4.50-4.44 (m, 1H), 4.43 (s, 2H), 4.35-4.28 (m, 1H), 3.24 (s, 3H), 2.98-2.86 (m, 1H), 2.63-2.57 (m, 1H), 2.45-2.37 (m, 1H), 2.05-1.96 (m, 1H). MS (ESI) m/z 474.1 [M+H]⁺

### Example 55. Synthesis of 156

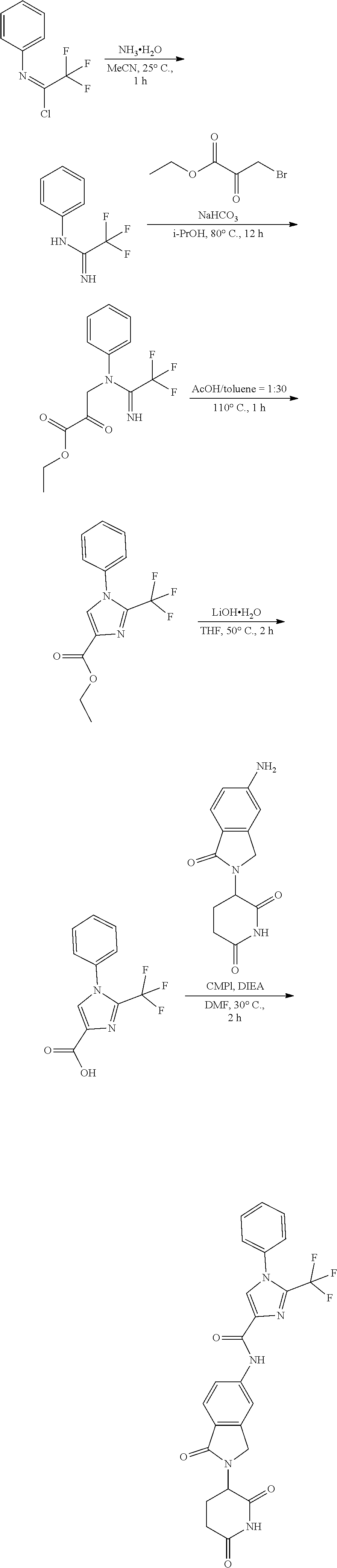

Step 1. To a solution of (E)-2,2,2-trifluoro-N-phenylacetimidoyl chloride (10.0 g, 48.1 mmol, 1.00 eq) in acetonitrile (100 mL) was added ammonium hydroxide (20.9 g, 149 mmol, 23.0 mL, 25% purity, 6.00 eq). Then the reaction mixture was stirred at 25° C. for 12 h. The reaction mixture was added into acetonitrile (300 mL) and filtered. The filtrate was concentrated in vacuum. The residue was lyophilized to get 2,2,2-trifluoro-N-phenylacetimidamide (8.00 g, 39.1 mmol, 92% purity) as a yellow solid. ¹H NMR (400 MHz, DMSO-d₆) δ=7.34 (t, J=7.6 Hz, 2H), 7.09-6.94 (m, 3H), 6.85 (br d, J=7.6 Hz, 2H).

Step 2. To a solution of 2,2,2-trifluoro-N-phenylacetimidamide (8.00 g, 42.5 mmol, 1.00 eq) and ethyl 3-bromo-2-oxo-propanoate (12.4 g, 63.7 mmol, 7.97 mL, 1.50 eq) in isopropanol (120 mL) was added saturated sodium bicarbonate (8.93 g, 106 mmol, 4.14 mL, 2.50 eq). Then the reaction mixture was stirred at 80° C. for 12 h under nitrogen atmosphere. The reaction mixture was filtered. The filtrate was concentrated in vacuum to get ethyl 2-oxo-3-(2,2,2-trifluoro-N-phenylacetimidamido)propanoate (13 g, crude) as brown oil.

Step 3. To a solution of ethyl 2-oxo-3-(2,2,2-trifluoro-N-phenylacetimidamido)propanoate (2.00 g, 6.62 mmol, 1.00 eq) in acetic acid (1.10 mL) and toluene (40.0 mL). Then the reaction mixture was stirred at 110° C. for 1 h under nitrogen atmosphere. The reaction mixture was concentrated in vacuum. The residue was purified by reversed phase column chromatography (C18, 330 g; condition: water/acetonitrile=1/0 to 0/1, 0.1% ammoniumhydroxide) and lyophilized to get ethyl 1-phenyl-2-(trifluoromethyl)-1H-imidazole-4-carboxylate (1.20 g, 2.11 mmol, 31% yield, 50% purity) as brown oil. ¹H NMR (400 MHz, DMSO-d₆) δ=8.43 (s, 1H), 7.61-7.56 (m, 5H), 4.34-4.30 (m, 2H), 1.30 (s, 3H). (¹H NMR and 2D NMR came from pilot run)

Step 4. To a solution of ethyl 1-phenyl-2-(trifluoromethyl)-1H-imidazole-4-carboxylate (800 mg, 2.81 mmol, 1.00 eq) in tetrahydrofuran (10.0 mL) and water (1.00 mL) was added lithium hydroxide (354 mg, 8.44 mmol, 3.00 eq). Then the reaction mixture was stirred at 50° C. for 2 h. The reaction mixture was concentrated in vacuum. The residue was diluted with water (100 mL) and extracted with ethyl acetate (3×50 mL). The aqueous layers were adjusted pH to 5-6 by hydrochloric acid (1 M, 10 mL) and extracted with ethyl acetate (3×50 mL). The combined organic layers were washed with brine (3×20 mL), dried over anhydrous sodium sulfate, filtered and concentrated under reduced pressure to give 1-phenyl-2-(trifluoromethyl)-1H-imidazole-4-carboxylic acid (300 mg, 1.05 mmol, 37% yield, 90% purity) as yellow oil. MS (ESI) m/z. 257.1 [M+H]⁺

Step 5. To a solution of 1-phenyl-2-(trifluoromethyl)-1H-imidazole-4-carboxylic acid (100 mg, 390 mol, 1.00 eq), 2-chloro-1-methyl-pyridin-1-ium; iodide (149 mg, 585 mol, 1.50 eq) and 3-(5-amino-1-oxo-isoindolin-2-yl)piperidine-2,6-dione (101 mg, 390 mol, 1.00 eq) in N,N-dimethylformamide (5.00 mL) was added N,N-diisopropylethylamine (151 mg, 1.17 mmol, 203 L, 3.00 eq). Then the reaction mixture was stirred at 30° C. for 2 h. The reaction mixture was adjusted pH to 5-6 by formic acid (0.3 mL) and filtered. The filtrate was purified by reversed phase column chromatography (C18, 80 g; condition: water/acetonitrile=1/0 to 0/1, 0.1% formic acid) and lyophilized to afford N-(2-(2,6-dioxopiperidin-3-yl)-1-oxoisoindolin-5-yl)-1-phenyl-2-(trifluoromethyl)-1H-imidazole-4-carboxamide (10.4 mg, 20.1 mol, 5% yield, 96% purity) as a white solid. ¹H NMR (400 MHz, DMSO-d₆) δ=10.99 (s, 1H), 10.45 (s, 1H), 8.42 (s, 1H), 8.19 (s, 1H), 7.95 (dd, J=1.6, 8.4 Hz, 1H), 7.71 (d, J=8.4 Hz, 1H), 7.62 (s, 5H), 5.11 (dd, J=5.4, 13.6 Hz, 1H), 4.51-4.44 (m, 1H), 4.36-4.29 (m, 1H), 2.98-2.88 (m, 1H), 2.61 (br d, J=17.2 Hz, 1H), 2.39 (br d, J=4.4 Hz, 1H), 2.04-1.99 (m, 1H). MS (ESI) m/z. 498.2 [M+H]⁺

### Example 56. CK1α NanoBiT Assay

HEK293 clonal line with CRISPR KI HiBiT tag on CSNK1A1 and stably expressing LgBiT protein was obtained from Promega (Madison, WI). Cells were plated at 5000 cells per well using Multiflo (BioTek) in 384-well white solid bottom plates (Corning, 3570BC) in 25 ml volume in DMEM media (DMEM, high glucose, HEPES, no phenol red (ThermoFisher Scientific, 21063029)) containing 10% FBS (Corning, 35-075-CV), 1% Penicillin/Streptomycin ((ThermoFisher Scientific, 15140-122), and 0.2% Endurazine (Nano-Glo Endurazine Live Cell Substrate (Promega, N2571)). Cells were incubated for 16 hours at 37C, 5% CO₂. Depending on experiment 25 or 75 nL of a compound at 10 mM were added into the plate using an Echo®650 liquid handler (Labcyte) to achieve final concentration of 10 or 30 mM in wells. Cells were incubated at 37° C., 5% CO₂ for 24 hours followed by signal was read on Pherastar FSX using “LUM plus” optic module.

Data analysis was performed using Scinamic (Scinamic, Cambridge, MA).

Luminescence response (R) was calculated by the formula: response=100*(S−N)/(P−N), where S is the signal of the well, N and P the mean negative and positive control values, respectively, of the same plate. The luminescence response was then fitted in Scinamic using a 3-parameter agonist logistic fit (hillslope=1, EC50>0, top/bottom unconstrained).

DC₅₀ data are reported in Table 2 for compounds in Table 1. In Table 2 below, According to the code, A represents a DC₅₀ value of ≤0.1 μM, B represents a DC₅₀ value>0.1 μM and ≤1 μM, C represents an DC₅₀ value>1 μM.

TABLE 2
CK1α data

| Compound | CK1α DC50 (μM) |
|---|---|
| Number | 24 h |
| 101 | C |
| 102 | B |
| 103 | B |
| 104 | B |
| 105 | C |
| 106 | B |
| 107 | B |
| 108 | C |
| 109 | A |
| 110 | C |
| 111 | C |
| 112 | C |
| 113 | B |
| 114 | C |
| 115 | C |
| 116 | C |
| 117 | A |
| 118 | C |
| 119 | C |
| 120 | A |
| 121 | A |
| 122 | A |
| 123 | B |
| 124 | B |
| 125 | A |
| 126 | A |
| 127 | A |
| 128 | C |
| 129 | A |
| 130 | B |
| 131 | A |
| 132 | B |
| 133 | B |
| 134 | B |
| 136 | B |
| 137 | A |
| 138 | C |
| 139 | A |
| 140 | B |
| 141 | B |
| 142 | A |
| 143 | B |
| 144 | B |
| 145 | A |
| 146 | C |
| 147 | A |
| 148 | A |
| 149 | B |
| 150 | B |
| 151 | B |
| 152 | C |
| 153 | C |
| 154 | A |
| 155 | A |
| 156 | A |
| 157 | A |
| 158 | A |
| 159 | B |

### Example 57. Western Blot Assay

HEK293 cell line was purchased from ATCC (CRL-1573). HEK293 CRBN knock out (k/o) cell line (B2) was generated internally using CRISPR/Cas9 method and clonally expanded. Cells were plated at 2×10⁵ cells per well in 6-well tissue culture plates (VWR) in 2 ml of DMEM media (Gibco) containing 10% FBS (Gibco), and incubated for 16 hours at 37° C., 5% CO₂. Compounds were added to final concentration of 0.1 μM, 1 μM, 10 μM (DMSO concentration was kept constant at 0.1%), following incubation at 37° C., 5% CO₂ for additional 24 hours. Cell lysis was performed using RIPA buffer (Pierce) containing Halt™ Protease Inhibitor Cocktail (ThermoFisher Scientific). Lysates were boiled at 95° C. for 10 minutes and 12 μg of protein lysate per sample was resolved by SDS-PAGE using 12% gels (BioRad) and transferred to nitrocellulose membrane (BioRad). Membranes were blocked using LI-COR blocking buffer (LI-COR) at room temperature for 1 hour, followed by overnight incubation with rabbit anti-CK1a (Abcam ab206652), rabbit anti-CRBN (Sigma HPA045910) and mouse anti-α-tubulin (Sigma T9026) antibodies at 4° C. Secondary antibodies (LI-COR) were added for 1 hour at room temperature, followed by imaging with LI-COR imaging system Odyssey® CLx.

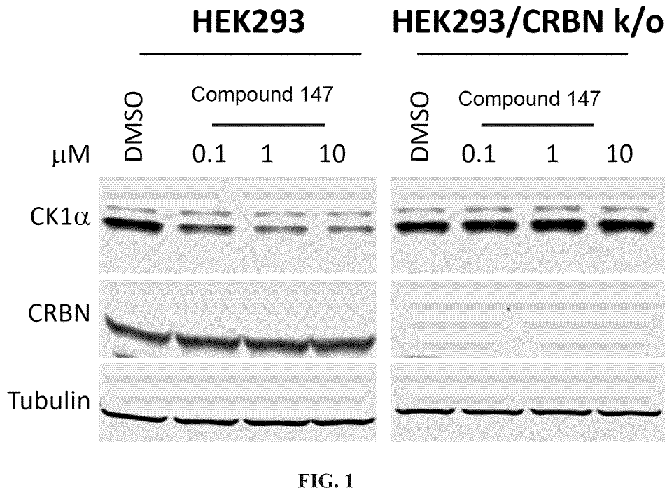

Exemplary Western Blots obtained from the protocol is shown in FIG. 1.

### Example 58. HTRF CRBN & Ternary Complex Assay

### HTRF Binding Assay

Binding of test compounds to CRBN/DDB1 was monitored in an HTRF assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. In this assay, compounds displace the fluorescently labeled thalidomide-based probe bound to CRBN and fluorescence is monitored with increasing compound concentration. Assays were conducted in Greiner white 384 well HiBase plates (Cat. No 784075-25) in 10 μL total volume. A one pot detection solution of CRBN/DDB1 (2.5 nM), anti-His Terbium Cryptate Gold (1×, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Test compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM lenalidomide (Selleckchem Cat no. S1029) was transferred into the active-control wells for the assay and 10 nL of DMSO was transferred into the negative control wells. The assay was then allowed to incubate for 30 min at ambient temperature after transferring the test compounds. Plate measurements were taken on a Pherastar FSX (BMG Labtech, Germany) using the HTRF Red filter (Ex. 337 nm, eml: 620 nm, em2: 665 nm) (Flashes: 50, Integration time: 60-400 μs, Z-height: 10 mm, Ratio-multiplier: 10,000). Analysis, HTRF ratio and IC₅₀ values were derived using KNIME analytics (KNIME Zurich) transformation and fitting within Collaborative Drug Discovery (Collaborative Drug Discovery USA) using a 4-parameter logistic fit. HTRF ratio calculation was performed using the following formula:

$$HTRF Ratio = \frac{{em}_{665 nm}}{{em}_{620 nm}} * 10 , 000$$

where em₆₆₅ₙₘ represents the measured emission at 665 nm upon excitation at 337 nm and em₆₂₀ₙₘ the measured emission at 620 nm upon excitation at 337 nm. The 4 parameter logistic fitting model was performed using the following formula:

$$= {Y}_{min} + \frac{( {Y}_{Max} - {Y}_{Min} )}{( 1 + {( \frac{{IC}_{50}}{X} )}^{HillSlope} )}$$

where Y represents the HTRF ratio response (as defined previously), X the compound concentration in μM, Yₘᵢₙ the minimum response plateau, Yₘₐₓ the maximum response plateau, IC₅₀ the concentration of agonist that gives a response half way between Yₘᵢₙ and Yₘₐₓ and HillSlope the steepness of the family of curves. No fitting model constraints was applied on Yₘᵢₙ, or Yₘₐₓ whilst IC₅₀>0 and −0.5>HillSlope>−3

### Cheng-Prusoff Transformation

Ki values were derived from the geometric mean of the IC₅₀ values using the Cheng-Prusoff transformation:

$${K}_{i} = \frac{{IC}_{50}}{1 + ( \frac{[ L ]}{{K}_{d}} )} = \frac{{IC}_{50}}{1 + ( \frac{0.1 uM}{0.1216 uM} )}$$

where [L] represents the concentration of fluorescent probe in μM, K_{d} the affinity (binding constant) of the fluorescent probe in units of μM and IC₅₀ the concentration of agonist that gives a response halfway between Yₘᵢₙ and Yₘₐₓ (as described in 1.2.1)

### Statistical Methods or Analyses

Analysis and IC₅₀ values were derived using KNIME analytics (KNIME, Zurich) transformation and fitting within Collaborative Drug Discovery (Collaborative Drug Discovery, USA) as described in 1.2.1. Ki values were derived from the geometric mean of the IC₅₀ values using the Cheng-Prusoff transformation as described in 2.2.2. Data was visualized in GraphPad Prism 8.1.2 (GraphPad, USA) and reported as mean and standard deviation. Microsoft Office Excel 2012 (Redmond, WA) was used for calculation of mean and standard deviation.

IC₅₀ data are reported in Table 3 for compounds in Table 1. In Table 3 below, According to the code, A represents a IC₅₀ value of ≤0.1 μM, B represents a IC₅₀ value≥0.1 μM and ≤1 μM, C represents an IC₅₀ value>1 μM.

TABLE 3
HTRF CRBN & Ternary Complex data

| | HTRF_Thali_Disp_DR_{—} |
|---|---|
| Cpmd No. | geo_IC₅₀ (μM) |
| 101 | B |
| 102 | B |
| 103 | B |
| 104 | B |
| 105 | C |
| 106 | B |
| 107 | B |
| 108 | B |
| 109 | B |
| 110 | B |
| 111 | B |
| 112 | B |
| 114 | B |
| 115 | B |
| 116 | B |
| 117 | B |
| 118 | B |
| 119 | B |
| 120 | B |
| 121 | B |
| 122 | B |
| 123 | B |
| 124 | A |
| 125 | B |
| 126 | B |
| 127 | B |
| 128 | B |
| 129 | B |
| 130 | B |
| 131 | B |
| 132 | B |
| 133 | B |
| 134 | B |
| 135 | B |
| 136 | B |
| 137 | B |
| 139 | B |
| 140 | B |
| 141 | B |
| 142 | B |
| 143 | B |
| 144 | B |
| 146 | B |
| 147 | B |
| 148 | B |
| 156 | B |
| 159 | B |
| 158 | B |
| 155 | A |
| 157 | A |
| 154 | B |

### Example 58. GI₅₀ Data

SKCO1, LS180, and LS174T cell lines were purchased from ATCC. CW2 cell line was purchased from Riken. GP2D cell line was purchased from Sigma/ECACC. All cell lines were cultured in manufacturer's recommended media at 37° C., 5% CO₂. Briefly, cells were suspended in 200 μL of media and seeded at 300 to 1,800 cells per well in tissue culture treated 96-well plates with black walls and clear bottom. Plates were incubated overnight and initial (T0) read was performed the following day using CyQUANT Direct Cell Proliferation Assay Kit (Thermo Fisher Scientific) according to manufacturer's protocol. All compounds were solubilized in DMSO and prepared at 3-fold serial dilution, using 9 points starting at 30 μM as highest concentration, administered to cells and incubated for 120 to 168 hours, at which point CyQUANT assay measurement was performed using Acumen Cellista instrument (Ex/Em 480/520 nm). Media-only wells were used as blank, and 0.3% DMSO was used as DMSO-treated cell control. Growth inhibition (percent response) was calculated using the following formula in Excel: “=if ([Compound treated Day N]>[Untreated Day 0, AVG], ([Compound treated Day N]−[Untreated Day 0, AVG])/([Untreated Day N]−[Untreated Day 0, AVG])*100, ([Compound Treated Day N]−[Untreated Day 0, AVG])/([Untreated Day 0, AVG])*100)”. GI₅₀ is the response corresponding to the 50% of untreated control.

GI₅₀ data are reported in Tables 4a, 4b, 4c, 4d, and 4e for compounds in Table 1. In Tables 4a, 4b, 4c, 4d, and 4e below, According to the code, A represents a GI₅₀ value of ≤0.1 μM, B represents a GI₅₀ value>0.1 μM and ≤1 μM, C represents an GI₅₀ value>1 μM.

TABLE 4a
SKCO1 GI₅₀ data

| | SKCO1 |
|---|---|
| Cpmd No. | GI₅₀ (μM) |
| 154 | A |
| 109 | A |
| 147 | B |

TABLE 4b
CW2 GI₅₀ data

| | CW2 |
|---|---|
| Cpmd No. | GI₅₀ (μM) |
| 154 | A |
| 109 | A |
| 147 | B |

TABLE 4c
GP2D GI₅₀ data

| | GP2D |
|---|---|
| Cpmd No. | GI₅₀ (μM) |
| 154 | B |
| 109 | A |
| 147 | C |

TABLE 4d
LS180 GI₅₀ data

| | LS180 |
|---|---|
| Cpmd No. | GI₅₀ (μM) |
| 154 | B |
| 109 | B |
| 147 | C |

TABLE 4e
LS174T GI₅₀ data

| | LS174T |
|---|---|
| Cpmd No. | GI₅₀ (μM) |
| 154 | B |
| 109 | A |
| 147 | C |

### Example 59. Pharmacokinetics of Oral Absorption for Compound 154 and Compound 147

### Methods

Male BALB/c mice were fasted overnight and administered a discrete, PO dose of Compound 154 or Compound 147 at 10 mg/kg.

Blood samples were collected serially from three animals/time-point following PO administration of the test compounds to the mice and placed in tubes containing K₂EDTA. The blood samples were centrifuged, and the aliquots of the resulting plasma stored frozen at −60 to −90° C.

The plasma samples were analyzed by LC-MS/MS to determine concentrations of the test compounds. The in-life portion of the study and the bioanalysis of plasma samples was conducted at Wuxi (China).

### Data Analysis

The pharmacokinetic parameters for compound 154 or compound 147 were calculated by non-compartmental analysis using Phoenix WinNonlin software.

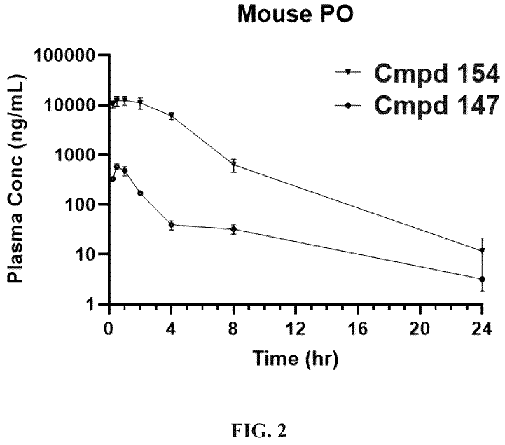

The pharmacokinetic parameters for mouse pharmacokinetic at 10 mg/kg for exemplary compounds are shown in Table 5. FIG. 2 shows the plasma concentration at different timepoints for compound 154 and compound 147.

TABLE 5
PK Parameters for mouse PK at 10
mg/kg for exemplary compounds

Mean ± SD

| | Compound 154 | Compound 147 |
|---|---|---|
| PK Parameters | 10 mg/kg | 10 mg/kg |
| T_{1/2} | 2.19 ± 0.24 | N/A |
| (hr) | | |
| Tmax | 1 | 0.5 |
| (hr) | | |
| Cₘₐₓ | 12869 ± 2561 | 574 ± 76.8 |
| (ng/mL) | | |
| AUCₗₐₛₜ | 50867 ± 10345 | 1167 ± 258 |
| (hr*ng/mL) | | |
| AUCₐₗₗ | 50905 ± 10365 | 1214 ± 235 |
| (hr*ng/mL) | | |

### EQUIVALENTS AND SCOPE

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula (I): 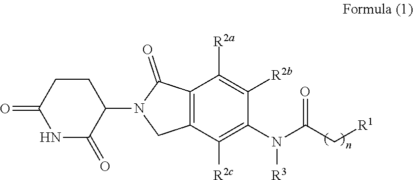
or a pharmaceutically acceptable salt thereof,
wherein:
R¹ is selected from the group consisting of phenyl, heteroaryl, cycloalkyl, and heterocyclyl, wherein each of phenyl, heteroaryl, cycloalkyl, and heterocyclyl is optionally substituted with one or more occurrences of R^{A};
each of R², R^{2b}, and R^{2c} is independently selected from the group consisting of H, C₁₋₃alkyl, and C₁₋₃alkoxy;
each occurrence of R^{A} is independently selected from the group consisting of oxo, halogen, C₁₋₆alkyl, C₁₋₃alkoxy, cyano, cycloalkyl, heteroaryl, and phenyl, wherein each of C₁₋₆alkyl, cycloalkyl, heteroaryl, and phenyl is optionally substituted with one or more occurrences of R^{B}, or one or more groups of two occurrences of R^{A}, together with the carbon to which they are attached, form oxo;
each occurrence of R^{B} is independently selected from the group consisting of halogen, C₁₋₆alkyl, and C₁₋₆alkoxy;
R³ is hydrogen, C₁₋₃alkyl, or C₁₋₃haloalkyl,
or R¹ and R³ can, together with the atoms to which they are attached, form a 9-10 membered heterocyclyl optionally substituted by one or more oxo; and
n is 0 or 1.

2. The compound of claim 1, wherein R¹ is 5 or 6-membered heteroaryl optionally substituted with one or more occurrences of R^{A}.

3. The compound of claim 1, wherein R¹ is phenyl optionally substituted with one or more occurrences of R^{A}.

4. The compound of claim 1 or 2, wherein R¹ is pyridinyl optionally substituted with one or more occurrences of R^{A}.

5. The compound of claim 1 or 2, wherein R¹ is thiazolyl, imidazolyl or oxazolyl optionally substituted with one or more occurrences of R^{A}.

6. The compound of claim 1 or 2, wherein R¹ is 5 or 6-membered heteroaryl substituted with one or more occurrences of R^{A}.

7. The compound of claim 1 or 3, wherein R¹ is phenyl substituted with one or more occurrences of R^{A}.

8. The compound of claim 1, 2, or 6, wherein R¹ is 5 or 6-membered heteroaryl substituted with one or two occurrences of R^{A}.

9. The compound of claim 1, 2, 6, or 8, wherein R¹ is pyridinyl substituted with one or two occurrences of R^{A}.

10. The compound of claim 1, 3, or 7, wherein R¹ is phenyl substituted with one or two occurrences of R^{A}.

11. The compound of claim 1, 2, 6, or 8, wherein R¹ is 5 or 6-membered heteroaryl substituted with one or two occurrences of halogen.

12. The compound of claim 1, 2, 6, 8, or 9, wherein R¹ is pyridinyl substituted with one or two occurrences of halogen.

13. The compound of claim 1, 3, 7, or 10, wherein R¹ is phenyl substituted with one or two occurrences of halogen.

14. The compound of claim 1, 2, 6, or 8, wherein R¹ is 5 or 6-membered heteroaryl substituted with methyl, wherein methyl is optionally substituted with one more occurrences of halogen.

15. The compound of claim 1, 2, 6, 8, or 14 wherein R¹ is pyridinyl substituted with methyl, wherein methyl is optionally substituted with one more occurrences of halogen.

16. The compound of claim 1, 3, 7, or 10, wherein R¹ is phenyl substituted with methyl, wherein methyl is optionally substituted with one more occurrences of halogen.

17. The compound of claim 1, wherein R¹ is 8-10 membered bicyclic heterocyclyl optionally substituted by one or more oxo.

18. The compound of claim 17, wherein R¹ is indolinyl optionally substituted by one or more oxo.

19. The compound of claim 1, wherein R¹ is 9-10 membered bicyclic cycloalkyl.

20. The compound of claim 1, wherein R¹ is 5-6 membered monocyclic heterocyclyl optionally substituted by one or more C₁₋₆alkyl, wherein the alkyl is optionally substituted by one or more halogen.

21. The compound of any one of claims 1-20, wherein R^{2a}, R^{2b}, and R^{2c} are H.

22. The compound of any one of claims 1-20, wherein each of R^{2a}, R^{2b}, and R^{2c} is independently selected from the group consisting of C₁₋₃alkyl and C₁₋₃alkoxy.

23. The compound of claim 22, wherein each of R^{2a}, R^{2b}, and R^{2c} is independently selected from the group consisting of methyl, ethyl, and methoxy.

24. The compound of any one of claims 1-23, wherein each occurrence of R^{A} is independently selected from the group consisting of halogen, C₁₋₃alkyl, and cyano, wherein C₁₋₃alkyl is optionally substituted with one or more occurrences of halogen.

25. The compound of any one of claims 1-24, wherein each occurrence of R^{A} is independently selected from the group consisting of F, Cl, methyl, ethyl, isopropyl, and cyano, wherein each of methyl, ethyl, and isopropyl is optionally substituted with one or more occurrences of F.

26. The compound of any one of claims 1-25, wherein each occurrence of R^{A} is independently selected from the group consisting of C₁ and methyl.

27. The compound of any one of claims 1-23, wherein each occurrence of R^{A} is independently selected from the group consisting of phenyl and heteroaryl.

28. The compound of any one of claims 1-27, wherein each occurrence of R^{B} is independently selected from the group consisting of F, Cl, methyl, ethyl, and cyano, wherein each of methyl and ethyl is optionally substituted with one or more occurrences of F.

29. The compound of any one of claims 1-28, wherein R^{t} is selected from the group consisting of:

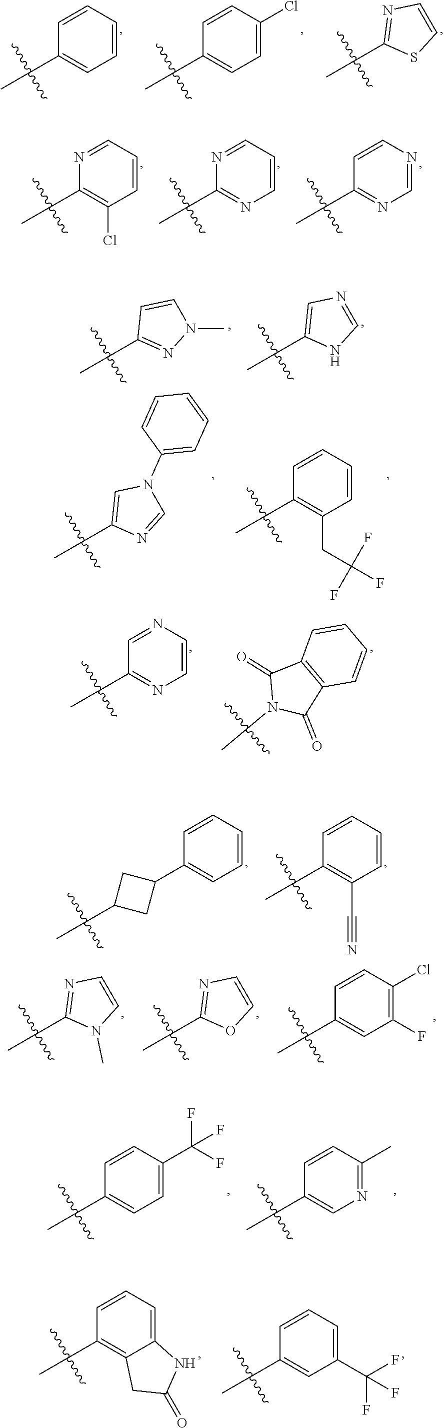

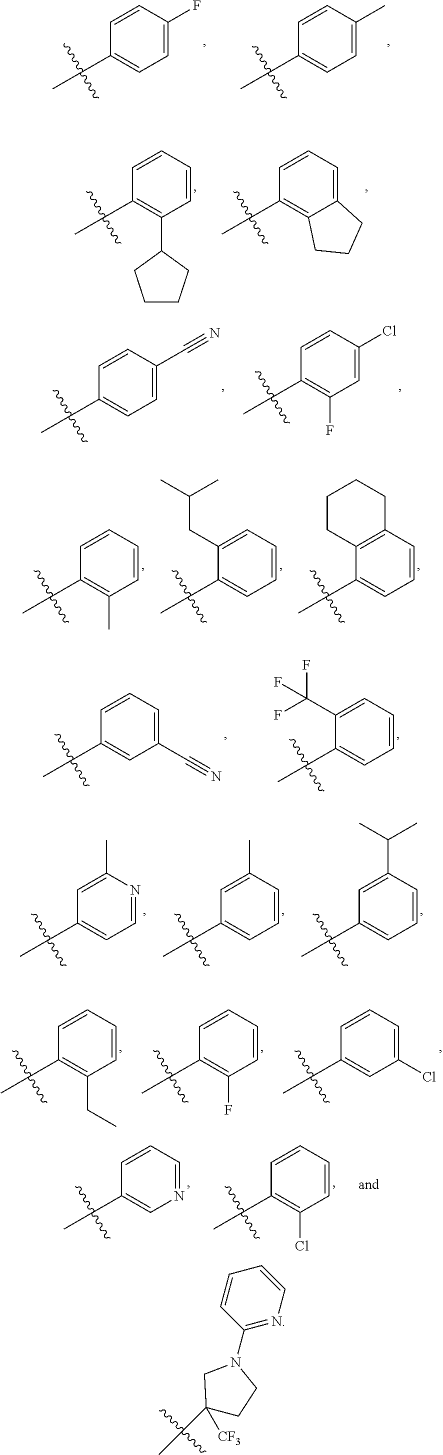

30. The compound of claim 29, wherein R¹ is selected from the group consisting of:

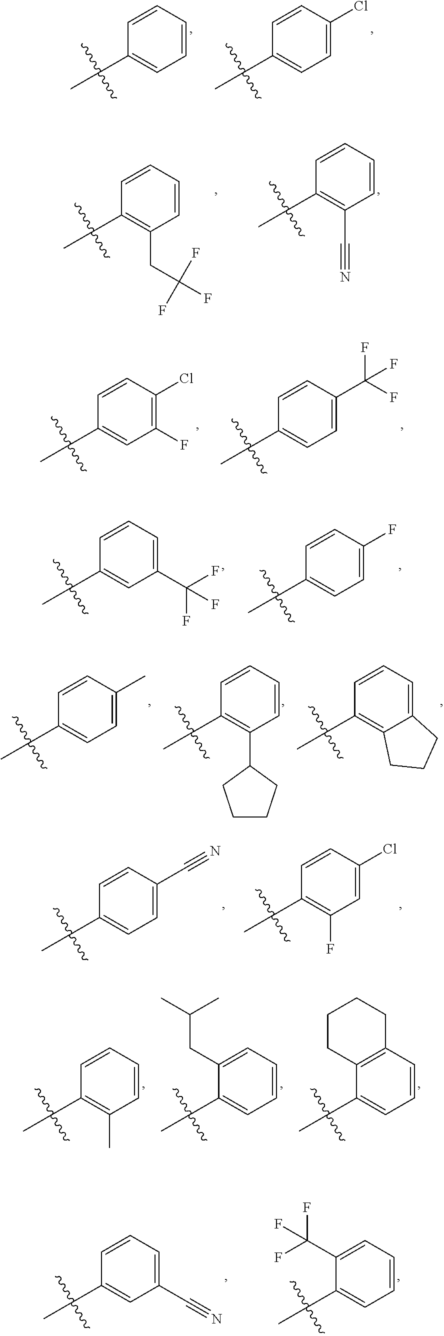

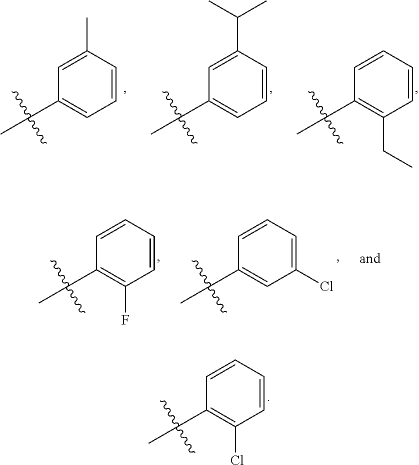

31. The compound of claim 27, wherein R¹ is selected from the group consisting of:

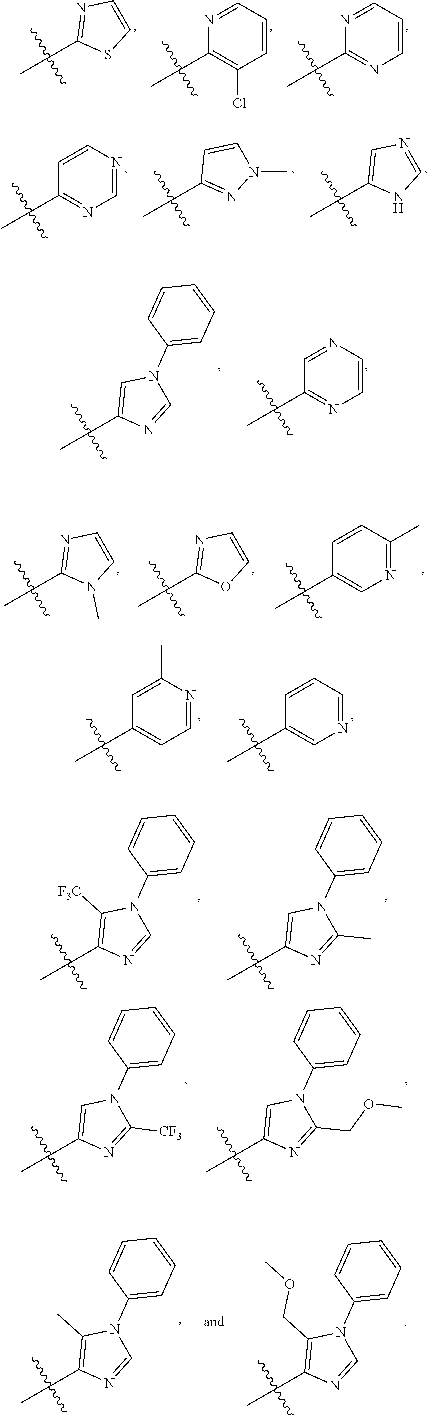

32. The compound of any one of claims 1-31, n is 1.

33. The compound of any one of claims 1-31, n is 0.

34. A compound selected from the group consisting of: 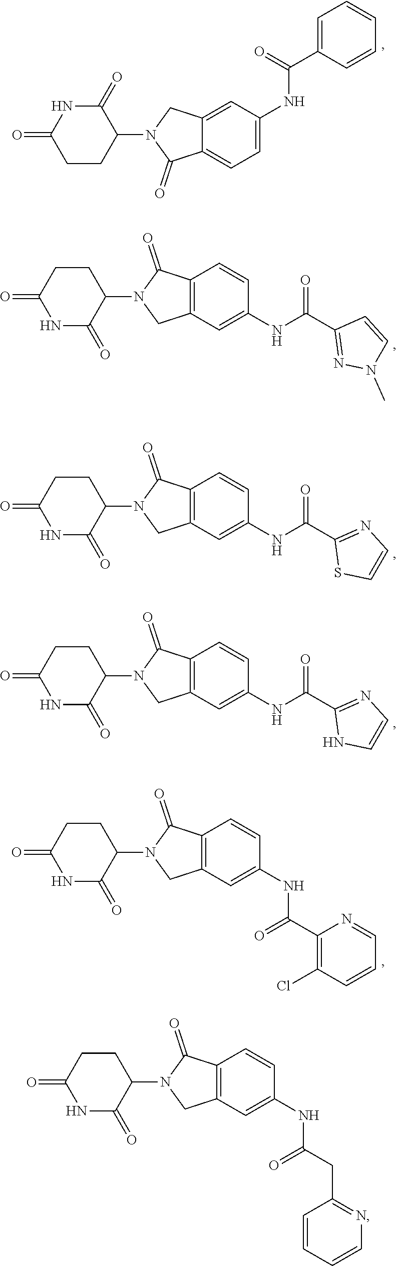 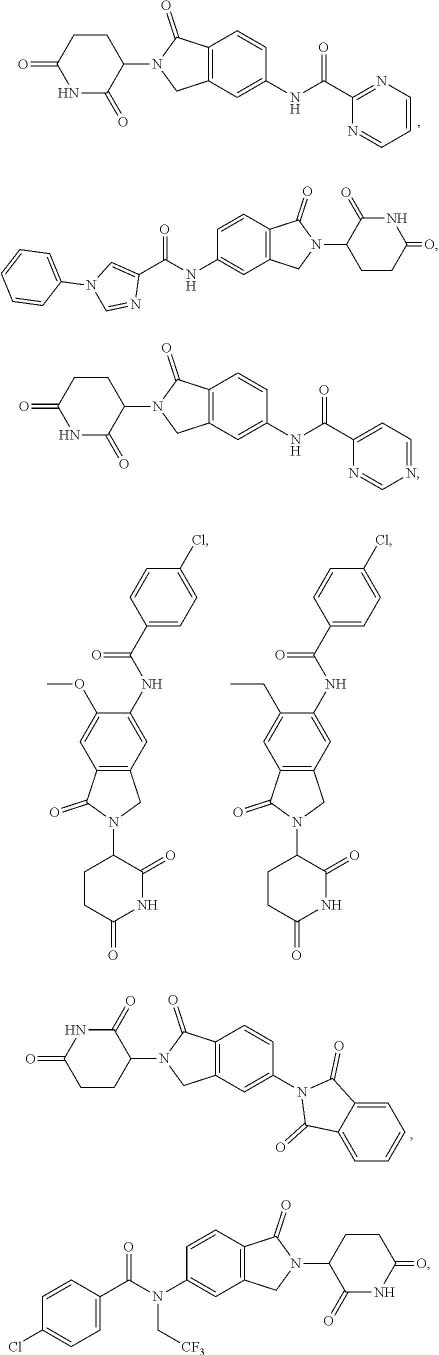 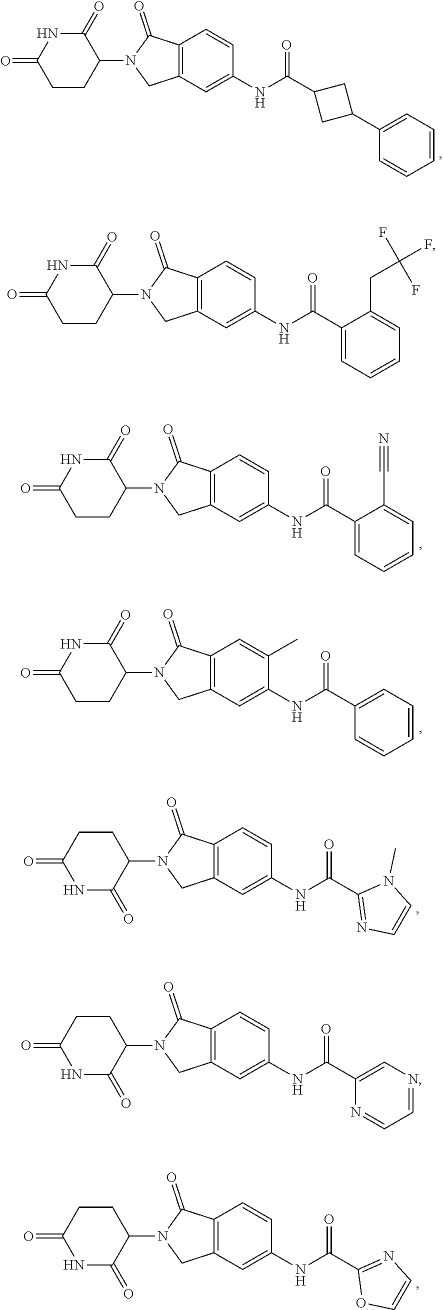 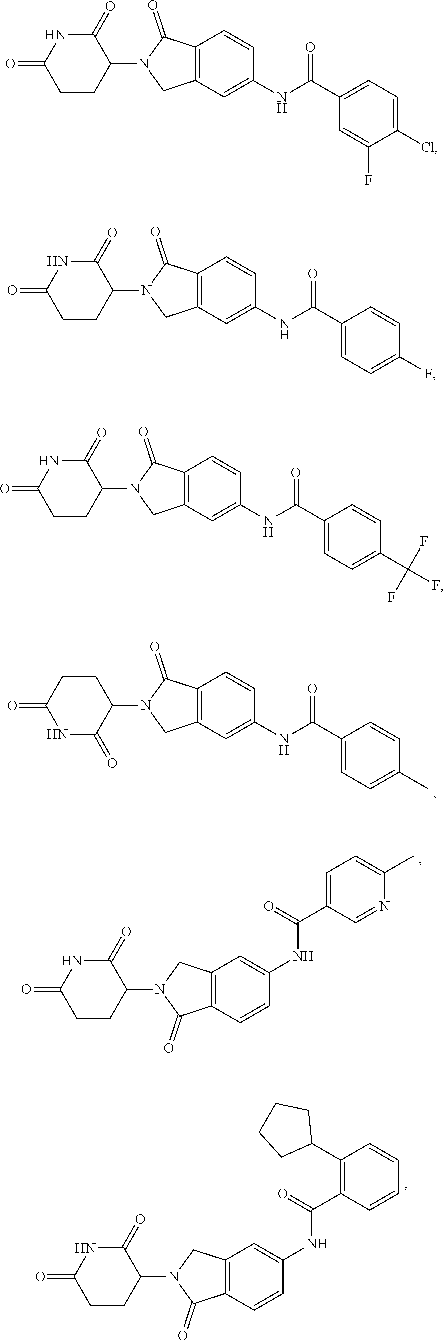 [SMILES: O=C1CCC(N2Cc3cc(NC(=O)c4ccc(Cl)c(F)c4)ccc3C2=O)C(=O)N1 ; O=C1CCC(N2Cc3cc(NC(=O)c4ccc(F)cc4)ccc3C2=O)C(=O)N1 ; O=C1CCC(N2Cc3cc(NC(=O)c4ccc(C(F)(F)F)cc4)ccc3C2=O)C(=O)N1 ; Cc1ccc(C(=O)Nc2ccc3c(c2)CN(C2CCC(=O)NC2=O)C3=O)cc1 ; Cc1ccc(C(=O)Nc2ccc3c(c2)CN(C2CCC(=O)NC2=O)C3=O)cn1 ; O=C1CCC(N2Cc3cc(NC(=O)c4ccccc4C4CCCC4)ccc3C2=O)C(=O)N1] 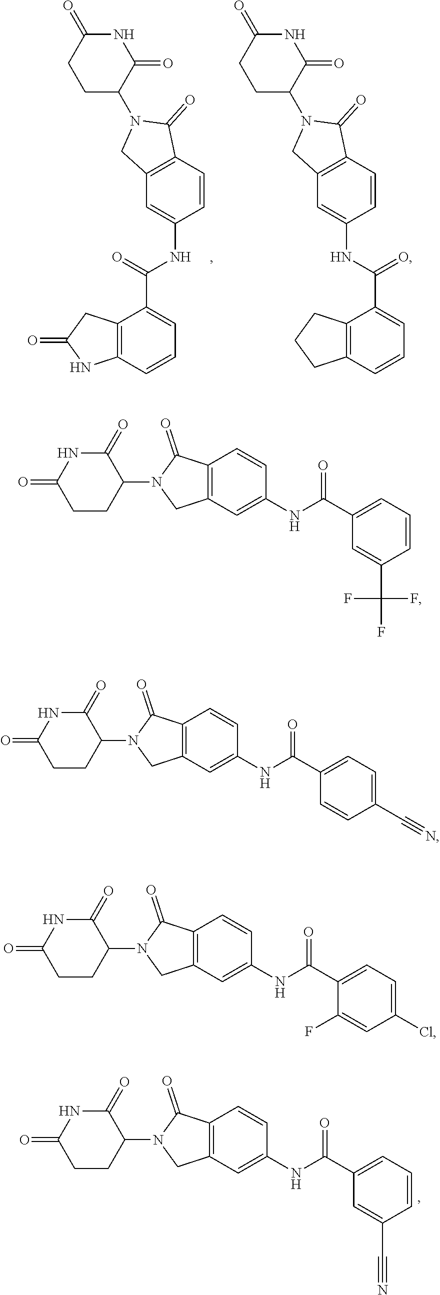 [SMILES: O=C1CCC(N2Cc3cc(NC(=O)c4cccc5c4CC(=O)N5)ccc3C2=O)C(=O)N1 ; O=C1CCC(N2Cc3cc(NC(=O)c4cccc5c4CCC5)ccc3C2=O)C(=O)N1 ; O=C1CCC(N2Cc3cc(NC(=O)c4cccc(C(F)(F)F)c4)ccc3C2=O)C(=O)N1 ; N#Cc1ccc(C(=O)Nc2ccc3c(c2)CN(C2CCC(=O)NC2=O)C3=O)cc1 ; O=C1CCC(N2Cc3cc(NC(=O)c4ccc(Cl)cc4F)ccc3C2=O)C(=O)N1 ; N#Cc1cccc(C(=O)Nc2ccc3c(c2)CN(C2CCC(=O)NC2=O)C3=O)c1] 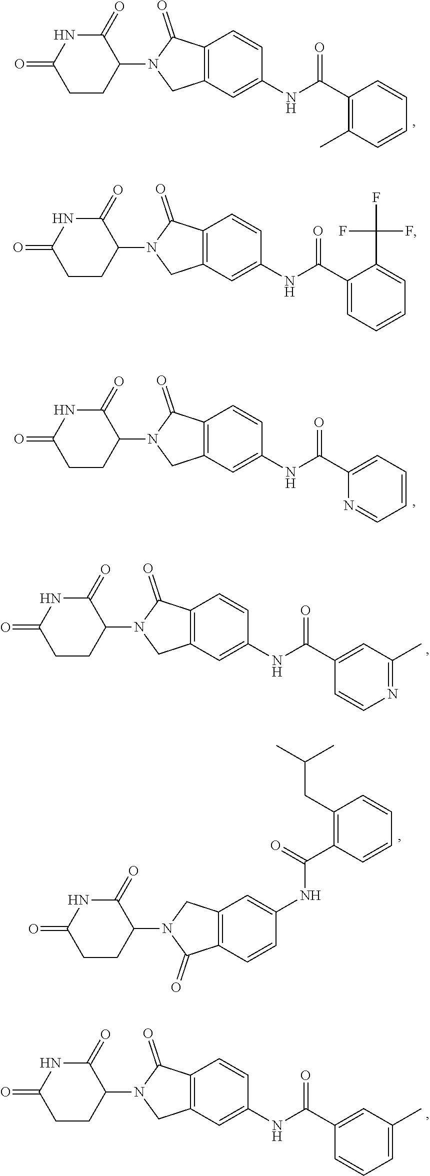 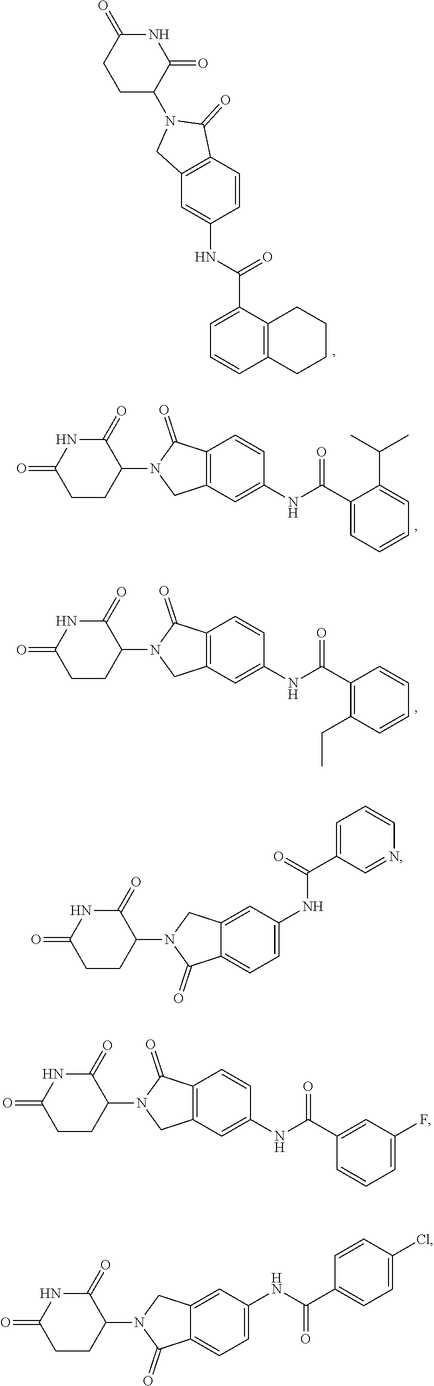 [SMILES: O=C1CCC(N2Cc3cc(NC(=O)c4cccc5c4CCCC5)ccc3C2=O)C(=O)N1 ; CC(C)c1ccccc1C(=O)Nc1ccc2c(c1)CN(C1CCC(=O)NC1=O)C2=O ; CCc1ccccc1C(=O)Nc1ccc2c(c1)CN(C1CCC(=O)NC1=O)C2=O ; O=C1CCC(N2Cc3cc(NC(=O)c4cccnc4)ccc3C2=O)C(=O)N1 ; O=C1CCC(N2Cc3cc(NC(=O)c4cccc(F)c4)ccc3C2=O)C(=O)N1 ; O=C1CCC(N2Cc3cc(NC(=O)c4ccc(Cl)cc4)ccc3C2=O)C(=O)N1] 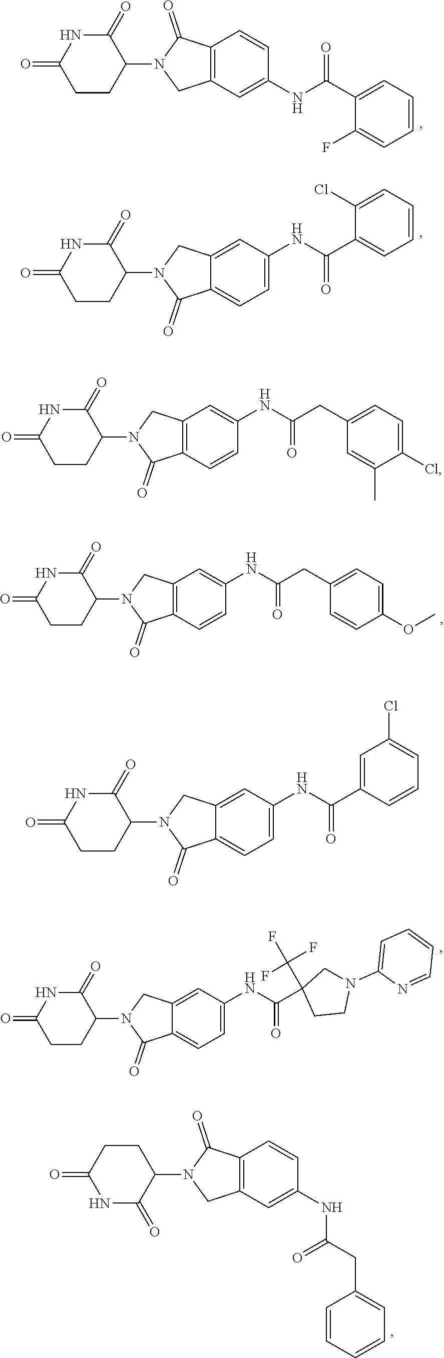 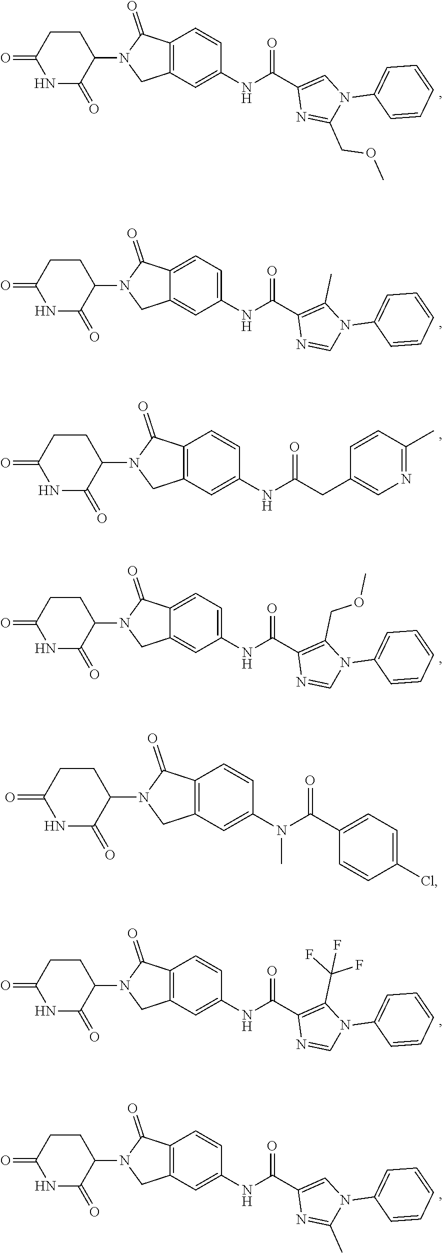 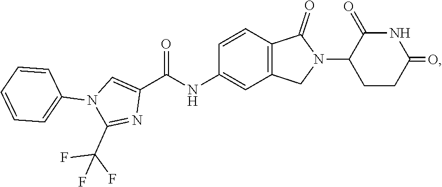 [SMILES: O=C1CCC(N2Cc3cc(NC(=O)c4cn(-c5ccccc5)c(C(F)(F)F)n4)ccc3C2=O)C(=O)N1]
and pharmaceutically acceptable salts thereof.

35. A pharmaceutical composition comprising the compound of any one of claims 1-34, or pharmaceutically acceptable salt thereof, and a pharmaceutically acceptable excipient.

36. A method of degrading CK1a in a subject suffering from cancer, comprising administering to the subject an effective amount of the compound of any one of claims 1-34, or pharmaceutically acceptable salt thereof, or the pharmaceutical composition of claim 35.

37. A method of treating cancer in a subject in need thereof, comprising administering to the subject a therapeutically effective amount of the compound of any one of claims 1-34, or pharmaceutically acceptable salt thereof, or the pharmaceutical composition of claim 35.

38. A method of treating a solid tumor in a subject in need thereof, comprising administering to the subject a therapeutically effective amount of the compound of any one of claims 1-34, or pharmaceutically acceptable salt thereof, or the pharmaceutical composition of claim 35.

39. A method of treating a liquid tumor in a subject in need thereof, comprising administering to the subject a therapeutically effective amount of the compound of any one of claims 1-34, or pharmaceutically acceptable salt thereof, or the pharmaceutical composition of claim 35.

40. The method of any one of claims 36-39, further comprising administering to the subject an additional therapeutic agent.
